(19) United States
(12) Patent Application Publication
(10) Pub. No.: US 2025/0253030 A1
(43) Pub. Date: Aug. 7, 2025

(54) AMELIORATING EXPERIENTIAL NEGATIVE SYMPTOMS OF SCHIZOPHRENIA IN SUBJECTS USING DIGITAL THERAPEUTICS WITH ADAPTIVE GOAL SETTING

(71) Applicant: Click Therapeutics, Inc., New York, NY (US)

(72) Inventors: Eehwa Ung, San Francisco, CA (US); Tim Campellone, Oakland, CA (US); Demetrius Johnson, San Francisco, CA (US); Puneet Sodhi, Columbus, OH (US)

(73) Assignee: Click Therapeutics, Inc., New York, NY (US)

(21) Appl. No.: 18/432,618

(22) Filed: Feb. 5, 2024

Related U.S. Application Data
(60) Provisional application No. 63/483,512, filed on Feb. 6, 2023.

(51) Int. Cl. G16H 20/70; A61B 5/00; A61B 5/16; A61K 31/422; A61K 31/496; A61K 31/519; A61K 31/5513; A61K 31/554; G16H 20/10

(52) U.S. Cl. CPC G16H 20/70; A61B 5/165; A61B 5/4848; A61K 31/422; A61K 31/496; A61K 31/519; A61K 31/5513; A61K 31/554; G16H 20/10

(57) ABSTRACT

Provided herein are methods of ameliorating experiential negative symptoms of schizophrenia in a user in need thereof. A computing system may obtain a first metric associated with the user prior to a plurality of time instances. The computing system may repeat across a plurality of time instances, identifying a configuration file selected based on an endpoint for ameliorating the experiential negative symptoms. The computing system may repeat presenting the set of content items identified by the configuration file to prompt the user to perform the activity towards achieving the endpoint. The computing system may obtain a second metric associated with the user after the plurality of time instances. The user may show amelioration of experiential negative symptoms of schizophrenia, when the second metric is lower than the first metric. The computing system may increase the efficacy of the medication that the user is taking to address the schizophrenia.

### CROSS REFERENCES TO RELATED APPLICATIONS

The present application claims the benefit of priority under 35 U.S.C. § 119 (e) to U.S. Patent Provisional Application No. 63/483,512, titled “Combination Therapy with Digital Therapeutics and Antipsychotics in Subjects with Experiential Negative Symptoms of Schizophrenia,” filed Feb. 6, 2023, which is incorporated herein in its entirety by reference.

### BACKGROUND

Schizophrenia is the leading cause of disability after adjusting for prevalence rates. Given the chronic nature of schizophrenia, people with schizophrenia often have poor education, reduced quality of life, difficulty living independently, and socio-occupational dysfunction. This impairment is so severe that only 10% to 20% of people with schizophrenia work fulltime or part-time, and most people with schizophrenia require public funding, with lost productivity as a major driver of cost. In addition to the patient burden, there is a significant burden for the caregivers of people with schizophrenia. Such disability, and the resulting patient and caregiver burden, typically persists or worsens over time, which is confirmed by a recent meta-analysis finding that only 14% of people with schizophrenia achieve functional recovery.

In addition to disability, schizophrenia exacts a heavy personal burden. Mortality rates are 2.5 times higher than those found in the general population, and people with schizophrenia die 28.5 years earlier than the general population. This staggering degree of early mortality is largely driven by high rates of suicide. People with schizophrenia have a 20 times higher rate of suicidality than the general population. This results in people with schizophrenia comprising up to 23% of the people who die by suicide each year.

A significant contributor to the profound burden described above is the presence of significant negative symptoms. Negative symptoms are commonly reported as the first symptoms of schizophrenia. As such, negative symptoms present in 73% of people prior to the onset of positive symptoms, in 90% of those experiencing a first episode of psychosis, and they persist in 35 to 70% of people with schizophrenia even after treatment initiation. Despite their early onset, negative symptoms are not effectively addressed by standard of care (SOC) or any other medication, and are strong predictors of functional impairment, as they lead to increased functional worsening over time.

Current guidelines for schizophrenia recommend antipsychotic medications and adjunctive psychosocial intervention. Antipsychotic medications are efficacious in treating positive symptoms, but are associated with burdensome side effects, which results in high rates of medication noncompliance. In addition, no pharmacological interventions are available for the treatment of negative symptoms. Therefore, adjunctive psychosocial intervention is recommended for symptoms not well treated by pharmacological intervention, such as negative symptoms.

Significant barriers to treatment access and adherence limit the efficacy of standard of care (SOC) in addressing negative symptoms. First and foremost, there is currently no drug, biological product, or medical device approved or cleared as an adjunctive treatment for experiential negative symptoms in schizophrenia. Secondly, while adjunctive psychosocial intervention is consequently recommended to address negative symptoms, there is a shortage of psychiatrists as well as other adequately trained clinicians to provide treatment, which is further exacerbated by an implementation gap in training community providers to follow evidence-based practices. These limitations result in as few as 10% of patients with schizophrenia receiving evidence-based adjunctive psychosocial interventions for their negative symptoms. Thus, an unmet need exists for accessible treatments that target and improve negative symptoms.

An application may be deployed and installed on a computing device to provide a digital therapeutic to address a behavioral or psychological condition of a user. As part of the digital therapeutic, the application itself may be configured to present a user interface containing various elements for display and carry out a multitude of operations in response to user interactions with elements of the user interface. After deployment, it may be difficult for the application to dynamically adapt to the responses from the user, leading to a lower likelihood that the user is to interact with the application. As a result, the efficacy of the digital therapeutic and user adherence to the digital therapeutic may be greatly diminished.

In a network environment, an application on a client may present one or more content items received as part of a message from a server. The content items may be customized for the particular user of the client and may be selected based on any number of factors, such as user history, device type, location, or time, among others. The content items may also contain a script (e.g., event listeners or handlers) to specify presentation functionality when loaded onto the application of the client. For example, the content items may be provided as a part of a digital therapeutics platform and may include a textual content related to a condition associated with the user (e.g., smoke cessation, dieting, exercising, and psychological illnesses). Upon receipt, the application on the client may display the content items as user interface elements in a graphical user interface (GUI) of the application. As the content items are displayed via the application, the user may interact with one or more of the user interface elements to trigger the functionality of the corresponding content items.

While the provision of content items in this manner may carry out the specified functionalities, the range of possible functionalities and the selection of additional, subsequent content items for the user may be highly limited. In the context of digital therapeutics, the selection and provision of content items may be inadequate at achieving an endpoint associated the condition with the user. For one, the digital therapeutics application itself may have relatively static and fixed functionality once deployed and installed on the client, without resorting to updating the application. For instance, the application once installed may provide limited number of different configurations for graphical user interfaces in which to present the content items. For another, the state and behavior of the user may change throughout time, and the information of the factors used to select content items in advance may be inadequate to adapt to the related ever-changing state of the user.

In addition, the content items themselves may lack logic at accounting for such changes, and thus may be too generic and irrelevant to the state of the user at the time of presentation, leading to poor quality of human-computer interaction (HCl) between the application and the user. These may result in the expenditure of computing resources from loading and rendering these content items on the application, and consumption of network bandwidth from back-and-forth communications between the client and the server. From an HCl perspective, the lack of adaptability of the content items may lead to lower user engagement and lower adherence to the digital therapeutics provided through the presentation of the content items.

### SUMMARY

To address these and other challenges, presented herein are systems and methods for dynamically selecting configuration files for applications in accordance with an adaptive goal setting (AGS) framework. The configuration files can be selected and personalized for the user initially based on baseline measurements of the user and then subsequently followed by response data from the user. The provision of digital therapeutic via the configuration files on the application in this manner may increase user engagement and adherence and ultimately the efficacy of the digital therapeutic. The AGS framework can continuously and dynamically configure the application to be tailored toward the user and their endpoints, by capturing the user's interest (meeting users where they are) and providing personalized content, and leveling goals based on baseline proficiency and metrics (matching goals with where the user is in their current functioning). The framework may provide for daily activity engagement (e.g., multiple times a day) to the user to take advantage of the application on their personal mobile devices to increase engagement and likelihood of therapeutic efficacy (compared to the traditional model, which only allows for interaction between a physician and a user when the user can visit a clinic in-person, which might be once a week, if that). The AGS framework may also provide for mood mitigation strategies to address user's states and to increase retention. In addition, the AGS framework may include personalized features such as a daily mood check-in, pre- and post-activity rating self-assessment, personal values exploration, and a “do it now” option.

The server (or a computing device) may select configuration files to provide to the application for presentation of content. Each configuration file may include logic or a module for an automaton (e.g., a finite state machine (FSM)) for one or more endpoints specifying one or more activities to be performed via the application on the client within a set time period. For digital therapeutics applications, the configuration file may identify a set of states and a set of transitions. Each state may correspond to a level associated with the activities of the endpoint and may specify activities of the level to be performed via the application. Each transition may correspond to a condition to satisfy to move from one state to the next. The condition may be, for example, success or failure at carrying out the specified activities within the set time period.

The selection, provision, and the internal logic of the configuration files themselves may be in accordance with a framework for adaptive goal setting (AGS). The AGS framework may support the performance of activities for an endpoint (also referred herein as a goal, target, or objective) to address a condition of the user in an adaptive manner. The framework may include an introduction of a skill, setting up of a plan to achieve the endpoint, keeping track of the progress of the user, and adaptively varying the activities to incorporate for the endpoint.

To that end, during the introduction phase, a user profile may be constructed using user responses to prompts (e.g., questionnaire) related to the condition and endpoint for the user. The user profile may then be used to determine one or more endpoints and select configuration files to effectuate the endpoints as part of a plan to address the condition of the user. This AGS framework may be used to modulate, for example, different categories of objectives (e.g., smoke cessation, dieting to lower weight, improving exercise routine, and addressing symptoms of psychological illnesses); different types of activities (e.g., walking, running, swimming, hiking, dancing, and stair climbing); different difficulty levels of activities (e.g. lead climbing, top rope climbing, bouldering); a duration of activities (e.g., 5 minutes, 10 minutes, and 1 hour), a frequency of the activities (e.g., 1 time a week, 1 time a day, and 3 times a day), and a time of day in which to perform the activity (e.g., morning, afternoon, and after meals), among others.

The configuration file may provide the automaton logic with a persistent state, so that the user progress can be maintained across usage sessions. The configuration file itself may be a plug-in for the application and may be modified (e.g., by a clinician) for a particular endpoint, without relying on reconfiguration of the application. The automaton logic may be used to hold a set of variables, such as a level, an activity, an endpoint, and frequency, among others. For example, one set of variables may correspond to a level four of Activity A for 5 minutes every other day in the morning for a user. Given this information, the configuration file may be constructed to provide a set of content items to guide the user to perform and record the activity via the application on the client. As the user progresses through the automaton logic, the level, activity, endpoint, and frequency may be updated to provide a customized experience at each state. The logic may modulate the content items up or down to meet the current state of the user based on the responses via the application. In addition, over the course of the time period, different endpoints may be adaptively determined using response data and various configuration files may be dynamically selected in accordance with the endpoint to provide to the application.

By providing the configuration files in this manner, the application may be customized upon request and may provide a greater range of user experiences to the user to meet different endpoints, thereby improving the HCl between the user and the application. As the content items can be selected based on the states associated with the user in accordance with the configuration file, the content items may lead to higher user engagement with the digital therapeutic application. With higher likelihood of engagement, the digital therapeutic provided by the application may have higher efficacy in addressing the condition and increased adherence of the user with the digital therapeutic. The configuration files may also reduce resorting to having to update the application itself to provide additional functionality. Dynamically selecting and providing the configuration files may decrease the consumption of computing resources (e.g., processor, memory, and network bandwidth) from providing and loading irrelevant content items on the application. Furthermore, the configuration files may reduce the expenditure of network bandwidth from back-and-forth communications associated with requesting and retrieving the content items.

Aspects of the present disclosure are directed to systems, methods, and non-transitory computer-readable media for selecting configuration files for applications. A computing system may maintain a plurality of configuration files readable by an application. Each of the plurality of configuration files may identify a respective set of content items to prompt users to perform at least one of a plurality of activities via the application towards achieving a respective endpoint of a plurality of endpoints. The computing system may determine an endpoint of the plurality of endpoints to address a condition of a user. The computing system may select, from the plurality of configuration files, a configuration file identifying a set of content items for an activity of the plurality of activities to be performed by the user via the application towards achieving the endpoint. The computing system may provide the configuration file to the application to present the set of content items to prompt the user to perform the activity via the application.

In some embodiments, the computing system may receive, from the application, response data identifying one or more interactions by the user with the set of content items presented via the application towards achieving the endpoint. The computing system may determine a second endpoint of the plurality of endpoints based on the response data for the endpoint. The computing system may select, from the plurality of configuration files, a second configuration file based on the second endpoint to provide to the application.

In some embodiments, the computing system may identify, from a profile of the user, a first level of a plurality of levels towards achieving the endpoint. The computing system may determine a transition from the first level to a second level, based on response data identifying one or more interactions by the user with the set of content items presented via the application. The computing system may select, from the plurality of configuration files, a second configuration file based on the transition to the second level. In some embodiments, the computing system may determine, based on a performance of the activity of the user towards achieving the endpoint, whether to transition to the same first level or a second level of the plurality of levels towards achieving a second endpoint.

In some embodiments, each of the plurality of configuration files may identify a respective criterion defining a first measure of the user to select a corresponding configuration file identifying the set of content items to prompt users to perform the activity. The computing system may determine a second measure based on a profile of the user. The second measure may identify at least one of (i) a likelihood that the user is to perform the activity or (ii) a predicted efficacy of the activity on the user towards achieving the endpoint. In some embodiments, the computing system may identify, from the plurality of activities, the activity based on a comparison of the first measure with the second measure.

In some embodiments, the computing system may receive, from the application, a response identifying a mood of the user in response to presentation of a prompt via the application at a defined time. In some embodiments, the computing system may identify, from the plurality of activities, the activity based on the mood of the user indicated in the response. In some embodiments, the computing system may receive, from the application, a response identifying a plurality of personal values of the user. In some embodiments, the computing system may identify, from the plurality of activities, the activity based on the plurality of personal values associated with the user identified in the response. In some embodiments, the computing system may determine a progression metric based on performance of the activity towards achieving the endpoint. In some embodiments, the computing system may present, via the application, a connection between the progression metric and the personal value associated with the user.

In some embodiments, the computing system may present, via the application, a prompt for the user to indicate a first rating associated with the activity prior to performance of the activity via the application. In some embodiments, the computing system may store a response identifying the first rating associated with the activity from the application. In some embodiments, the computing system may present, via the application, a prompt for the user to indicate a second rating associated with the activity subsequent to performance of the activity via the application. In some embodiments, the computing system may present, via the application, a comparison of the second rating with the first rating. In some embodiments, the computing system may determine, from the plurality of endpoints, the endpoint based on at least one of (i) a baseline assessment and (ii) an indication by the user requesting an activity towards achieving the endpoint.

In some embodiments, at least one configuration file of the plurality of configuration files may define a finite state machine. The finite state machine may identify a plurality of states including at least a first state and a second state, each of which corresponds to an intensity level for a corresponding activity and specifies an output. The output may identify the one or more content items to present via a user interface of the application. The finite state machine may identify a plurality of transitions, each of which specifies an event to be detected via the user interface of the application to move from the first state to the second state, the event corresponding to an interaction to be performed via the application for the respective activity.

In some embodiments, the plurality of endpoints may be associated with at least one of a plurality of classifications for endpoints. In some embodiments, a first subset of the plurality of endpoints may be associated with a first classification and a second subset of the plurality of endpoints may be associated with a second classification. In some embodiments, the user may be on a medication to address the condition, at least partially in concurrence with performance of the activity via the application. In some embodiments, the condition may include a psychological illness.

Aspects of the present disclosure are directed to methods of ameliorating experiential negative symptoms of schizophrenia in a user in need thereof. One or more processors may obtain a first metric associated with the user prior to a plurality of time instances. The one or more processors may repeat, for each of a plurality of time instances, identifying, for provision to an application, a configuration file selected from a plurality of configuration files based on an endpoint for ameliorating the experiential negative symptoms. The configuration file may identify a set of content items for an activity of a plurality of activities towards achieving the endpoint. The one or more processors may repeat presenting, via the application responsive to providing the configuration file to the application, a set of content items identified by the configuration file to prompt the user to perform the activity towards achieving the endpoint. The one or more processors may repeat receiving response data identifying one or more interactions by the user with the set of content items. The one or more processors may obtain a second metric associated with the user after at least one of the plurality of time instances. The user may show amelioration of experiential negative symptoms of schizophrenia, when the second metric is statistically different from the first metric.

In some embodiments, the first metric and second metric may include scores on at least one of a Clinical Assessment Interview for Negative Symptoms (CAINS) Motivation and Pleasure Scale, Clinical Assessment Interview for Negative Symptoms, Expressivity Scale (CAINS-EXP), Positive and Negative Syndrome Scale (PANSS), Personal and Social Performance Scale (PSP), a Defeatist Beliefs Subscale of the Dysfunctional Attitudes Scale (DAS), Patient Global Impression of Improvement Scale (PGI-I), Patient Global Impression of Severity Scale (PGI-S), Clinical Global Impression of Severity Scale (CGI-S), WHO Disability Assessment Schedule 2.0 (WHODAS 2.0), or Schizophrenia Quality of Life Scale-Revision 4 (SQLS-R4).

In some embodiments, the experiential negative symptoms of schizophrenia may include one or more of blunted affect, alogia (reduction in quantity of words spoken), avolition (reduced goal-directed activity due to decreased motivation), asociality, and anhedonia (reduced experience of pleasure). In some embodiments, the user may be an adult or a late adolescent. In some embodiments, the user has experienced at least moderate to severe negative symptom severity prior to the first activity. In some embodiments, the user may have a score of ≤30 on the Motivation and Pleasure Scale (MAPS) prior to the first activity.

In some embodiments, the user may be receiving a stable dose of an antipsychotic medication for at least 12 weeks prior to the first activity. In some embodiments, the medication may include risperidone, quetiapine, olanzapine, ziprasidone, paliperidone, aripiprazole, or iclepertin. In some embodiments, the user may be male, female, or non-binary.

In some embodiments, at least one of the plurality of configuration files may identify a criterion defining a measure of the user to select another of the plurality of configuration files, the measure identifying at least one of (i) a likelihood that the user is to perform the activity or (ii) a predicted efficacy of the activity on the user towards achieving the endpoint. In some embodiments, the endpoint for a first-time instance of the plurality of time instances may be selected from a plurality of endpoints based on a baseline assessment of the user for the experiential negative symptoms of schizophrenia.

In some embodiments, the endpoint may be selected from a plurality of endpoints based on a personal value indicated by the user. In some embodiments, the endpoint may be selected from a plurality of endpoints based on the response data identifying the one or more interactions by the user with the set of content items presented via the application from a previous time instance. In some embodiments, the endpoint may be selected from a plurality of endpoints, responsive to a transition from a first level to a second level towards achieving the endpoint. The transition may be determined based on the response data.

In some embodiments, the endpoint may be selected from a plurality of endpoints based on a mood indicated by the user. In some embodiments, the plurality of endpoints may be associated with at least one of a plurality of domains. The plurality of domains may include a social domain, a recreation domain, and a productivity domain. In some embodiments, the user may be presented with a comparison of a first rating performed prior to performance of the respective second activity and a second rating performed after performance of the respective second activity. In some embodiments, the configuration file may be selected based on a change in the endpoint determined using the response data from a previous time instance. In some embodiments, the one or more processors may determine to continue the repeating through the plurality of time instances based on an amount of time from the obtaining of the baseline metric. Repeating for each time instance of the plurality of time instances may include repeating the time instance responsive to determination to continue.

In some embodiments, repeating for each time instance of the plurality of time instances may include updating, for a time instance, the endpoint based on the response data identifying the one or more interactions by the user with the set of content items presented via an application from a previous time instance. In some embodiments, repeating for each time instance of the plurality of time instances may include converting human-readable instructions of the configuration file to generate a package including machine executable format instructions. In some embodiments, obtaining the first metric and the second metric may include obtaining the first metric and the second metric from a source separate from the application.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts a block diagram of a system for selecting configuration files for applications, in accordance with an illustrative embodiment;

FIG. 2A depicts a block diagram of a process for maintaining configuration files in the system for selecting configuration files, in accordance with an illustrative embodiment;

FIG. 2B depicts a block diagram of a process for assessing user profiles to provide configuration files in the system for selecting configuration files, in accordance with an illustrative embodiment;

FIG. 2C depicts a block diagram of a process for handling configuration packages in the system for selecting configuration files, in accordance with an illustrative embodiment;

FIG. 2D depicts a block diagram of a process for modifying user interfaces in the system for selecting configuration files, in accordance with an illustrative embodiment;

FIG. 2E depicts a block diagram of a process for assessing response data to provide configuration files in the system for selecting configuration files, in accordance with an illustrative embodiment;

FIG. 3 depicts a flow diagram of a method of selecting configuration files for applications, in accordance with an illustrative embodiment;

FIG. 4 depicts a block diagram of an architecture for a system for adaptive goal setting for selecting configuration files, in accordance with an illustrative embodiment;

FIG. 5A depicts a flow diagram of a method of performing adaptive goal setting in selecting configuration files, in accordance with an illustrative embodiment;

FIG. 5B depicts a flow diagram of a method of performing activities in accordance with the configuration files, in accordance with an illustrative embodiment;

FIGS. 6A-C each depict a block diagram of configuration files to perform routines, in accordance with an illustrative embodiment;

FIGS. 7A-F each depict examples of screenshots of a prompt to assess a physical activity of a user, in accordance with an illustrative embodiment;

FIGS. 8A-L each depict examples of screenshots of prompts to build a user profile in relation to an endpoint to pursue a hobby at a first step of level 0, in accordance with an illustrative embodiment;

FIGS. 9A-L each depict examples of screenshots of prompts for a user to perform activities associated with an endpoint to pursue a hobby at a second step of a level 0, in accordance with an illustrative embodiment;

FIGS. 10A-L each depict examples of screenshots of prompts for a user to perform activities associated with an endpoint to pursue a hobby at a third step of a level 0, in accordance with an illustrative embodiment;

FIGS. 11A-G each depict examples of screenshots of prompts for a user to perform activities associated with an endpoint to pursue a hobby at a fourth step of a level 0, in accordance with an illustrative embodiment;

FIGS. 12A-D each depict examples of screenshots of prompts for a user to perform activities associated with an endpoint to pursue a hobby at a first step of a level 1, in accordance with an illustrative embodiment;

FIGS. 13A-K each depict examples of screenshots of prompts for a user to perform activities associated with an endpoint to pursue a hobby at a second step of a level 1, in accordance with an illustrative embodiment;

FIGS. 14A-J each depict examples of screenshots of prompts for a user to perform activities associated with an endpoint to pursue a hobby at a third step of a level 1, in accordance with an illustrative embodiment;

FIGS. 15A-J each depict examples of screenshots of prompts for a user to perform activities associated with an endpoint to pursue a hobby at a third step of a level 1, in accordance with an illustrative embodiment;

FIGS. 16A-J each depict examples of screenshots of prompts for a user to perform activities associated with an endpoint to pursue a hobby at a fourth step of a level 1, in accordance with an illustrative embodiment;

FIGS. 17A-G each depict examples of screenshots of prompts for a user to indicate a mood of the user used to select next endpoints, in accordance with an illustrative embodiment;

FIGS. 18A-C each depict examples of screenshots of prompts for user to input personal values subsequent, in accordance with an illustrative embodiment;

FIGS. 19A-D each depict examples of screenshots of prompts for presenting pre-activity and post-activity assessments, in accordance with an illustrative embodiment;

FIG. 20 depicts a method of ameliorating experiential negative symptoms of schizophrenia in a user in need thereof in accordance with an illustrative embodiment;

FIG. 21 depicts a time diagram of a study schema for a multi-center, exploratory, single-arm study to evaluate the feasibility and acceptability of treatment with an abbreviated version of digital therapeutics application in adults diagnosed with schizophrenia;

FIG. 22 depicts a time diagram of a of a study schema for a combination therapy with digital therapeutics and antipsychotics in subjects with experiential negative symptoms of schizophrenia;

FIGS. 23A and 23B each depict a table of schedules of activities and assessments for participants; and

FIG. 24 is a block diagram of a server system and a client computer system in accordance with an illustrative embodiment.

### DETAILED DESCRIPTION

For purposes of reading the description of the various embodiments below, the following enumeration of the sections of the specification and their respective contents may be helpful:

Section A describes embodiments of systems and methods of selecting configuration files for applications;

Section B describes embodiments of methods ameliorating experiential negative symptoms of schizophrenia in subjects in need thereof; and

Section C describes a network and computing environment which may be useful for practicing embodiments described herein.

### A. System and Method for Selecting Configuration Files for Applications

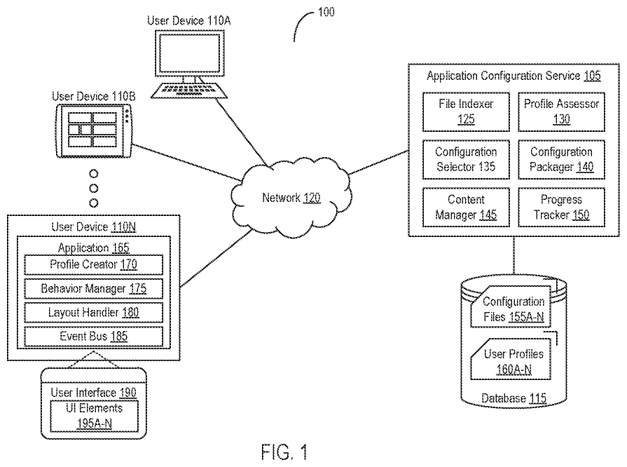

Referring now to FIG. 1, depicted is a block diagram of a system 100 for selecting configuration files for applications. In overview, the system 100 may include at least one application configuration service 105, one or more user devices 110A-N (hereinafter generally referred to as user device 110), and at least one database 115, communicatively coupled with one another via at least one network 120. The application configuration service 105 may include at least one file indexer 125, at least one profile assessor 130, at least one configuration selector 135, at least one configuration packager 140, at least one content manager 145, and at least one progress tracker 150, among others. The database 115 may store, maintain, or otherwise include a set of configuration files 155A-N (hereinafter generally referred to as configuration files 155) and a set of user profiles 160A-N (hereinafter generally referred to as user profiles 160), among others. At least one of the user devices 110 may include at least one application 165. The application 165 may include at least one profile creator 170, at least one behavior manager 175, at least one layout handler 180, and at least one event bus 185, among others. The application 165 may also provide at least one user interface 190 including one or more user interface elements 195A-N (hereinafter generally referred to as UI elements 195).

Each of the components in the system 100 (e.g., the application configuration service 105 and its components and each user device 110 and its components) may be executed, processed, or implemented using hardware or a combination of hardware, such as the system 1700 detailed herein in Section B. In some embodiments, the application configuration service 105 may be part of the user device 110 (e.g., as part of the application 165). In some embodiments, at least a portion of the functionalities of the application configuration service 105 (including the file indexer 125, the profile assessor 130, the configuration selector 135, the configuration packager 140, the content manager 145, and the progress tracker 150) may be performed on the user device 110. For example, the operations of the profile assessor 130, the configuration selector 135, and the configuration packager 140 may be performed on the user device 110.

In further detail, the application configuration service 105 may (sometimes herein generally referred to as a service) be any computing device comprising one or more processors coupled with memory and software and capable of performing the various processes and tasks described herein. The application configuration service 105 may be in communication with the one or more user devices 110 and the database 115 via the network 120. The application configuration service 105 may be situated, located, or otherwise associated with at least one server group. The server group may correspond to a data center, a branch office, or a site at which one or more servers corresponding to the application configuration service 105 is situated.

Within the application configuration service 105, the file indexer 125 may generate and store configuration files 155 to be provided to the applications 165. The profile assessor 130 may create user profiles 160 for users of applications 165 on user devices 110. The endpoint selector 135 may determine an endpoint for the user and select configuration files 155 to provide. The configuration packager 140 may provide the configuration files 155 to be loaded on the application 165. The content manager 145 may identify and provide content to be presented via the UI elements 195 of the user interface 190 for the application 165. The progress tracker 150 may update the user profiles 160 and manage re-determination of endpoints for the user. The functionalities of the various components of the application configuration service 105 may be carried out by the application 165 on the user device 110.

The user device 110 (sometimes herein referred to as a client, a client device, or an end user computing device) may be any computing device comprising one or more processors coupled with memory and software and capable of performing the various processes and tasks described herein. The user device 110 may be in communication with the application configuration service 105 and the database 115 via the network 120. The user device 110 may be a smartphone, other mobile phone, tablet computer, wearable computing device (e.g., smart watch, eyeglasses), or laptop computer. The user device 110 may be used to access the application 165. In some embodiments, the application 165 may be downloaded and installed on the user device 110 (e.g., via a digital distribution platform). In some embodiments, the application 165 may be a web application with resources accessible via the network 120.

The application 165 executing on the user device 110 may be a digital therapeutics application and may provide one or more sessions (sometimes referred to herein as a therapy session) to address at least one condition of the user. The condition of the user may include, for example, a habit (e.g., smoking, dieting, or exercising), a chronic pain (e.g., associated with or include arthritis, migraine, fibromyalgia, back pain, Lyme disease, endometriosis, repetitive stress injuries, irritable bowel syndrome, inflammatory bowel disease, and cancer pain), a skin pathology (e.g., atopic dermatitis, psoriasis, dermatillomania, and eczema), an affective disorder (e.g., depression, bipolar disorder, or dysthymia), a cognitive impairment (e.g., mild cognitive impairment (MCI), Alzheimer's, multiple sclerosis, and schizophrenia), and other ailments (e.g., narcolepsy and oncology), among others. The affective disorder and cognitive impairment may fall under a psychological (or mental) illness or disorder.

The user may be at least partially concurrently taking a medication to address the condition, while being provided sessions through application 120. The application 120 may increase the efficacy of the medication that the user is taking to address the condition. For instance, if the medication is for pain, the user may be taking acetaminophen; a nonsteroidal anti-inflammatory composition; an antidepressant; an anticonvulsant; or other composition; among others. For skin pathologies, the user may be taking a steroid, antihistamine, or topic antiseptic, among others. For cognitive impairments, the user may be taking cholinesterase inhibitors memantine, iclepertin, or an antipsychotic, such as risperidone, quetiapine, olanzapine, ziprasidone, paliperidone, or aripiprazole, among others. For a neurological disorder, the user may be taking a stimulant or antidepressant, among others. For affective disorders, the user may be on an antidepressant or mood stabilizers, among others. The user of the application 120 may also be undergoing other psychotherapies for these conditions.

The application 165 can include, present, or otherwise provide the user interface 190 including the one or more UI elements 195 to a user of the user device 110 in accordance with the configuration file 155 loaded on the application 165. The UI elements 195 may correspond to visual components of the user interface 190, such as a command button, a text box, a check box, a radio button, a menu item, and a slider, among others. The application 165 may be a digital therapeutics application and may provide a session (sometimes referred to herein as a therapy session) via the user interface 190 towards achieving one or more endpoints of the user (sometimes herein referred to as a patient, person, or subject).

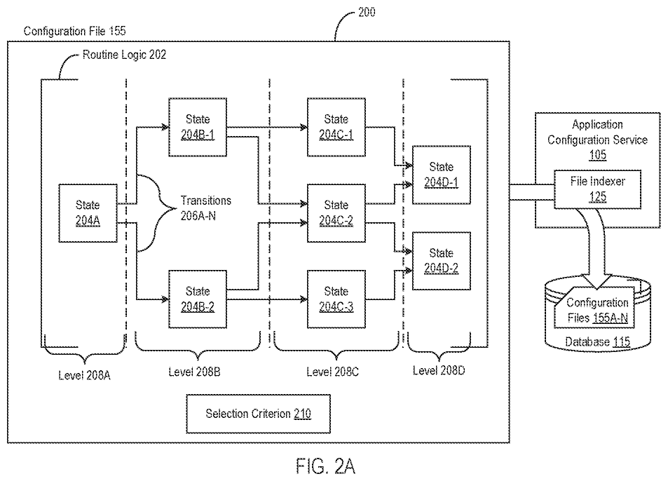

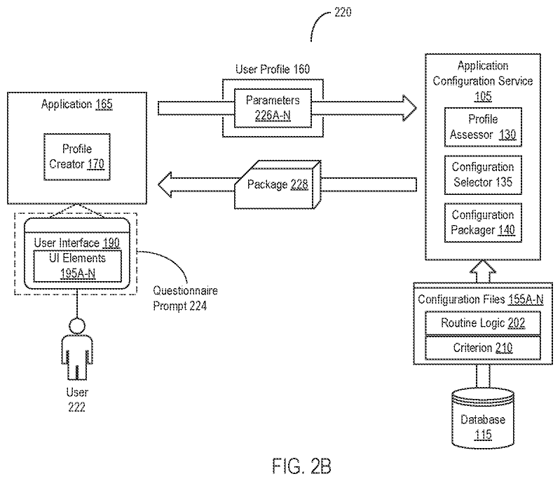

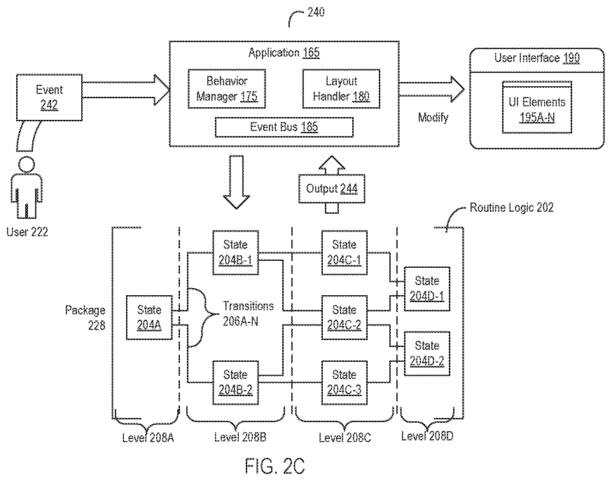

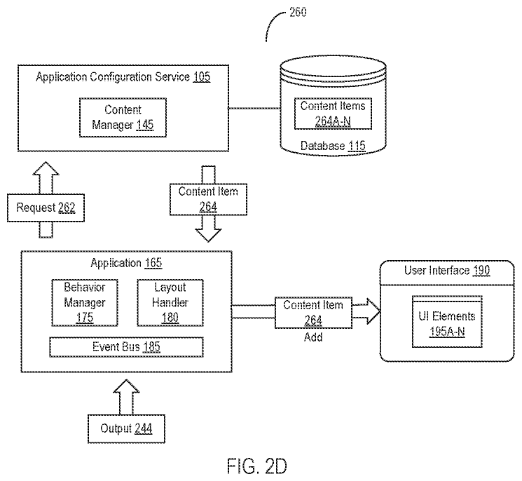

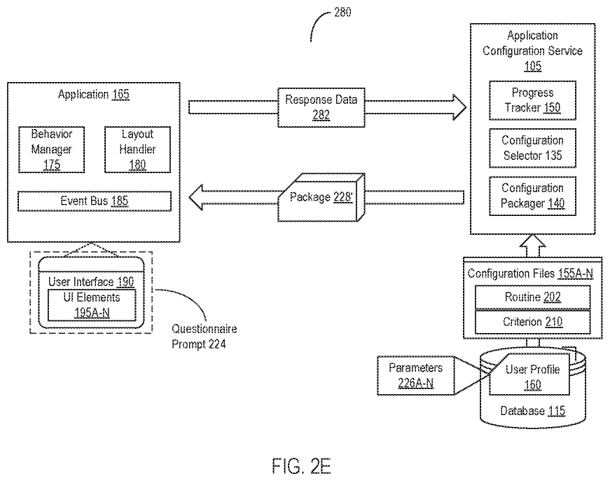

Referring now to FIG. 2A, depicted is a block diagram of a process 200 for maintaining configuration files in the system 100 for selecting configuration files. The process 200 may include or correspond to operations in the system 100 to store and catalogue configuration files for applications. Under the process 200, the file indexer 125 executing on the application configuration service 105 may retrieve, identify, or otherwise receive at least one configuration file 155. The configuration file 155 may be from another source, such as a computing device of a developer creating the configuration file 155. For example, a clinician may compose a script to form a suite of configuration files 155 for providing a digital therapeutic.

The configuration file 155 may include instructions for configuring, defining, or otherwise specifying various functionalities of a package (or plug-in) to be provided to the application 165. The configuration file 155 may include or correspond to one or more files include instructions defining a layout (e.g., presentation of UI elements 195 on the user interface 190) and behavior (e.g., functionality of the UI elements 195) of the application 165. The functionalities specified by the configuration file 155 may be separate from the built-in logic and functionalities of the application 165, including those of the application configuration service 105.

In some embodiments, the instructions included in the configuration file 155 may be in a human-readable format, such as Yet Another Markup Language (YAML), Extensible Markup Language (XML), and JavaScript Object Notation (JSON), among others. The format of the instructions of the configuration file 155 may be a human-readable data serialization language to define structured data. The format may be different from binary formats that can be read and executed by one or more processors running the application 165. In this manner, the effort undertaken by a developer in writing the human readable instructions in the configuration file 155 may be less than that of composing instructions in other formats (e.g., a higher-level programming language, such as Java, C++, or python). Furthermore, since the configuration file 155 specifies custom functionality for the application 165 without modifying the underlying code for the application 165, configuration files 155 may be loaded and interchanged with one another by the application 165, thereby expanding the functionality of the application 165.

The configuration file 155 may specify, define, or otherwise identify at least one routine logic 202. In some embodiments, the routine logic 202 itself may be defined in accordance with an automaton, such as a finite state machine (FSM), a decision tree, or a pushdown automaton, among others. The routine logic 202 may identify a set of content items to prompt users to perform a set of activities towards achieving one or more endpoints (sometimes referred herein as goals, targets, or objectives) in addressing a condition of a user of the application 165. For example, the condition may include smoking, obesity, psychological disorders (e.g., Schizophrenia), mental cognition, and depression, among others, on the part of the user. The activities defined by the routine logic 202 may include those for treatment or management of the conditions. The set of activities may form steps along a progression of a user to achieve the target endpoint for a given condition, such as cessation of smoking.

The routine logic 202 of the configuration file 155 may identify or include a set of states 204A-N (hereinafter generally referred to as states 204), a set of transitions 206A-N (hereinafter generally referred to as transitions 206), and a set of levels 208A-N (hereinafter generally referred to as levels 208), among others. The set of states 204, the set of transitions 206, and the set of levels 208 in conjunction may be used to define or specify the set of activities to be performed by the user of the application 165 towards achieving one or more endpoints. Each state 204 may define, identify, or otherwise specify at least one output to be produced by the routine logic 202 upon invocation. The output may be for a particular activity for the set of activities and may include one or more operations to be performed by the application 165. In some embodiments, the output may identify user interface elements 195 to be presented via the user interface 190 of the application 165. For example, the output may include or specify a set of content items to be generated or retrieved (e.g., using one or more identifiers (e.g., uniform resource locator (URL))) for presentation as user interface elements 195 in the user interface 190. In some embodiments, the output may identify another configuration file 155 to be loaded. For instance, the output may include an identifier (e.g., a file name) for the next configuration file 155 to be loaded on the application 165. At least one state 204 may correspond to the state 204 at which the routine logic 202 is to start from (e.g., the state 204A as depicted).

Between a pair of states 204, the routine logic 202 may define, specify, or otherwise include at least one transition 206. Each transition 206 may define, identify, or otherwise specify an event to be detected via the application 165 to transition or update the routine logic 202 from one state 204 to another state 204. The event may correspond to a user interaction received via the application 165 on the user device 110. For instance, the transition 206 may specify to move from one state 204A to the next state 204B, the user is to record completion of Exercise A via the user interface 1685 of the application 165. In some embodiments, the routine logic 202 may include at least two transitions 206 from a given state 204. For example, the routine logic 202 may identify one transition 206 for successful completion of the activity associated with the state 204. Conversely, the routine logic 202 may identify another transition 206 for failure of completion of the activity associated with the state 204.

Among the set of states 204 and the set of transitions 206, the routine logic 202 may define, identify, or otherwise include the levels 208. In some embodiments, the level 208 may be defined, identified, or otherwise specified in the states 204 or the transitions 206 themselves. Depending on the level 208, the set of activities specified by the corresponding set of states 204 may be defined in terms of a duration, a frequency, and a time of day for the activity. In some embodiments, the levels 208 may correspond to the same endpoint or a different endpoint. For example, the first level 208A may correspond to a lower level for a given endpoint and the second level 208B may correspond to a higher level for the same endpoint or another endpoint. In general, with the successful completion of the activity from the previous state 204, the higher the level 208, the activity associated with a given state 204 may have a higher difficulty or intensity in terms of duration or frequency, or both. For example, for a set of activities, one state 204A at a lower level 208A may specify Activity B once a week in the evening, while another state 204B-1 at a higher level 208B may specify Activity C five times a week every morning. Conversely, with the failure in completion of the activity from the previous state 204, the higher the level 208, the activity associated with the given state 204 may lower difficulty or intensity in terms of duration or frequency, or both. The activity associated with the given state 204 may differ from the activity associated with the previous state 204. For example, the activity may be part of a mitigation measure for when the previous activity is not successfully completed.

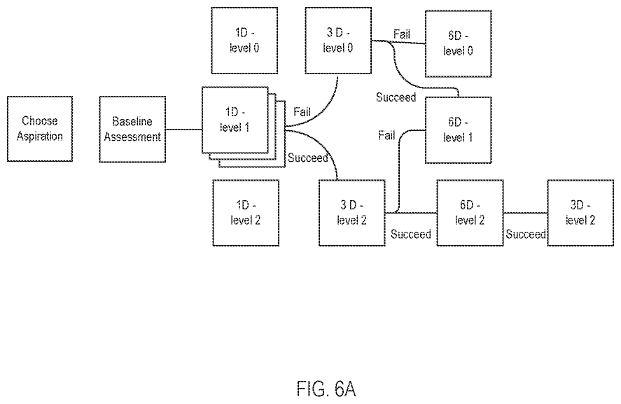

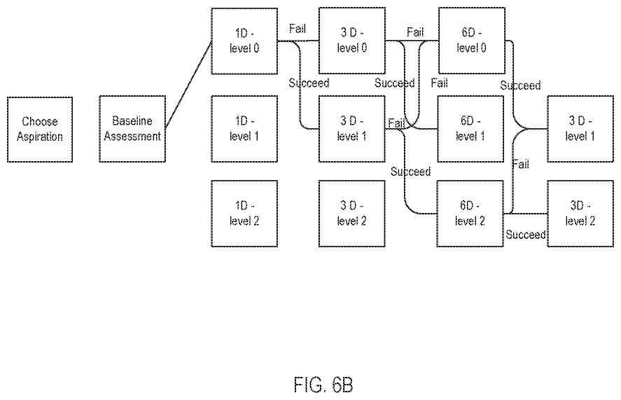

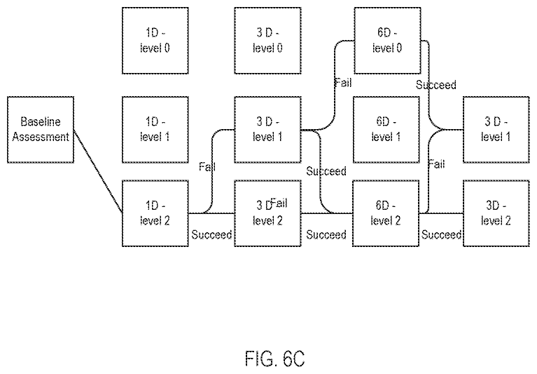

With the definition of the states 204, the transitions 206, and the levels 208, the routine logic 202 of the configuration file 155 may titrate the type, frequency, and duration of activity to specify the user to perform via the application 165. Furthermore, the routine logic 202 may adaptively produce outputs in response to the actions of the user of the application 165. Examples of the routine logic 202, including the states 204, the transitions 206, and the levels 208, are detailed herein in conjunction with FIGS. 6A-C. The routine logic 202 across the set of configuration files 155 may be used to effectuate an adaptive goal setting (AGS) framework. For example, as depicted in FIGS. 6A-C, the user may take a baseline assessment and select an aspiration. The user may then be presented with a 1-day (1D) goal (or endpoint) to orient the user. The user may then proceed to a 3-day (3D) goal at the same level or lower level depending on success. The user may progress through the 3-day goals until reaching the lowest or highest level. The user may be able to choose a new aspiration to work on (if there is sufficient time remaining).

Each configuration file 155 may correspond to one or more goals and may be selected based on a baseline assessment of the user's function to inform the level of the goal and associated activities. An assessment of the user's interests or aspirations in the domain may be used to identify the content of the goal and associated activities for the goal and configuration files 155. Following the introduction, the users can receive up to any number of goals as part of the configuration file 155. For example, if there are four phases, a user might receive 4 goals, then 4 more goals, then 4 more goals, then 8 goals. Each goal may be associated, for example, with four daily activities. Users that complete all activities associated with the goal may transition onto the next level goal within the same interest or aspiration. When the user completes a goal, the application 165 may provide a positive affirmation as well as opportunities to savor positive emotions associated with completing their goal. If the minimum or maximum goal level within an interest or aspiration has been reached, the new goal may be within a new interest or aspiration presented via the application 165 at the level determined from the initial baseline assessment. If a certain number of goals within the same interest or aspiration has been reached, the user may be asked to reselect an interest or aspiration.

When provided to the application 165, the configuration file 155 may provide one or more endpoint or goal-related activities for a user of the application 165 to perform and complete. The configuration file 155 may provide the user the option (e.g., via user interface elements 195 on the user interface 190 of the application 165) to perform the specified activities when prompted or schedule at a later time. The activities may be configured to increase likelihood of reflection and practice of therapeutic skills learned through the activities. The configuration files 155 and the activities may be selected based on personal values related to classifications of endpoints.

Through selective provision of the configuration files 155, the application 165 may provide daily activities within an endpoint that are sequential. When the user completes an activity, the application 165 may provide a positive affirmation. If the user indicates during daily check-in that they did not complete the previous day's activity, the application 165 may prompt the user to perform the same daily activity to continue towards achieving the endpoint or goal. New activities may be locked if the user has been inactive or has not completed the preceding activity. For example, after a certain time period (e.g., 1-2 days) of inactivity or activity non-completion, the user may be prompted by the application 165 with the same daily activity to continue. After another period of time (e.g., above 3 days) of inactivity or activity non-completion, the user may be provided via the application 165 the option of repeating the previously presented activity or getting an activity from a new endpoint.

Continuing on, for certain phases, if a user chooses to get an activity from a new endpoint, the activity may be presented at a decremented level within the same aspiration via the application 165. If the user has reached the lowest possible level for an endpoint, the user may be presented with repeating the previously presented activity. For other configuration files 155, if the user selects an activity from a new endpoint, the user may be presented to reselect an aspiration or domain. After a daily activity is presented to the user, the application 165 may prompt the user to either start the activity immediately or set a time later in the day to complete the activity. If the user chooses to set a reminder, the application 165 may send a notification at the selected time. The user may have the option to start the scheduled activity ahead of or after the scheduled time via the home screen of the application 165.

In addition, the configuration file 155 may identify, define, or specify at least one selection criterion 210 for selection of the corresponding configuration file 155. In some embodiments, the selection criterion 210 may be separate from the configuration file 155 and be associated with the configuration file 155. For example, an association between the selection criterion 210 and the configuration file 155 may be stored on the database 115 using one or more data structures, such as a linked list, a tree, a table, an array, a graph, a heap, or hash tables, among others. The selection criterion 210 may specify one or more parameters identified with the user profile 160 for which the configuration file 155 is to be selected for provision to the application 165 on the user device 110. When the parameters specified in the selection criterion 210 match the parameters identified in the user profile 160, the configuration file 155 may be selected for provision to the application 165. In some embodiments, the selection criterion 210 may define one or more baseline measures derived from the user profile 160 for which the configuration file 155 is to be selected. When the measures derived from the parameters of the user profile 160 satisfy (e.g., greater than or equal to) the baseline measures specified by the selection criterion 210, the configuration file 155 may be selected for provision to the application 165. Additional details in relation to the parameters or baseline measures for the selection criterion 210 will be detailed herein below.

Upon receipt, the file indexer 125 may store and maintain the configuration file 155 on the database 115. The database 115 may be part of the application configuration service 105 or otherwise be accessible by the application configuration service 105. In some embodiments, the file indexer 125 may store an association between the configuration file 155 and the source from which the configuration file 155 is received. In some embodiments, the file indexer 125 may store an association between the configuration file 155 and a version identifier for the configuration file 155. The configuration file 155 may be maintained on the database 115 for provision to instances of the applications 165 across various clients 110. As the configuration file 155 is separate from the application 165, the configuration files 155 may be readily updated and interchanged.

Referring now to FIG. 2B, depicted is a block diagram of a process 220 for assessing user profiles to provide configuration files in the system 100 for selecting configuration files. The process 220 may include or correspond to operations in the system 100 for evaluating parameters in user profiles to select configuration files 155 to generate packages for provision to the application 165. Under the process 220, the profile creator 170 of the application 165 executing on the user device 110 may produce, output, or otherwise generate the user profile 160 for a user 222 of the application 165. The user profile 160 may be generated based on one or more responses from the user 222. The responses may identify, define, or otherwise be associated with the endpoint and the condition as indicated by the user 222.

In generating the user profile 160, the profile creator 170 may display, render, or otherwise present at least one prompt 224 to receive the responses from the user 222. The prompt 224 may be presented to the user 222 using one or more of the user interface elements 195 on the user interface 190. In some embodiments, the prompt 224 may be presented upon installation of the application 165 on the user device 110. In some embodiments, the prompt 224 may be presented in response to an interaction on the user interface 190 on the application 165. The interaction may correspond to a request by the user 222 for another endpoint or a new set of activities to be performed via the application 165. In some embodiments, the prompt 224 may be in accordance with a defined time period, for example, once every 2-6 hours, once a day in the morning, or once every week, among others. The prompt 224 may identify or include a set of questions for the user 222. The questions themselves may correspond to text, audio, or visual content on the user interface elements 195 of the user interface 190. The responses to the questions may be entered or inputted by the user 222 via other user interface elements 195, such as radio buttons, command buttons, text boxes, sliders, or check boxes, among others.

The profile creator 170 may include one or more event listeners to detect, retrieve, or otherwise receive responses via the prompt 224 presented on the user interface 190. The set of questions presented in the prompt 224 may be composed, set, or otherwise configured by the administrator of the application configuration service 105 or the application 165. The questions may ask the user 222 to indicate at least one desired endpoint to achieve (sometimes herein referred to as a goal, objective, or target) and at least one condition to be addressed (e.g., behavioral, psychological, or physical), among others. The questions may also ask the user 222 to identify at least one state (e.g., mood, emotional, behavioral, or physiological state) of the user 222. The questions may also ask the user 222 preferences for performance of activities or routines, such as type of routine, frequency, duration, day of week, and time of day, among others. The responses to the questions may be recorded, inputted, or entered via the user interface elements 195 on the user interface 190.

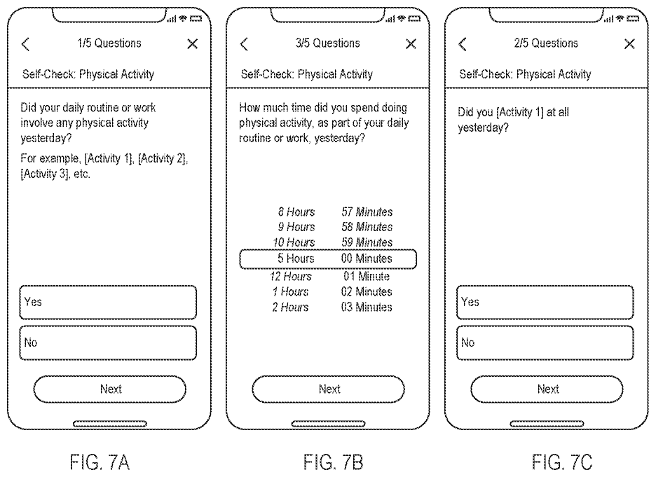

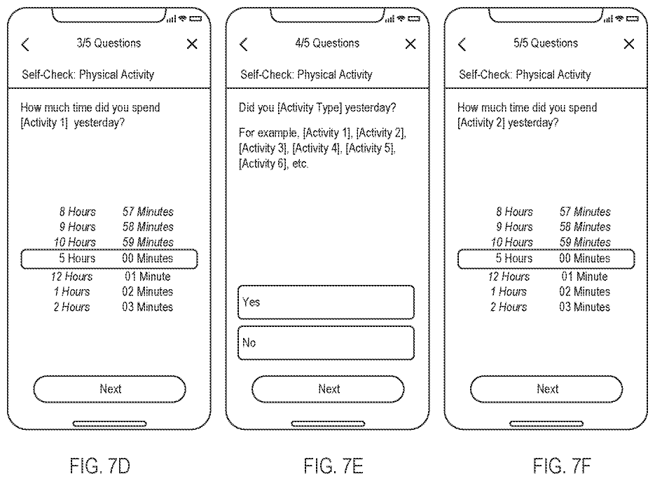

In some embodiments, the set of questions to be presented by the questionnaire prompt 224 may be in accordance with a questionnaire policy. The questionnaire policy may identify a rule for selecting questions depending on the responses to previous questions within the set. The responses to the questions may be used to generate the user profile 160 for the user 222. For example, the questionnaire may specify presentation of a subset of questions on frequency, duration, and level for the hobby of Hobby A, when the user 222 has responded a preference for Hobby A as the type of activity to be performed and recorded via the application 165. In some embodiments, the questionnaire may be a validated clinical assessment in relation to a physical or psychological (or mental) condition, such as the Structured Clinical Interview for the Diagnostic and Statistical Manual of Mental Disorders (DSM) (SCID) to assess diagnoses from the DSM; Mini-international neuropsychiatric interview (MINI); the clinical assessment interview for negative symptoms (CAINS) scale, which assesses the motivation and pleasure domain of negative symptoms; and Hamilton Depression Rating Scale (HDRS), among others. Examples of the prompt 224 and the set of questions presented therein are shown herein in FIGS. 7A-F (related to physical activity of the user). In the depicted example, the questionnaire policy may specify that the prompt 224 is to present a question on how much time the user spent in performing a physical activity (FIG. 7B), when the answer to the previous question on whether the physical activity was performed is in the affirmative (FIG. 7A).

Using the responses to the questions presented via the prompt 224, the profile creator 170 may determine or generate one or more parameters 226A-N (hereinafter generally referred to as parameters 226). The parameter 226 may identify or include any number of factors to be used to select one or more configuration files 155 to be provided to the application 165. The parameters 226 may identify the endpoint to be achieved and the condition of the user 222 to be addressed. The parameters 226 may identify the state of the user 222 (e.g., mood, emotional, behavioral, physiological state). The parameters 226 may identify a type of activity, a frequency, duration, and time (e.g., day of week or time of day) for the routine, among others, as indicated by the user 222 via the prompt 224. In some embodiments, the parameters 226 may identify an identifier for the user device 110, a device type of the user device 110, an identifier for the application 165, and a location of the user 222, among others. With the generation, the profile creator 170 may include the one or more parameters 226 into the user profile 160. The inclusion of the user profile 160 may be in accordance with a template. The template may include fields in which to include or insert the values of the parameters 226. The profile creator 170 may send, transmit, or otherwise provide the user profile 160 to the application configuration service 105. The user profile 160 may be sent as part of a message to the application configuration service 105.

The profile assessor 130 executing on the application configuration service 105 may retrieve, receive, or otherwise identify the user profile 160 from the user device 110. In some embodiments, the profile assessor 130 may be executing on the user device 110 as part of the application 165, for example, to evaluate the user profile 160 generated by the profile creator 170 on the user device 110. Upon receipt from the user device 110, the profile assessor 130 may store and maintain the user profile 160 on the database 115. In some embodiments, the profile assessor 130 may fetch, retrieve, or otherwise identify the user profile 160 from the database 115. The user profile 160 associated with the user 222 of the user device 110 may be stored and maintained on the database 115. The user profile 160 maintained on the database 115 may be updated using instances of the user profile 160 received from the user device 110. With the identification, the profile assessor 130 may parse the user profile 160 to extract or identify the one or more parameters 226 in the user profile 160.

Using the parameters 226, the profile assessor 130 may calculate, determine, or generate one or more measures for the user 222 associated with the user profile 160. The measures derived from the parameters 226 of the user profile 160 may be used for selecting configuration files 155 to provide to the application 165. The generation of the measures may be in accordance with a function of the parameters 226. For example, the parameters 226 may include user responses to the questions depicted in FIGS. 7A-D, and may be related to physical activity, such as whether the user 222 had performed a physical activity and how much time the user 222 had spent in performing the physical activity. In this example, the profile assessor 130 may generate measures related to a physical activity for the user 222, such as a level of physical activity.

In some embodiments, the profile assessor 130 may calculate, determine, or otherwise generate at least one measure for the user profile 160 that may be based on the user responses to the clinical assessment questionnaire, such as SCID, MINI, CAINS, and HDRS, among others. The generation of the measure may be in accordance with a function for the clinical assessments. The measure may identify a characteristic of the condition, such as a severity of the condition. When determined prior to the performance of any activity, the measure may be determined as a baseline assessment measure for the user 222. When determined after one or more activities, the measure may be determined as an assessment measure for the user 222.

In some embodiments, the profile assessor 130 may generate the measure to identify a degree of a characteristic of the condition of the user 222. The characteristics of the user 222 may include, for example, a severity of a behavioral condition, a physical condition, or a psychological (or mental) condition, among others. In some embodiments, the measure may identify a likelihood that the user 222 is to perform the type of activity when prompted via the application 165. For instance, the measure may indicate a likelihood that the user 222 will perform a hobby and another measure may indicate a likelihood that the user 222 will carry out an exercise when prompted via the user interface 190 of the application 165. In some embodiments, the measure may identify a predicted efficacy of the type of activity towards achieving one or more endpoints. For example, the measure may identify how effective performance of a run would be to addressing the user's condition related to improving diet, which the user has identified as a desired endpoint.

In addition, the profile assessor 130 may store and maintain the user profile 160 including the parameters 226 on the database 115. In some embodiments, the profile assessor 130 may store and maintain the measures derived from the parameters 226 in the database 115. In some embodiments, the profile assessor 130 may include the measures in the user profile 160. In some embodiments, the profile assessor 130 may generate an association between the measures and the user profile 160 using one or more data structures. With the generation, the profile assessor 130 may store and maintain the association on the database 115. In some embodiments, the profile assessor 130 may perform the functionalities described herein in conjunction with the profile creator 170, and vice-versa.

The configuration selector 135 executing on the application configuration service 105 may select, identify, or otherwise determine one or more endpoints to address the condition of the user 222. In general, the endpoints may correspond to an outcome or measure associated with addressing the condition of the user 222. The endpoint may correspond to a mark of completion of a defined set of activities aimed at addressing the condition of the user 222. For example, the endpoint may be associated with a set number of activities over a defined window of time (e.g., 4 activities per day), and the completion of the activities by the user 222 may correspond to an attainment of the endpoint. The endpoint may also correspond to a measured improvement to the condition of the user 222. For instance, the endpoint may be associated with a perceived decrease in a sense of pain (or other metric) associated with the condition of the user 222.

In some embodiments, the endpoints may be categorized into one or more classifications (sometimes referred herein as domains). For instance, one set of endpoints may be associated with a first classification and another set of endpoints may be associated with a different second classification. Each endpoint may be associated with a set number of activities to accomplish, for example, four activities. Each domain may be associated with a set number of endpoints, for example, four endpoints. Each domain may be associated with a set number of parameters indicated by the user 222, for example, three aspirations. The endpoints may include, for example, a social domain, a recreation domain, or a productivity domain, among others. These domains may be associated with addressing a particular condition. A subset of endpoints associated with the social domain may be directed to improving sociality of the user. Another subsets endpoints associated with the recreation domain may identify activities for performing leisurely exercises or tasks for the user. Another subset endpoints associated with the productivity domain may include activities for improving attention or work habits of the use.

The configuration selector 135 may determine the endpoint for the user 222 based on the condition to be addressed for the user 222. For instance, when the condition indicated by the user 222 is dieting, the configuration selector 135 may select one or more endpoints related to dieting, such as eating a snack, walking, or jogging, among others. In some embodiments, the configuration selector 135 may determine the endpoint based on at least one activity (or type of activity) indicated by the user 222 towards achieving the endpoint. In some embodiments, the configuration selector 135 may determine the endpoint based on the parameters 226 of the user profile 160. For example, the configuration selector 135 may determine the endpoint using the type of activity, a frequency, duration, and time as identified by the user 222. In some embodiments, the configuration selector 135 may determine the endpoint based on the measure (e.g., baseline assessment metric for clinical assessment, likelihood that the user is perform the activity, or the predicted efficacy) from the clinical assessment.

In some embodiments, the configuration selector 135 may select or identify one or more activities associated with the endpoint to be performed by the user 222 via the application 165. The identification of the activities may be based on the parameters 226 as indicated by the user 222. For instance, the configuration selector 135 may identify the activities such as Activity D, Activity E, or Activity F when the user 222 indicates the desired types of activities. In some embodiments, the configuration selector 135 may be executing on the user device 110 as part of the application 165, for example, to identify the endpoints for the user 222 of the user device 110. The identification of the endpoints may be based on the parameters 226 of the user profile 160 and the selection criterion 210 for each endpoint. As discussed, the selection criterion 210 may specify parameters 226 for which the configuration file 155 associated with the endpoint is to be selected.

The configuration selector 135 may identify or select one or more configuration files 155 from the overall set of configuration files 155 to provide to the application 165 on the user device 110. Based on the endpoint (or activities towards achieving the endpoint), the configuration selector 135 may select the associated configuration file 155 for provision to the application 165. In some embodiments, the configuration selector 135 may select multiple configuration files 155 for at least one endpoint. For example, when the determined endpoint is for addressing smoking cessation, the configuration selector 135 may identify a set of configuration files 155 marked as addressing smoking cessation in the selection criterion 210. Conversely, for each routine identified as to not be included, the configuration selector 135 may refrain from selecting the associated configuration file 155 for provision to the application 165.

To select, the configuration selector 135 may identify the selection criterion 210 for each configuration files 155 from the database 115. In some embodiments, the configuration selector 135 may parse each configuration file 155 to extract or identify the selection criterion 210 for the corresponding endpoint. In some embodiments, the configuration selector 135 may identify the selection criterion 210 for the specified one or more activities. With the identification, the configuration selector 135 may compare the parameters 226 with the selection criterion 210 for the endpoint. When the parameters 226 match with the parameters specified by the selection criterion 210, the configuration selector 135 may select the configuration file 155. Otherwise, when the parameters 226 do not match with the parameters specified by the selection criterion 210, the configuration selector 135 may refrain from selecting the configuration file 155.

In some embodiments, the routine selector 315 may compare the measures derived from the parameters 226 with the selection criterion 210 for the routine to determine whether to select the given configuration file 155. When the determined measures satisfy (e.g., greater than or equal to, or within) the measures specified by the selection criterion 210, the routine selector 315 may select the configuration file 155 for provision. Otherwise, when the parameters 226 do not satisfy (e.g., less than or outside) the parameters specified by the selection criterion 210, the routine selector 315 may refrain from selecting the corresponding configuration file 155 for provision.

With the selection, the configuration selector 135 may relay, convey, or otherwise provide an identification of the selected configuration files 155 (or by extension, routines) to the profile assessor 130 to set or update the user profile 160. Stored in the database 115, the user profile 160 may be used to keep track of the routines and configuration files 155 provided to the user 222 and the progress of the user 222 in each routine using the state 204 and the level 208 of the routine logic 202. When storing the user profile 160 as part of the initial selection of the configuration file 155, the profile assessor 130 may identify the state 204 and the level 208 for each selected routine as in the initial state (e.g., the state 204A) and initial level (e.g., the level 208A) respectively. The profile assessor 130 may store and maintain the initial state 204 and the initial level 208 for each selected configuration file 155 in the user profile 160 on the database 115.

The configuration packager 140 executing on the application configuration service 105 may produce, output, or otherwise generate at least one package 228 using the selected configuration files 155. In some embodiments, the configuration packager 140 may be executed on the user device 110, for example, to generate the package 228 to be loaded by the application 165. The package 228 may include instructions for configuring, defining, or otherwise specifying various functionalities to be performed on the application 165. In general, the instructions in the package 228 may be in machine-readable code. The instructions for the package 228 may also define the routine logic 202, including the set of states, the transitions 206, and the levels 208. In addition, the instructions for the package 228 may also define the user interface elements 195 for the outputs in the states 204 in the routine logic 202. Because the package 228 is generated separately from the application 165, which packages 228 the application 165 is to include may be readily interchanged, based on the condition of the user to be addressed.

In generating, the configuration packager 140 may create a separate file in which to store the specifications for the package 228. The configuration packager 140 may parse the one or more configuration files 155 to read or identify the instructions in the original format. In some embodiments, in generating the package 228, the configuration packager 140 may insert, add, or otherwise include information from the user profile 160 into the package 228. For instance, the configuration package 140 may populate the state 204, the level 208, and the name of the user 222 into fields in the package 228 while generating. The included information may be presented via the user interface elements 195 when the package 228 is loaded onto the application 165.

Upon identification of the configuration file 155, the configuration packager 140 may generate or determine an equivalent instruction in an executable format for inclusion into the application 165. In some embodiments, the configuration packager 140 may compile the configuration file 155 to generate instructions in a lower-level language. When compiled, the instructions for the package 228 may be in a lower-level language, such as in binary, byte code, assembly, object code, or machine code, among others, to be read by processors executing the application 165. The instructions in the lower-level language for the package 228 may differ from the human-readable instructions in the configuration file 155, in that the instructions 228 are readable and processable by the processor running the application 165. In some embodiments, the configuration packager 140 may transpile the configuration file 155 to generate instructions in an intermediate-format to be added to the application 165. For example, the instructions may be in a similar-level language as the original of the configuration file 155, such as JavaScript or Typescript, among others. Upon generation, the configuration packager 140 may write the equivalent instruction into the file for the package 228 and may repeat until the end of the configuration file 155.

With the generation, the configuration packager 140 may provide the instructions for the package 228 to add or include to the application 165. In some embodiments, the configuration packager 140 may insert or inject the package 228 into the application 165, prior to provision to the user device 110. For example, the configuration packager 140 may inject the package 228 into the application 165 that already includes the other components, such as the behavior manager 175, the layout handler 180, and the event bus 185, among others. The configuration packager 140 may provide the application 165 containing the injected package 228 to the user device 110 via a digital distribution platform (e.g., application market or store). The user device 110 may request to download or retrieve the application 165 from the application configuration service 105 (or the digital distribution platform) for installation. Once received, the user device 110 may unpack and install the application 165 including the package 228.

The configuration packager 140 may send, transmit, or otherwise provide the instructions for the package 228 to add or include the application 165 that is installed on the user device 110. For example, the user device 110 may have previously installed the application 165 received from the application configuration service 105 (e.g., via the digital distribution platform). In some embodiments, the user device 110 may subsequently request an update of the configuration of the application 165. In some embodiments, the configuration packager 140 may identify or determine that an update is to be provided to the application 165 through the configuration file 155. For instance, a system administrator of the application configuration service 1605 may direct that instances of the application 165 are to be updated. With the identification of the update, the configuration packager 140 in turn may provide the instructions for the package 228, without providing the other components of the application 165. Upon receipt, the user device 110 (or the application 165 itself) may update the already installed application 165 to include the package 228. In some embodiments, the received instructions for the package 228 may be in the intermediary format, and the application 165 may further compile the instructions to generate the lower-level format to run on the user device 110.

Referring now to FIG. 2C, depicted is a block diagram of a process 240 for handling configuration packages in the system 100 for selecting configuration files. The process 240 may include or correspond to the operations performed in the system 100 upon loading and running the routine logic 202 as defined in the package 228. Under the process 240, the application 165 (or an application service of the application 165) may perform initialization operations, such as starting the execution of the behavior manager 175, the layout handler 180, the event bus 185, and the user interface 190, among others. The application 165 may run various logic and operations defined for the application 165 outside of the package 228 received from the application configuration service 105. The application 165 may retrieve, identify or otherwise receive the package 228 from the application configuration service 105.

The behavior manager 175 of the application 165 executing on the user device 110 may parse the package 228 to read, load, and run the routine logic 202. As described above, the routine logic 202 may correspond to a set of activities to be performed, recorded, and logged by the user 222 via the application 165 on the user device 110. In some embodiments, the behavior manager 175 may parse multiple packages 228 to run multiple, corresponding routing logics 202. For each routine logic 202, the behavior manager 175 may keep track of the current state 204 of the routine logic 202. In some embodiments, the behavior manager 175 may use or maintain an identifier for the current state 204 for each routine logic 202 to keep track. In addition, the behavior manager 175 may keep track of the current level 208 for the user 222 as defined in the routine logic 202. In some embodiments, the behavior manager 175 may identify the current level 208 from the current state 204 at which the routine logic 202 is in. Upon initialization, the current state 204 of the routine logic 202 may correspond to the starting state 204 (e.g., state 204A in the depicted example). The current level 208 may correspond to the first level 208 (e.g., the level 208A in the depicted example).

In conjunction, the behavior manager 175 may monitor or listen for at least one event 242 on one or more of the user interface elements 195 in the user interface 190 via the event bus 185. The event 242 may correspond to activity performed by a user 222 of the application 165. For example, the user interface 190 may present a prompt for the user 222 to conduct Exercise B, and the user 222 may indicate the completion of the exercise via interaction with one of the user interface elements 195 on the user interface 190. The event 242 may thus correspond to a completion of the activity as specified by the state 204 in the routine logic 202.

In some embodiments, the behavior manager 175 may monitor or listen for the event 242 from another process of the application 165 or user device 110. The event 242 in this case may correspond to an occurrence of an action by a process of the application 165 or the user device 110 that was not triggered by an interaction from the user 222. For example, the behavior manager 175 may receive time elapsed from the presentation of the prompt to conduct the activity via a system timer on the user device 110. The behavior manager 175 may compare the elapsed time with the timespan specified by the state 204 within which to complete the activity. The behavior manager 175 may identify the exceeding of the specified time as the event 242.

In some embodiments, the behavior manager 175 may prompt the user 222 to select or identify whether to perform the activity presented in the prompt. For example, after receiving their activity prompt, the user 222 may select to either “do it now” or “do it later” to increase the likelihood of completion through multiple paths. The former option may allow the user 222 to indicate the user 222 is behaviorally activated. This may be a major benefit by being able to capture users when they are motivated, as compared to a different, more fixed setting that only permits the latter option. The latter option may reinforce the behavior to plan for an activity, select a time, and receive a reminder, and do the activity with the application 165 at a specified later time. When the identification is to perform the activity at a later time, the behavior manager 175 may store the indication, and may re-present the prompt for the activity at the specified time. Otherwise, when the indication is to perform the activity, the behavior manager 175 may proceed with further processing to load the routine logic 202 of the package 228.

Based on the detection of the event 242, the behavior manager 175 may determine or select at least one routine logic 202 in the corresponding package 228 to invoke. In some embodiments, the behavior manager 175 may convey or pass the detected event 242 to the package 228 via the event bus 185. The event bus 185 may correspond to an interface between the package 228 and the various components of the application 165, such as the behavior manager 175 and the layout handler 180, among others. By passing, the behavior manager 175 may check the detected event 242 against the event specified by the transitions 206 for the current state 204. As discussed above, the behavior manager 175 may keep track of the current state 204 and the current level 208 of each routine logic 202 in the respective package 228. In some embodiments, the behavior manager 175 may detect or receive the result of the checking of the detected event 242 via the event bus 185.

From the routine logic 202, the behavior manager 175 may identify the specified event for each transition 206 associated with the current state 204 to check against the detected event 242. When the detected event 242 does not correspond to the specification in any of the transitions 206 of the current state 204, the behavior manager 175 may maintain the routine logic 202 at the current state 204 and the current level 208. In some embodiments, the behavior manager 175 may also refrain from invoking the routine logic 202. The maintenance of the routine logic 202 at the current state 204 may correspond to the user 222 not having completed an activity of the routine set out for the routine logic 202 for any of the transitions 206 associated with the current state 204. The behavior manager 175 may continue to check the detected event 242 against the specifications of the routine logic 202 in other packages 228.

Conversely, when the detected event 242 corresponds to the specification in one of the transitions 206 of the current state 204, the behavior manager 175 may select the routine logic 202 to invoke. By invoking, the behavior manager 175 may update the current state 204 and the current level 208 of the routine logic 202 to the next state 204 in accordance with the transition 206. The updating of the routine logic 202 from the current state 204 to the next state 204 may correspond to the user 222 having completed (successfully or unsuccessfully) an activity of the routine set out for the routine logic 202 as identified for at least one of the transitions 206 associated with the current state 204. For example, the current state 204 may be updated from the initial state 204A to the state 204B-1 upon successful completion as specified by the respective transition 206. In contrast, the current state 204 may be transitioned from the initial state 204A to the state 204B-2, upon failure of completion as defined by the respective transition 206.

In addition, from invoking the routine logic 202 of the package 280, the behavior manager 175 may retrieve or identify an output 244 identified by the next state 204 of the routine logic 202. The output 244 may be produced or generated by the state 204 as specified in the routine logic 202 upon invocation. The output 244, as discussed above, may identify the user interface elements 195 to be presented via the user interface 190 of the application 165. In some embodiments, the output 244 may specify modifications to be applied to the user interface elements 195 of the user interface 190. The behavior manager 175 may convey or pass the output 244 to the layout handler 180 via the event bus 185.

Referring now to FIG. 2D, depicted is a block diagram of a process 260 for modifying user interfaces in the system 100 for selecting configuration files. The process 260 may include or correspond to the operations of the application configuration service 105 and application 165 upon invocation of one of the routine logics 202 defined in the packages 228. Under the process 260, the layout handler 180 of the application 165 executing on the user device 110 may update, change, or otherwise modify the user interface elements 195 of the user interface 190 in accordance with the output 244. By setting the user interface 190, the layout handler 180 may associate or bind the states 204 (and the level 208) in the routine logic 202 of the package 228 to the user interface elements 195 of the user interface 190. In some embodiments, the layout handler 180 in conjunction with the behavior manager 175 may maintain the association or binding of the states 204 (and the level 208) of the routine logic 202 and the user interface elements 195 of the user interface 190. For example, the layout handler 180 may keep track of a relationship between the state 204 (and the level 208) of the most recently invoked routine logic 202 and the user interface elements 195 rendered or presented via the user interface 190.

In modifying, the layout handler 180 may determine whether to send a request 262 for content to the application configuration service 105 or to another remote service (e.g., associated with the developer of configuration file 155). In some embodiments, the layout handler 180 may be executed on the user device 110, for example, to identify content to be presented through the user interface 190. The output 244 may rely on at least one content item 264A-N (hereinafter generally referred to as content item 264). The content item 264 may be stored and maintained on a database (e.g., the database 115 as depicted), and may include images, videos, and other objects to be provided during the runtime of the application 165. If the output 244 does not specify the retrieval of the content item 264, the layout handler 180 may refrain from transmitting the request 262 for content to the application configuration service 105. The layout handler 180 may also continue to modify the user interface elements 195 of the user interface 190 in accordance with the output 244. On the other hand, if the output 244 specifies the retrieval of the content, the layout handler 180 may determine to send the request 262 for content to the application configuration service 105. The layout handler 180 may generate the request 262 for content to include at least one identifier referencing the content item 264 to be retrieved from the application configuration service 105. The identifiers may be specified by the output 244 from the now-current state 204 of the routine logic 202.

The content manager 145 executing on the application configuration service 105 may retrieve, identify, or otherwise receive the request 262 for content from the user device 110. In some embodiments, the content manager 145 may reside on a remote service separate from the application configuration service 105. The content manager 145 may parse the request 262 to identify the content item 264 to be provided to the user device 110 for presentation on the user interface 190. In some embodiments, the content manager 145 may use the identifier in the request 262 to access the database 115 to retrieve, fetch, or identify the content item 264 referenced by the identifier. The content item 264 may be information in a visual or audio medium, and may include an image, a video, an audio, or any other object to be presented on the user interface 190. For example, the content item 264 may include an audio to be played in conjunction with Exercise C for the endpoint associated with the invoked routine logic 202. With the identification, the content manager 145 may send, return, or otherwise provide the content item 264 to the user device 110.

The layout handler 180 may in turn retrieve, identify, or receive the content item 264 from the application configuration service 105 (or the remote service). Upon receipt, the layout handler 180 may insert, add, or otherwise include the content item 264 in the user interface 190. The layout handler 180 may include the content item 264 in one or more of the user interface elements 195 for presentation as specified in the output 244 from the routine logic 202. Concurrently, the layout handler 180 may modify the user interface elements 195 of the user interface 190 in accordance with the output 244. For example, the layout handler 180 may instantiate the user interface elements 195, set the color and other visual characteristics of the individual user interface elements 195 themselves, set the font and size of the text in the individual user interface elements 195, and assign the placement of the user interface elements 195 within the display of the user device 110.

In some embodiments, the layout handler 180 may generate or determine a render instruction using the output 244 specified by the routine logic 202. The output 244 may identify a set of instructions (e.g., in an original or lower-level format) corresponding to the respective user interface elements 195 to be included in the user interface 190. The render instructions may be in the form of a display list or render tree. Upon identification, the layout handler 180 may parse the instructions in the output 244 corresponding to the set of user interface elements 195. For each instruction, the layout handler 180 may generate an equivalent entry (e.g., a render tree node) to include in the render instructions. With the generation, the layout handler 180 may present the user interface elements 195 for the user interface 190 in accordance with the render instructions.

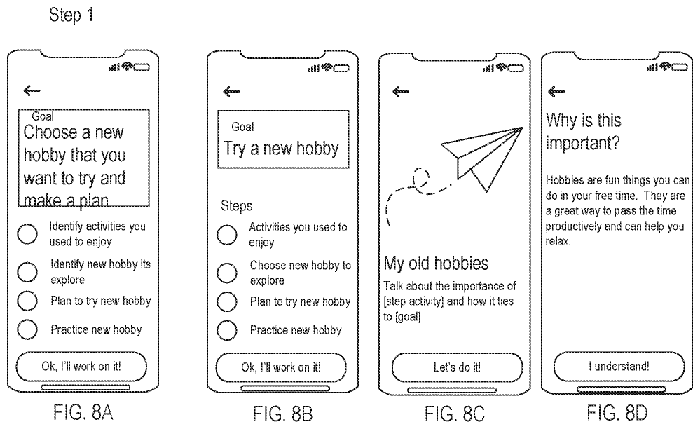

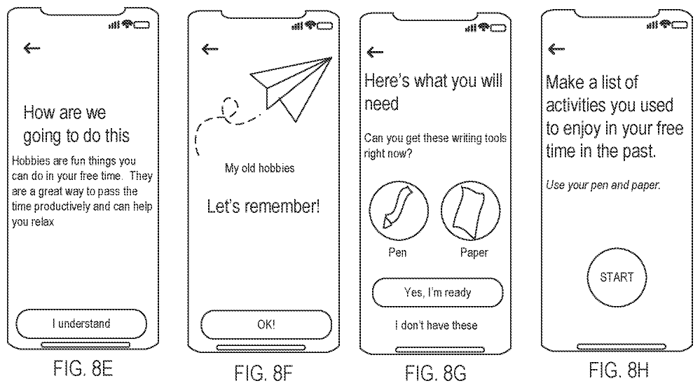

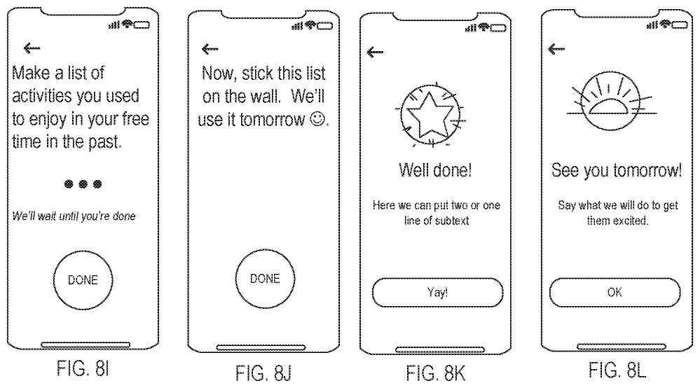

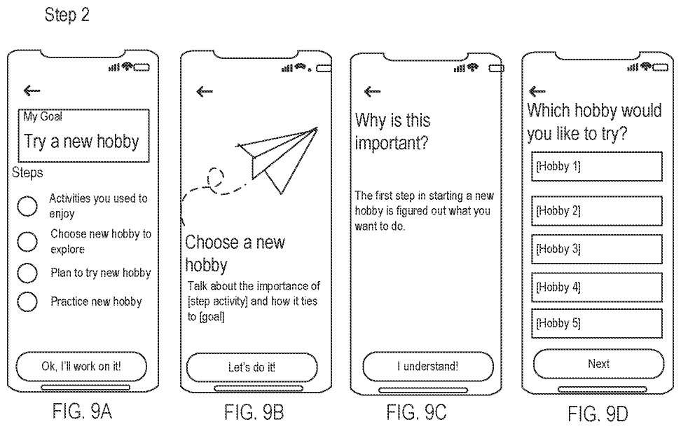

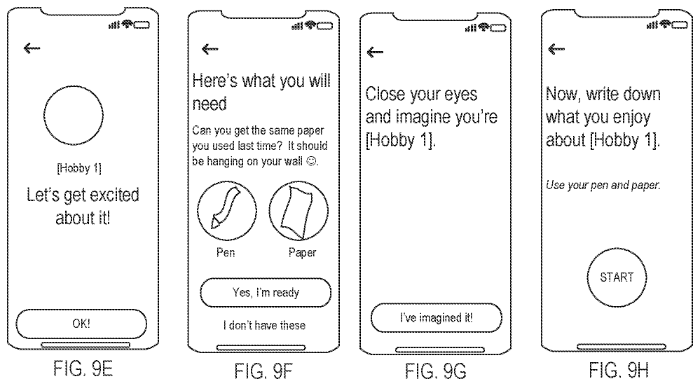

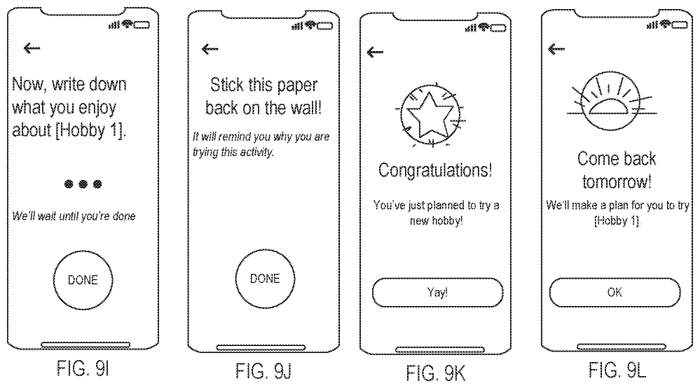

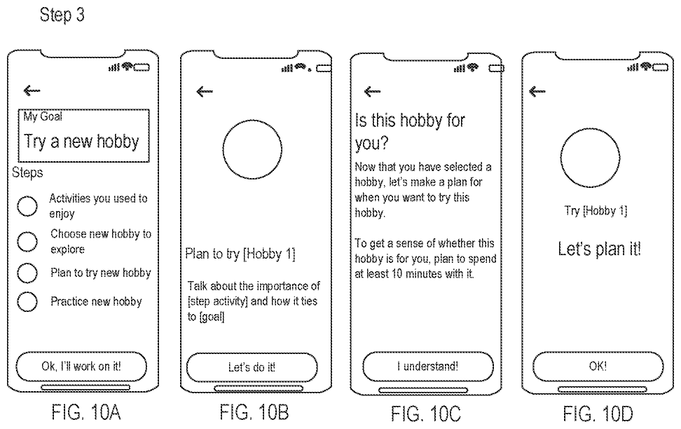

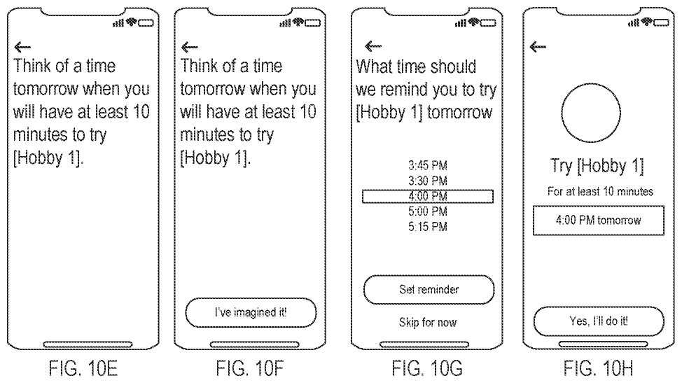

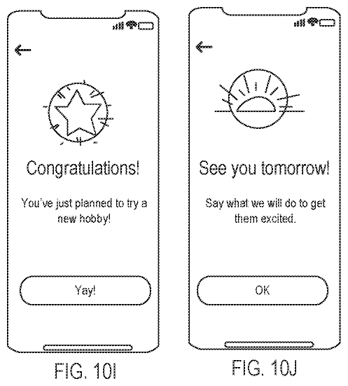

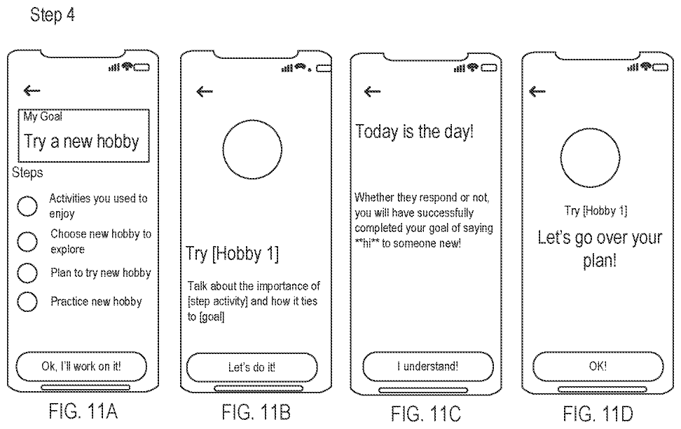

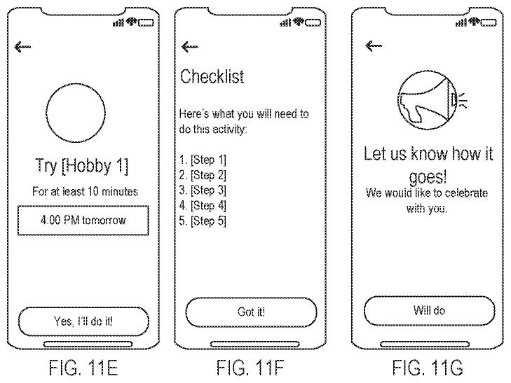

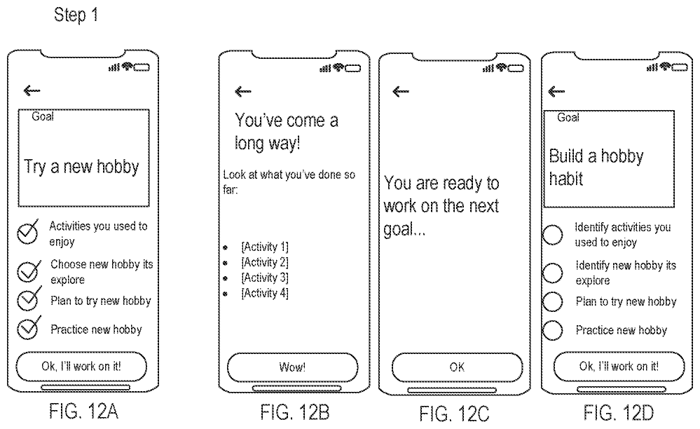

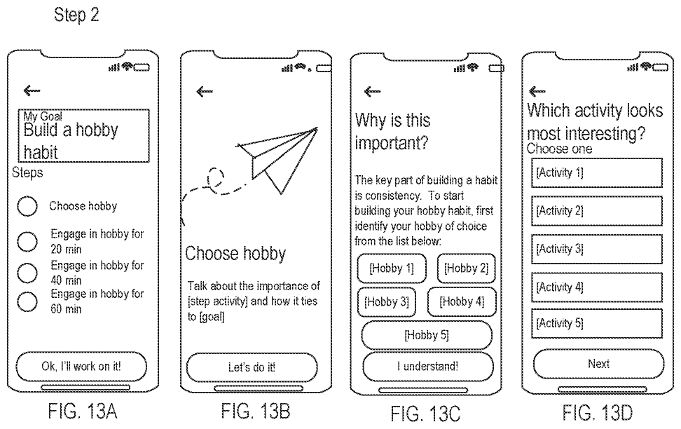

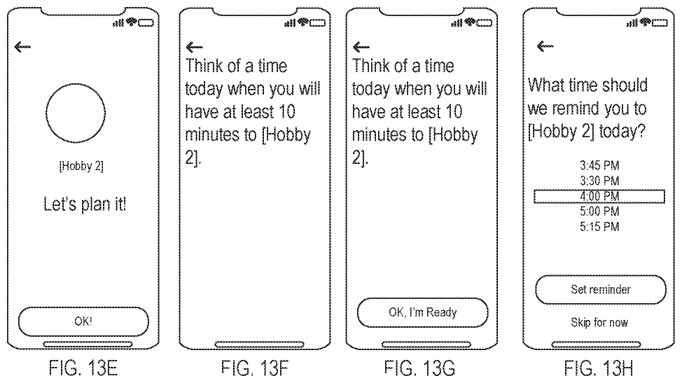

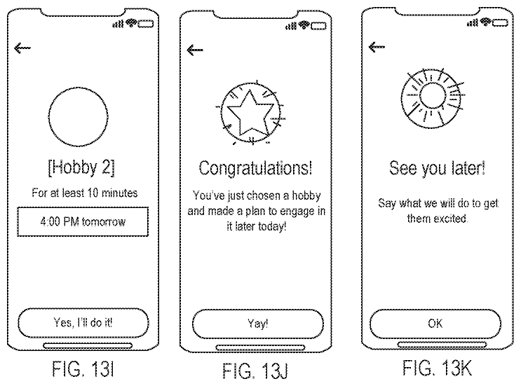

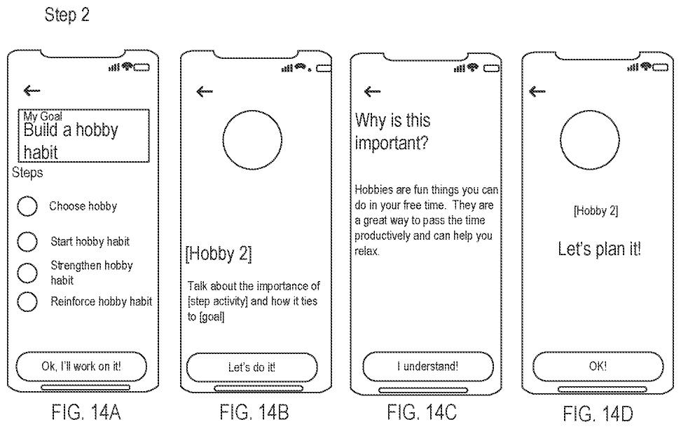

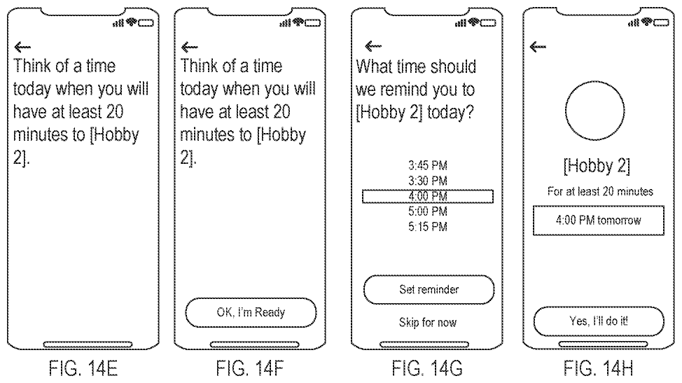

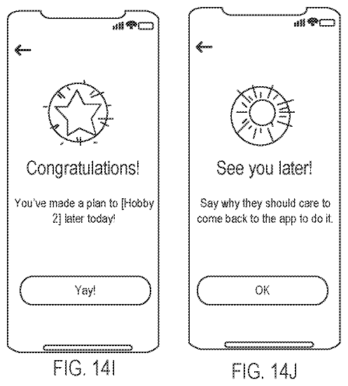

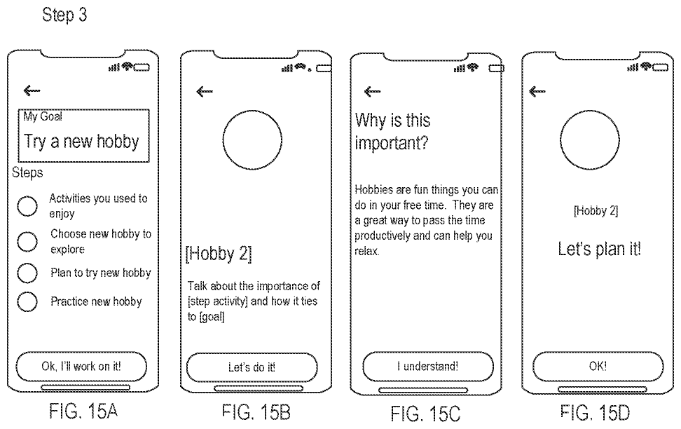

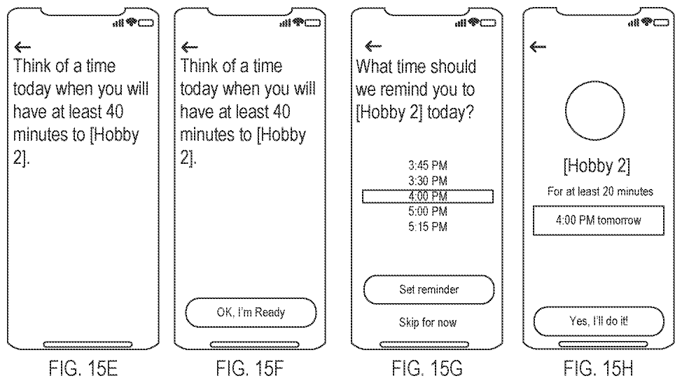

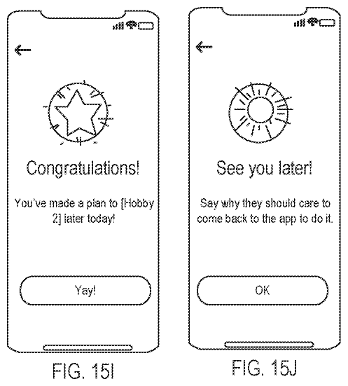

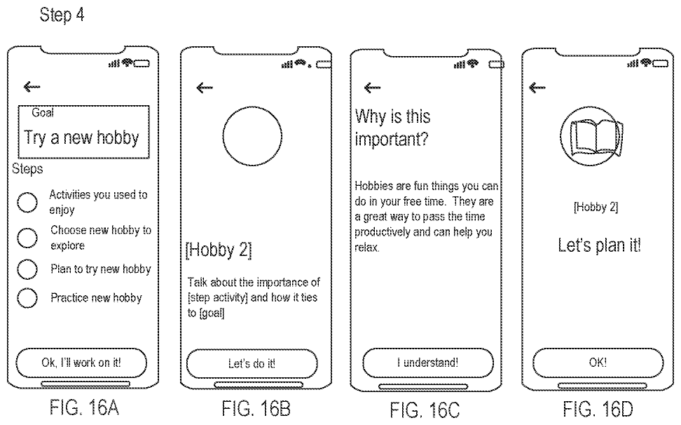

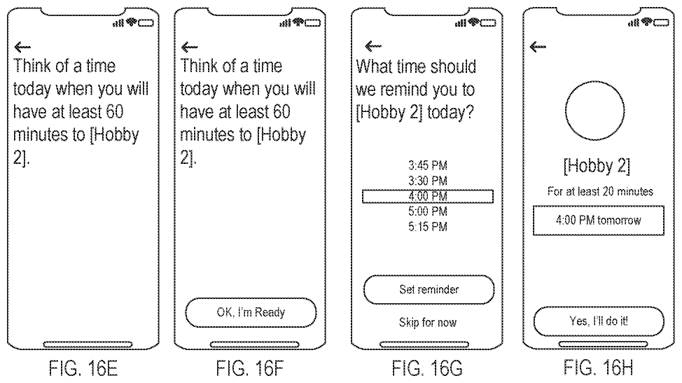

Examples of the content items 264 to be presented as user interface elements 195 on the user interface 190 are depicted in FIGS. 8A-L (level 1 for trying new hobbies, with selection of old hobbies), 9A-L (level 2 for trying new hobbies), 10A-J (level 3 for trying new hobbies), 11A-G (level 4 for trying new hobbies), 12A-D (level 1 for building a habit), 13A-K (level 2 for building a habit), 14A-J (level 2 for building a habit), 15A-J (level 3 for trying a new habit), and 16A-J (level 4 for a new habit). Similar to the questionnaire prompt 224, the content items 264 may also include prompts for questions to be responded to by the user 222. Each question depicted in the example may correspond to at least one content item 264 and may be associated with a transition 206 from one state 204 to another state 204. The user 222 may be presented with the depicted prompts upon interacting with the buttons or triggering the transitions 206 from one state 204 to another state 204.

Referring now to FIG. 2E, depicted is a block diagram of a process for assessing response data to provide configuration files in the system 100 for selecting configuration files. The process 280 may include or correspond to operations in the system 100 to evaluate responses and provide new packages. Under the process 280, the behavior manager 175 may send, transmit, or provide at least one response data 282 (sometimes referred to as a record entry) to the application configuration service 105. Upon detection of one or more events 242, the behavior manager 175 may write or generate the response data 282. The response data 282 may identify or include various events 242 in accordance with the set of activities as defined in the package 228 provided to the user device 110. For example, the information included in the response data 282 may include or identify the current state 204 and current level 208 of each routine logic 202; an update to the state 204 or the level 208 in the routine logic 202; an indication of completion or failure to complete the routine associated with a routine logic 202; the detected events 242, a timestamp at which each event 242 is detected, an identifier for the user 222, and an identifier for the user device 110, among others. With the generation, the behavior manager 175 may send the response data 282 to the application configuration service 105.

In some embodiments, the behavior manager 175 may call or invoke the profile creator 170 to aggregate, collect, or otherwise receive additional responses from the user 222 via the questionnaire prompt 224. The prompt 224 may be presented at a defined time, such as at the beginning of a day, an end of a day, once every 4-6 hours, once every week, or once a month, among others. For example, the user 222 may interact with a user interface element 195 to present the prompt 224 to present the set of questions. As described previously, the questions may ask the user 222 to indicate a desired endpoint to achieve, a condition to be addressed, preferences for performance of activities or routines, such as type of routine, frequency, duration, day of week, and time of day, among others. The questions may also be part of a clinical assessment interview as discussed above. The behavior manager 175 may receive the responses by the user 222 to the set of questions via the prompt 224 in a similar manner as discussed above. Upon receipt, the behavior manager 175 may include the responses from the user 222 into the response data 282.

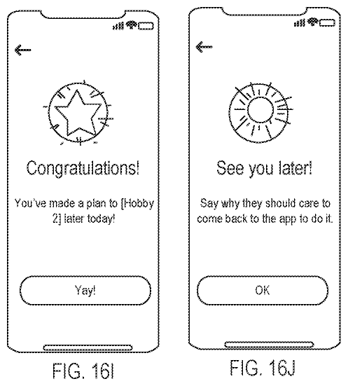

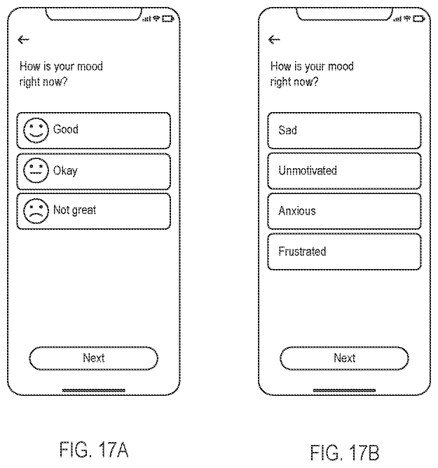

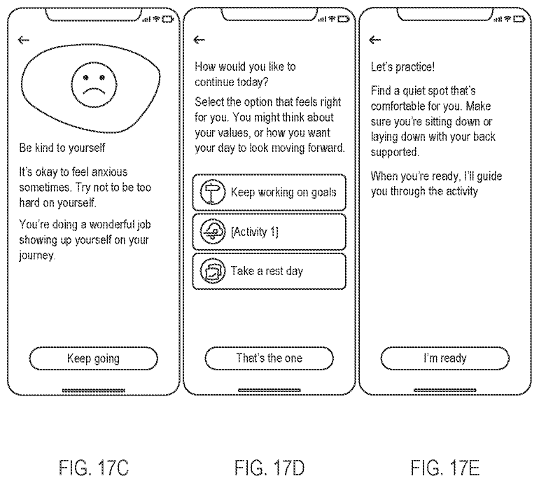

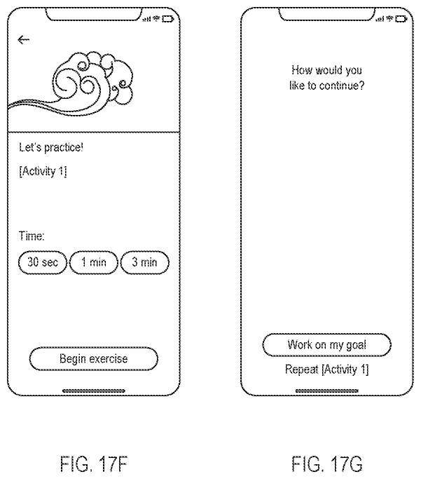

In some embodiments, the behavior manager 175 may determine or identify at least one state of the user 222 (e.g., a mood, emotional, behavioral, or physiological state) from the responses from the user 222 via the questionnaire prompt 224. Examples of the questionnaire prompt 224 for mood are depicted in FIGS. 17A-G. The mood check-in as depicted in the example may support the user 222 to increase likelihood of performing the activity (e.g., when prompted or at a later time). The check-in may also allow for the user 222 to build trust in performing the activities through the application 165 to meet the user 222 at their emotional state and work towards achieving the endpoint. The prompts 224 in the depicted example may allow the user 222 to indicate the user's mood (sometimes referred herein as emotional state), such as happiness, sadness, anger, fear, disgust, surprise, or excitement, among others. The response may be used to select configuration files 155 to provide targeted mitigations. Other states may include behavioral state (e.g., at rest, eating, working, studying, at leisure, interacting, playing, or exploring) or physiological state (e.g., resting, active, stressed, or intense), among others. The behavior manager 175 may invoke the profile creator 170 to present the questionnaire prompt 224 via the user interface elements 195 of the user interface 190 to prompt the user 222 to indicate the state. The questionnaire prompt 224 may be presented to the user 222 at a defined time (e.g., once every 4-6 hours, once a day in the evening, or once a week). Through the questionnaire prompt 224, the behavior manager 175 may receive the response indicating the state of the user 222. With receipt, the behavior manager 175 may include the responses from the user 222 in the response data 282.

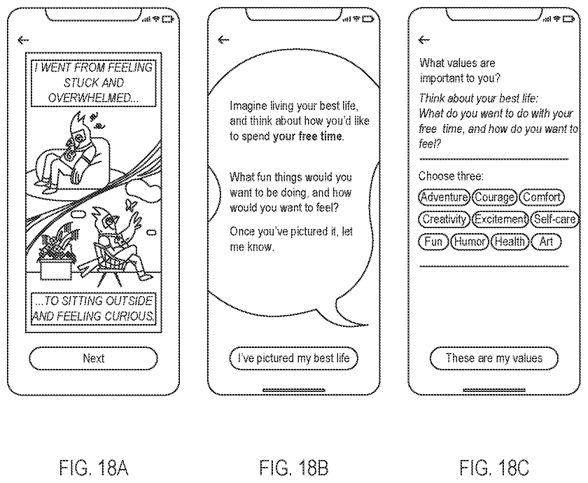

In some embodiments, the behavior manager 175 may determine or identify one or more personal values of the user 222 from the responses of the user 222 in response to the prompt 224. Examples of the questionnaire prompt 224 for personal values are depicted in FIGS. 18A-C. In the depicted examples, the interface in FIG. 18A may provide a scenario for the user 222 to consider, the interface in FIG. 18B may provide the user 222 an opportunity to explore values or biases for a goal or endpoint in mind, and the prompt in FIG. 18C may allow the user 222 to input one or more personal values to resurface throughout the session. The personal values may identify characteristics of activities that the user 222 desires to perform or endpoints that the user 222 identifies as objectives in performing the activities. The questionnaire prompt 224 may be presented to the user 222 at a defined time (e.g., check-in once every 4-6 hours, once a day in the evening, or once a week). Through the questionnaire prompt 224, the behavior manager 175 may receive the response indicating the one or more personal values. With receipt, the behavior manager 175 may include the responses from the user 222 into the response data 282.

In some embodiments, while running the routine logic 202 of the configuration file 155, the behavior manager 175 may determine, obtain, or otherwise identify a rating associated with the activity prior to performance. To identify, the behavior manager 175 may present the prompt 224 to indicate the rating associated with the activity prior to the performance of the activity. In addition, the behavior manager 175 may determine, obtain, or otherwise identify a rating associated with the activity subsequent to the performance via the application 165. To identify, the behavior manager 175 may present the prompt 224 to indicate the rating associated with the activity subsequent to the performance of the activity. The ratings may be obtained from the responses of the user 222 in response to one or more prompts 224 for indicating the ratings. The rating prior to performance may indicate a self-assessed value for expectation by the user 222 at addressing the condition or toward achieving the endpoint by performing the activity identified in the prompt 224. The rating subsequent to performance may identify a self-assessed value for an experience by the user 222 of addressing the condition or toward achieving the endpoint subsequent to performing the activity identified in the prompt 224.

Regarding the ratings, the application 165 through the configuration file 155 may provide lesson content explaining the connection between thoughts, emotions, and behavior to inform the user 222 about the premise of cognitive restructuring and how the activities address the condition. With the configuration files 155, the application 165 may provide one or more interactive activities to help the user 222 understand thought patterns that contribute to defeatist beliefs associated with negative symptoms.

For example, in an orientation phase, after the user 222 is introduced to the pre- and post-activity survey concept, the behavior manager 175 surveys the user a given number of times (e.g., 4 times). Before the user 222 starts an activity, the behavior manager 175 may prompt the user 222 with an anticipatory question regarding their activity to select from a scale of 1 to 10. After the user 222 completes an activity, the behavior manager 175 may prompt the user 222 a reflection question regarding their activity to select from a scale of 1 to 10. In the active phase, the behavior manager 175 may prompt the user 222 before and after each activity with the survey. Before the user 222 starts an activity, the behavior manager 175 may prompt the user 222 with an anticipatory question regarding their activity to select from a scale of 1 to 10. After the user 222 completes an activity, the behavior manager 175 may prompt the user 222 with a reflection question regarding their activity to select from a scale of 1 to 10.

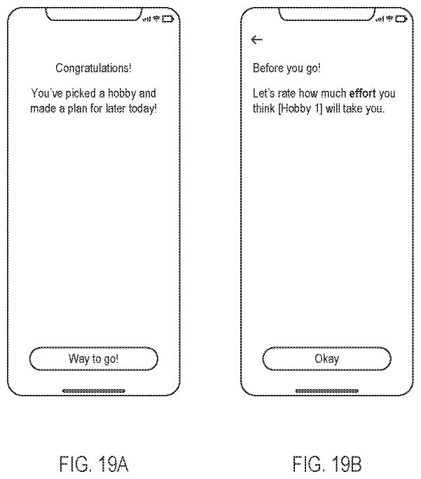

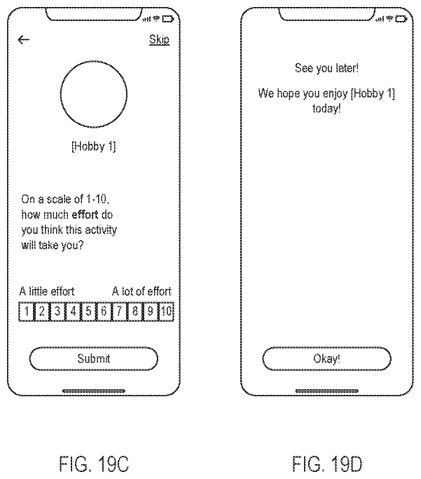

Examples of the questionnaire prompt 224 for self-assessed ratings for pre- and post-activity are depicted in FIGS. 19A-D. In the depicted example, the prompts may encourage users to practice noticing their expectations with each activity. The question in the prompt 224 may be a pre/post-activity question and the response data may be used to reflect back a user's own indications to combat defeatist beliefs and show growth. These self-assessments by the user 222 may be used to reflect shifts in perception. Showing improvements may be a powerful driver of trust, leading to a change in user perception that may motivate users 222 to more closely adhere to the digital therapeutic provided through the configuration files 155. After performing the activity, the user 222 may be prompted to reflect on the experience from performing the activity. The user 222 may be shown pre- and post-activity responses to encourage adherence and continued performance of the activities presented via the application 165. Through the questionnaire prompt 224, the behavior manager 175 may receive the response indicating the ratings. With receipt, the behavior manager 175 may include the responses from the user 222 into the response data 282.

The progress tracker 150 executing on the application configuration service 105 may change, modify, or otherwise update the user profile 160 maintained on the database 115, using the response data 282. As discussed previously, the user profile 160 maintained on the database 115 may be used to keep track of the progress of the user 222 with each routine provided via the package 228. The progress tracker 150 may retrieve, identify, or otherwise receive the response data 282 from the user device 110. Upon receipt, the progress tracker 150 may parse the response data 282 to extract or identify the information included therein. In some embodiments, the progress tracker 150 may store and maintain the response data 282 (e.g., including the indication of the state of the user 222, one or more personal values, or ratings) on the database 115. The progress tracker 150 may use a log record associated with the user profile 160 or the user 222 to store the response data 282 on the database 115. The log record may be a data structure associated with the user profile 160.

Based on the information parsed from the response data 282, the progress tracker 150 may set, update, or otherwise modify the user profile 160. Using the identifier for the user 222 from the response data 282, the progress tracker 150 may identify the user profile 160 associated with the user 222. From the user profile 160, the progress tracker 150 may identify a currently recorded level 208 of the user 222. The level 208 may correspond to a stage or a progression towards achieving a given endpoint, a set of activities, or addressing of the condition, among others. For each configuration file 155 selected for the user 222, the user profile 160 may identify the current state 204 and the current level 208 in the routine logic 202.

With the identification, the progress tracker 150 may determine whether there is a transition from the current level 208 to a next level 208 based on the response data 282. From the response data 282, the progress tracker 150 may extract or identify the level 208 of the user 222. In some embodiments, the progress tracker 150 may determine whether to transition the level 208 for the user 222 towards achieving the endpoint. When the user 222 has accomplished the endpoint or the activity, the level 208 indicated in the response data 282 may be higher than the level 208 currently identified in the user profile 160. When the user 222 has not accomplished the endpoint or the activity, the level 208 indicated in the response data 282 may be the same or lower than the level 208 currently identified in the user profile 160. In some embodiments, the progress tracker 150 may determine the new state 204 and the level 208 in accordance with the routine logic 202 of the configuration file 155 selected for the user 222 using the one or more interactions as identified in the response data 282. The new level 208 may be closer to achieving a different endpoint than the previous level 208 as indicated in the user profile 160.

The progress tracker 150 may compare the level 208 from the user profile 160 with the level 208 identified in the response data 282 to determine whether there is a transition. If the levels are not different, the progress tracker 150 may determine that there is no transition from the current level 208 for the user 222. If the levels are different, the progress tracker 150 may determine that there is a transition between the current level 208 to the next level 208. The progress tracker 150 may set the state 204 and the level 208 in the user profile 160 to the state 204 and the level 208 respectively as identified in the response data 282. In some embodiments, the progress tracker 150 may determine or identify the transition to a higher or lower level based on the identified levels 208. When the current level 208 is lower than the level 208 identified from the response data 282, the progress tracker 150 may determine the transition as to a higher level. Conversely, when the current level 208 is higher than the indicated level 208, the progress tracker 150 may determine the transition as to a lower level.

In addition, the progress tracker 150 may set, update, or otherwise modify the parameters 226 in the user profile 160 using the information from the response data 282. As discussed previously, the parameters 226 may identify the endpoint to be achieved and the condition of the user 222 to be addressed, the state of the user 222, a type of routine, a frequency, duration, and time (e.g., day of week or time of day) for the routine, among others. In some embodiments, the progress tracker 150 may adjust, set, or change the frequency and duration based on the indication of success or failure of the routine selected for the user 222 or update in the state 204 or the level 208. For example, the progress tracker 150 may increase the frequency or duration for the routine when the response data 282 indicates successful completion of the routine. Conversely, the progress tracker 150 may decrease the frequency or duration for the routine when the response data 282 indicates failure to complete the routine.

In some embodiments, the progress tracker 150 may identify or determine at least one progression metric with respect to the personal values based on the response data 282. The response data 282 may identify the performance of the activity towards achieving the endpoint, such as the one or more interactions with the content items presented via the user interface 190 of the application 165. The progression metric may identify or correspond to a measure of improvement or degradation with respect to satisfying the personal values from performing the activities specified by the configuration file 155. For instance, for a personal value of excitement, the progress tracker 150 may determine a relatively higher progression metric when the response data 228 indicates that the user is satisfied with the activities performed. With the determination, the progress tracker 150 may transmit, send, or otherwise provide a connection between the progression metrics and the personal values for presentation via the user interface 190 of the application 165. For example, the connection may be presented as part of the prompt 224 or on the user interface 190 subsequent to the presentation of the prompt 224. In some embodiments, the progress tracker 150 may provide the progression metrics as part of the subsequent package to be provided to the application 165. The progress metrics may be presented to the user 222 to encourage reflection and drive perception of progress.

In some embodiments, the progress tracker 150 may compare the ratings obtained prior to the performance of the activity and subsequent to the performance of the activity as identified in the response data 282. Based on the comparison, the progress tracker 150 may calculate, generate, or otherwise determine a metric identifying a difference between the two ratings. With the determination, the progress tracker 150 may transmit, send, or otherwise provide a comparison (or the difference metric, or both) between the ratings for presentation via the application 165. For example, the comparison may be presented as part of the prompt 224 or on the user interface 190 subsequent to the presentation of the prompt 224. The presentation of the comparison may reinforce the concept of experimentation in performing the activity regardless of the anticipated outcome for the user 222. The presentation of the pre- and post-activity assessments may also challenge preconceptions and defeatist views to the user 222 with evidence of the user's own experiences that progressed differently from expectation. In some embodiments, the progress tracker 150 may provide the comparison or the difference metric as part of the subsequent package to be provided to the application 165.

With the updating of the user profile 160, the progress tracker 150 may modify, set, or change the endpoint to be achieved. In some embodiments, the progress tracker 150 may modify, set, or change the condition of the user 222 based on the information parsed from the response data 282. The information may include the responses received via the prompt 224. The progress tracker 150 may replace, change, or otherwise set the endpoint or the condition of the user 222 indicated in the user profile 160 with the endpoint or the condition respectively as indicated in the response data 282. The changing of the endpoint and the condition may result in the changing of the parameters 226 in the user profile 160. In some embodiments, the progress tracker 150 may calculate, determine, or generate new measures based on the updated parameters 226. The generation of the measures using the parameters 226 may be performed in a similar manner as discussed above. For example, the progress tracker 150 may use a function to calculate values for the characteristic of the user 222 and the likelihood that the user 222 will perform a given routine, among others. Upon generation, the progress tracker 150 may store and maintain the new measures with the user profile 160 on the database 115.

The configuration selector 135 may select, identify, or otherwise determine one or more new endpoints for the user 222. The identification of the endpoints (and activities associated with the endpoint) may be in a similar manner as discussed above and may be based on the parameters 226 of the user profile 160 and the selection criterion 210. For example, the change to the user profile 160 may include the update in the state 204 or the level 208 or the indication of successful completion or the increase in the duration or the frequency for the routine. In this case, the configuration selector 135 may select a next endpoint, with activities of an increased duration and frequency. In contrast, the change to the user profile 160 may include the update in the state 204 or the level 208 or the indication of a failure to complete or decrease in the duration or the frequency for the routine. In this scenario, the configuration selector 135 may select the routine with a decreased duration and frequency. Continuing on, the change to the user profile 160 may include a modification in the endpoint or the condition of the user 222. Based on this change, the configuration selector 135 may select activities for the new endpoint or condition.

In some embodiments, the configuration selector 135 may select the new endpoints (or activities) for the user 222 using information derived from the response data 282 received via the prompt 224. The information may include those generated by the progress tracker 150 from the response data 282. In some embodiments, the configuration selector 135 may identify or select the new endpoint based on the state of the user 222 (e.g., mood, emotional, behavioral, or physiological state) as indicated in the response data 282. For example, when the state of the user 222 indicates a sadness state, the configuration selector 135 may select an endpoint with the aim of soothing the user 222 while performing activities to address the condition of the user 222. In some embodiments, the configuration selector 135 may identify or select the new endpoint based on the personal values as identified by the user 222. For instance, the configuration selector 135 may select an endpoint aimed at providing activities related to adventure, self-care, or art as indicated in the response data 282.

In some embodiments, the configuration selector 135 may identify or determine the new endpoints for the user 222 from at least one of the social domain, the recreation domain, or the productivity domain to address the condition. Based on the information derived from the response data 282, the configuration selector 135 may modify, update, or otherwise change the domain to the new domain for the user 222. For example, when there is a completion of the last level 208 for a given domain (e.g., social domain) indicating that the user has improved along the domain, the configuration selector 135 may select a different endpoint (e.g., recreation or productivity domain). In this manner, the configuration selector 135 may adaptively and dynamically select the endpoints for the user 222 across varying domains to address the user's condition based on the response data 282 from the user 222. This may allow the user 222 to perform activities specified by the endpoint and build skills along the identified domain.

With the determination of the new endpoints, the configuration selector 135 may select one or more configuration files 155 from the overall set of configuration files 155 to provide to the application 165. The selection of the configuration files 155 may be in a similar manner as discussed previously, using the newly selected endpoint or activities. In some embodiments, the configuration selector 135 may select one or more configuration files 155 based on the transition in levels 208. Using the selected configuration files 155, the configuration packager 140 may generate at least one new package 228′. The package 228′ may be generated in a similar fashion as detailed above and may include instructions for configuring functionalities to be performed on the application 165 in accordance with the routine logic 202 as defined by the configuration file 155. Upon generation, the configuration packager 140 may provide the package 228′ with the newly selected configuration file 155 to the application 165. In turn, the application 165 may receive and load the package 228′. Using the package 228′, the application 165 may repeat the operations as described above.

By selecting and providing configuration files 155 in this manner, the application configuration service 105 may configure the functionalities of the application 165 customized to the responses indicated by the user 222. The configuration files 155 may provide a greater range of experiences to the user 222 and a sequence of content via the content items 264 adapted to the state and interactions from the user 222 in accordance with the routine logic 202. The configuration files 155 thus may improve the quality of human-computer interaction (HCl) between the user 222 and the application 165. In the context of digital therapeutics, the configuration file 155 and the content items 264 identified therein may lead to higher user engagement with the application 165. With higher likelihood of interactivity, the digital therapeutic provided by the application 165 may have higher efficacy in addressing the condition and increased adherence of the user 222 with the digital therapeutic. The configuration files 155 may also decrease the consumption of computing resources (e.g., on both the user device 110 and the application configuration service 105) that would have otherwise been used to provide and load irrelevant content on the application 165. Furthermore, the configuration files 155 may also reduce resorting to having to update the application 165 itself to provide additional functionality, further saving computing resources. The configuration files 155 (and by extension the packages 228) may reduce the expenditure of network bandwidth from back-and-forth communications associated with requesting and retrieving the content.

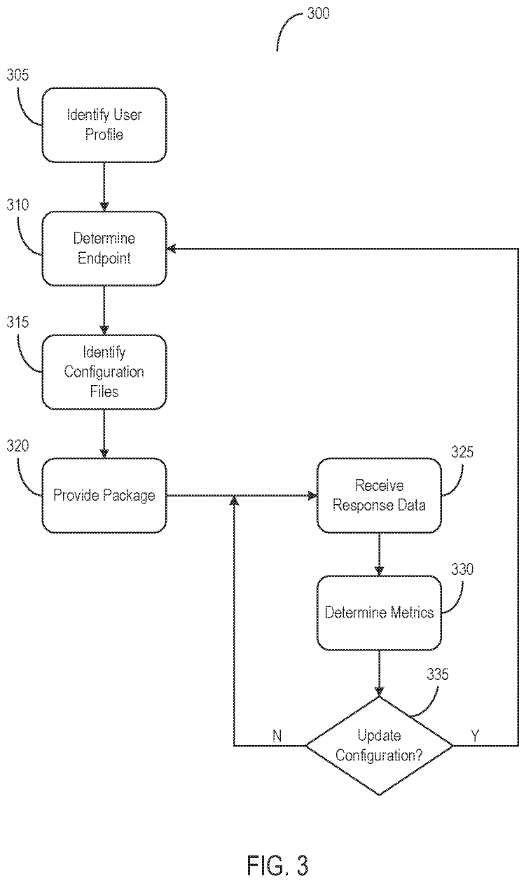

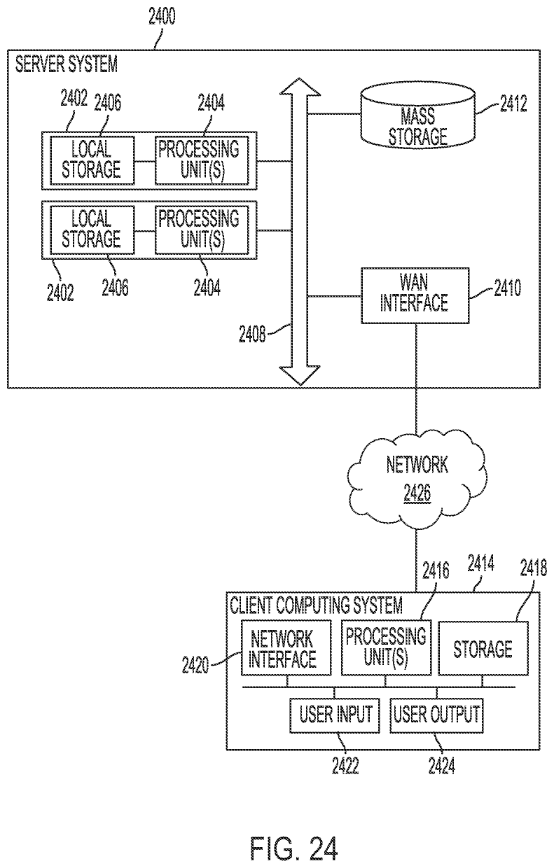

Referring now to FIG. 3, depicted is a flow diagram of a method 300 of selecting configuration files for applications. The functionalities of method 300 may be implemented by using or performed by any of the components discussed herein in conjunction with FIGS. 1-2E, such as the application configuration service 105 and the user device 110, or FIG. 24, such as the computing system 2400. In overview, a server may identify a user profile (305). The server may determine an endpoint for the user (310). The server may identify configuration files (315). The server may provide a package (320). The server may receive response data (325). The server may determine metrics (330). The server may determine whether to update the configuration (335). If the determination is to update, the server may select new endpoints and repeat the functionalities from (310). Otherwise, if the determination is not to update, the server may wait for additional response data and repeat the functionalities from (325).

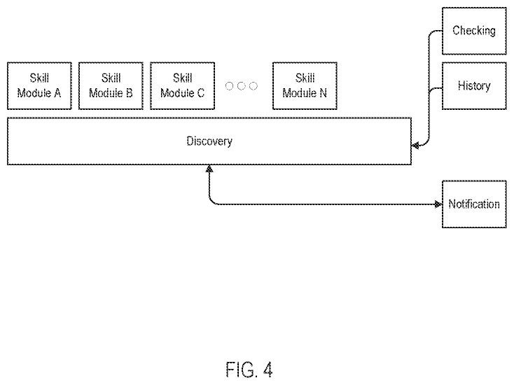

Referring now to FIG. 4, depicted is a block diagram of an architecture for a system for adaptive goal setting for selecting configuration files. The architecture may be implemented using the components of the system 100, such as the application configuration service 105 and the application 165 on the user device 110. As depicted, the architecture may partition the adaptive goal setting (AGS) into three parts. First, the discovery component may select a skill (e.g., the endpoint) to be provided to the user. The selection may be based in part on user history. Second, the skills may be organized in corresponding modules A, B, C, . . . . N (e.g., in the form of the configuration files 155) for the user. All the logic for the skill may be included into the module, which may be self-contained, such that each time the module runs, the behavior of the module may be new and unique. Third, the notification component may be used to remind and encourage the user to perform the skills as organized in the modules. Subsequently, the discovery component may perform a check on the user to determine whether the user has completed the skill modules. Based on the determination, the discovery component may select new skill modules, and the functionalities of the architecture may be repeated again.

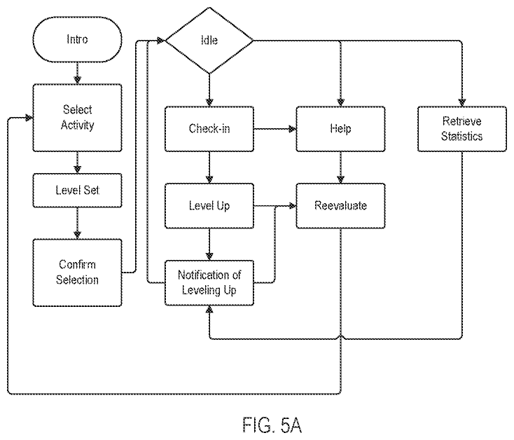

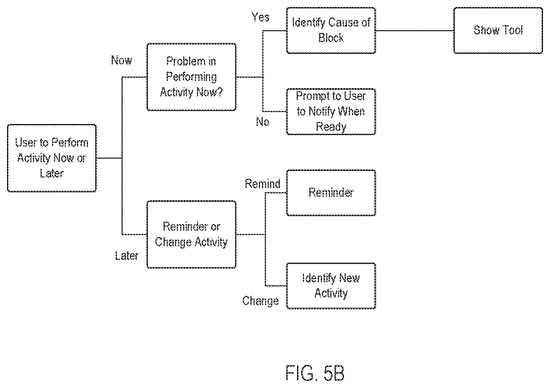

Referring now to FIG. 5A, depicted is a flow diagram of a method of performing adaptive goal setting in selecting configuration files. The method may be performed or implemented using the components of the system 100, such as the application configuration service 105 and the user device 110. For example, at least one configuration file 155 may be used to define and carry out at least a portion of the depicted method. As illustrated, a system may present an introduction to a user. From the introduction, the system may receive a selection of activities by the user. The system may set a level for the activities of the user. The user may confirm the selection of the level and the activities via interaction. The system may monitor user interactions to determine whether the user is idle. When idle, the system may check-in with the user to prompt the user to perform the activity. Upon completion, the system may update a level of the user, proceed to notify the user of the update to the change in the level, and perform a re-evaluation of the user. The system may also present a help prompt to instruct the user how to perform the activity. Depending on the results, the system may further perform a re-evaluation. In the meanwhile, the system may also retrieve statistics on the user with respect to the activities.

Referring now to FIG. 5B, depicted is a flow diagram of a method of performing activities in accordance with the configuration files. The method may be performed or implemented using the components of the system 100, such as the application configuration service 105 and the user device 110. For example, at least one configuration file 155 may be used to define and carry out at least a portion of the depicted method. As illustrated, the system may determine whether the user is to perform an activity now (e.g., within a time window from the current time) or later (e.g., outside the time window from the current time). If the activity is to be performed now, the system may determine whether there is a problem in performing the activity now. If yes, the system may identify a cause of the blockage or hindrance and may show a tool (e.g., a user interface) to address the issue. On the other hand, if it is determined that the activity is to be performed at a later point, the system may determine whether to provide a reminder or to change the activity. When the determination is to remind, the system may present a reminder. When the determination is to change, the system may identify a new activity.

### B. Methods of Ameliorating Experiential Negative Symptoms of Schizophrenia in Subjects in Need Thereof

An individual suffering from experiential negative symptoms of schizophrenia may be afflicted with a decrease or reduction in certain functions and abilities that can affect the individual's quality of life and daily routines. The severity or the impact of the negative symptoms of schizophrenia on the individual can be measured using various scales, such as a Clinical Assessment Interview for Negative Symptoms Motivation and Pleasure (CAINS-MAP) scale, among others. The CAINS-MAP scale may measure the individual on various domains related to the experiential negative symptoms of schizophrenia, such as recreational, social, or productivity activities, among others.

A digital therapeutic application configured with an adaptive goals setting (AGS) framework may be provided to such an individual to improve the experiential negative symptoms. Under the AGS framework, the application may adaptively determine endpoints based on the user response and feedback. Each endpoint may define a set of one or more activities to be performed by the user through the application towards addressing the experiential negative symptoms of schizophrenia. The endpoints may be determined from a set of domains related to the components of the measurement scale (e.g., social, recreational, and productivity) specific to schizophrenia, and each of these domains may specify activities to be performed pursuant to the domain.

Using the determined endpoint, the application may provide selected configuration files to be loaded to present content items prompting the user with the specified activities. For example, the application can identify an endpoint in the social domain specifying activities such as conversing with a clinician in person, and then be provided with a corresponding configuration file to provide. The configuration file may be converted from a human-readable instruction format to a format for reading and execution by the application. Upon loading, the application can present content items as identified by the configuration file through a user interface prompting the user to perform a social interaction activity. The application may then monitor for interactions by the user with the user interface elements indicating completion of the specified activities.

With additional user response and feedback data, the application may update the endpoint for the user, and may be provided other configuration files selected according to the new endpoint. Continuing with the previous example, when the endpoint in the social domain associated with the social interaction activity is determined to be completed, the application may determine a new endpoint in a productivity domain that specifies that the user is to complete a physical activity. Upon loading of the corresponding configuration file, the application may present content items specified by the file prompting the user to perform the activity. This may be repeated a number of time instances over a time period to select and provide configuration files with different activities for the user to perform. By adaptively and dynamically determining the endpoints for the user across varying domains to address the experiential negative symptoms of schizophrenia using response data, the application may provide selected configuration files with content items that activities targeted at improving the user along the particular domain. Through repetitive uses of the application over time, the user of the application may experience an improvement in the experiential negative symptoms of schizophrenia as measured using various scales, as documented herein.

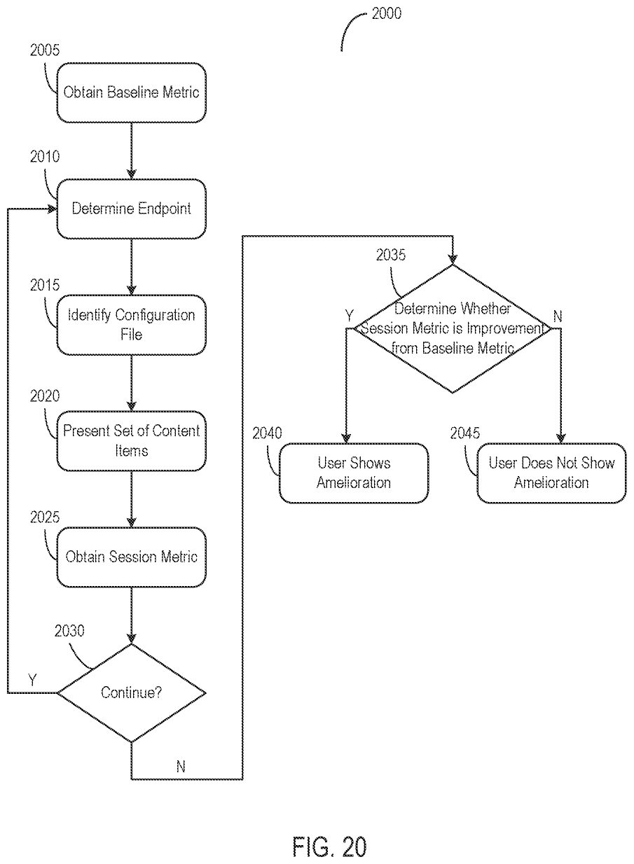

Referring now to FIG. 20, depicted of a flow diagram of a method 2000 of ameliorating experiential negative symptoms of schizophrenia in a user in need thereof. The method 2000 may be performed by any components or actors described herein, such as the application configuration service 105, the user device 110, or the user 222, among others. The method 2000 may be used in conjunction with any of the functionalities or actions described herein in Section A or in Examples 1 and 2 in Section B. In brief overview, the method 2000 may include obtaining a baseline metric (2005). The method 2000 may include determining an endpoint (2010). The method 2000 may include identifying a configuration file (2015). The method 2000 may include presenting the set of content items (2020). The method 2000 may include obtaining a session metric (2025). The method may include determining whether to continue (2030). The method 2000 may include identifying or determining whether the session metric is an improvement over the baseline metric (2035). In some embodiments (e.g., as depicted), the method 2000 may include determining that the user shows amelioration, when the session metric is an improvement over the baseline metric (2040). The method 2000 may include determining that the user does not show amelioration, when the session metric is not an improvement over the baseline metric (2045).

In further detail, the method 2000 may include retrieving, identifying, or otherwise obtaining a baseline metric (2005). The baseline metric may be associated with a user (e.g., the user 222) diagnosed with schizophrenia with negative symptoms, prior to performing any activity via a digital therapeutics application (e.g., the application 165 or the Study App described herein). The experiential negative symptoms of schizophrenia of the user may include, for example, one or more of a blunted affect, alogia (reduction in quantity of words spoken), avolition (reduced goal-directed activity due to decreased motivation), asociality, and anhedonia (reduced experience of pleasure), among others.

The user may be of any demographic or trait, such age (e.g., an adult (above age of 18), late adolescent (between ages of 18-24)) or gender (e.g., male, female, or non-binary). The user may also be receiving a stable dose of an antipsychotic medication for at least a period of time (e.g., 12 weeks) prior to the first activity through the digital therapeutics application. The antipsychotic medication may include, for example, risperidone, quetiapine, olanzapine, ziprasidone, paliperidone, or aripiprazole, among others. Other medications may be used such as iclepertin GlyT1 inhibitor.

The baseline metric may be obtained (e.g., by a clinician or the application 165) prior to the user performing an activity through a digital therapeutics application (e.g., the application 165 or the Study App described herein). In some embodiments, the baseline metric may be obtained from a source separate from the application. For instance, a clinician examining the user may ascertain the basement metric, and provide the baseline metric for storage. In some embodiments, a computing system (e.g., the application 165 or the application configuration service 105) may determine the baseline metric via the user interactions with a prompt (e.g., a questionnaire for the metric) presented through the user interface. In some embodiments, the baseline metric may be obtained in advance of a period of time (e.g., 1-20 weeks) before the first activity. The baseline metric may include, for example, a score for one or more of the following: a Clinical Assessment Interview for Negative Symptoms (CAINS) Motivation and Pleasure Scale, Clinical Assessment Interview for Negative Symptoms, Expressivity Scale (CAINS-EXP), Positive and Negative Syndrome Scale (PANSS), Personal and Social Performance Scale (PSP), a Defeatist Beliefs Subscale of the Dysfunctional Attitudes Scale (DAS), Patient Global Impression of Improvement Scale (PGI-I), Patient Global Impression of Severity Scale (PGI-S), Clinical Global Impression of Severity Scale (CGI-S), WHO Disability Assessment Schedule 2.0 (WHODAS 2.0), or Schizophrenia Quality of Life Scale Revision 4 (SQLS-R4), among others.

The user that is to perform activities through the digital therapeutics application may have a baseline metric below a certain threshold. The threshold may indicate that the user is experiencing or suffering from a moderate to severe symptom severity in connection with the negative symptoms of schizophrenia. The user may have experienced at least moderate to severe negative symptom severity (e.g., as measured by the baseline metric) prior to the first activity. For example, the user may have a score of ≤30 on the Motivation and Pleasure Scale (MAPS) prior to the first activity.

The method 2000 may include identifying or determining at least one endpoint for ameliorating the experiential negative symptom (2010). A computer system (e.g., the application configuration service 105 or the user device 110) may identify or determine the endpoint from a set of endpoints to ameliorate the experiential negative symptom. The endpoint may correspond to a mark of completion of a defined set of activities aimed at ameliorating the experiential negative symptom. In some embodiments, the endpoint (e.g., prior to any activity by the user) may be selected from a set of endpoints based on a baseline assessment of the user for the experiential negative symptoms of schizophrenia. In some embodiments, the endpoint (e.g., subsequent to at least one activity performed by the user when prompted) may be selected from the set of endpoints based on the response data identifying the one or more interactions by the user with the set of content items presented via an application from a previous time instance.

In some embodiments, the set of endpoints may be associated with one or more classifications or domains, such as a social domain, a recreation domain, or a productivity domain. The domains for the set of endpoints may be associated with the type of metric for which the user is evaluated. For instance, the social, recreation, and productivity domains may be related to the CAINS-MAP scale, which may be used to measure various aspects or domains of the experiential negative symptoms of schizophrenia in the user. The endpoints associated with the social domain may identify activities to improve sociality of the user, the endpoints associated with the recreation domain may identify activities for performing leisurely exercises or tasks for the user, and the endpoints associated with the productivity domain may include activities for improving attention or work habits of the user. Each domain may be associated with a number of aspirations. For example, the recreation module may have the aspirations of hobbies, creativity, and exercise. The productivity module may have washing dishes as an example of an activity. The endpoint may also be associated with a number of levels.

The computing system may select the endpoints based on any number of factors (e.g., as described in Section A). In some embodiments, the selection of the endpoint may be based on the baseline assessment for the first activity. The selection may be based on subsequently obtained assessments for subsequent activities. In some embodiments, the selection of the endpoint for subsequent activities may be based on response data identifying interactions with the previously presented content items. In some embodiments, the selection of the endpoint (for the first or subsequent activities) may be based on one or more personal values indicated by the user. The personal values may identify characteristics of activities that the user desires to perform or endpoints that the user identifies as objectives in performing the activities.

In some embodiments, the selection of the endpoint (for the first or subsequent activities) may be based on a state indicated by the user. The state may be associated with a mood, a behavior, or a physiological state of the user. In some embodiments, the computing system may identify or select the endpoint based on a transition from one level to another level as determined using the response data after performing at least one activity. In some embodiments, the computing system may determine an endpoint to change from one domain to another domain. For example, when evaluating the user on the CAINS-MAP scale, the computing system may initially identify an endpoint associated with the productivity domain for the user. With additional response data, the computing system may update the endpoint to an endpoint associated with the social or recreational domain. For instance, when the response data indicates a statistically significant improvement in the productivity domain (e.g., as shown by the metrics) or the determination is made to transition to another level, the computing system may change the endpoint for the user.

The method 2000 may include identifying or selecting a configuration file from a plurality of configuration files based on the endpoint (2015). Based on the endpoint, the computing system may identify or select the configuration file (e.g., the configuration file 155) corresponding to the endpoint. The configuration file may identify a set of content items prompting the user to perform one or more activities towards achieving the determined endpoint for ameliorating the experiential negative symptom. In some embodiments, at least one of the configuration files may identify a criterion defining a measure to select another of the plurality of configuration files. The measure may identify, for example, a likelihood that the user is to perform the activity or a predicted efficacy of the activity on the user towards achieving the endpoint, among others. In some embodiments, the configuration file may be selected based on a change in the endpoint determined using the response data from a previous time instance.

In some embodiments, the computing system may identify the activity towards achieving the endpoint based on a user profile (e.g., the user profile 160) or other indications (e.g., the personal value, state), among others. In some embodiments, the computing system may identify the activity towards achieving the endpoint based on response data subsequent to performing at least the first activity. With the identification of the activity, the computing system may select the configuration file with the set of content items to present.

The method 2000 may include displaying, rendering, or otherwise presenting the set of content items (2020). From the configuration file, the computing system may identify the one or more content items for prompting the user to perform the activity. The computing system may present the set of content items as identified from the configuration file. The set of content items may identify a first activity (when presented at a first-time instance to the user) or respective second activities (when presented to the user at time instances subsequent to the first-time instance). The computing system may monitor for one or more interactions by the user with the content items in performing the prompted activity. The presentation and the functionalities of the content items may be as described above in Section A.

In some embodiments, the computing system may prompt the user to provide an assessed rating in conjunction with the performance of the activity identified in the set of content items. The computing system may receive a rating prior to the performance of the activity and another rating subsequent to the performance of the activity. The computing system may determine a comparison between the ratings before and after the performance of the activity. With the receipt, the computing system may present the comparison of the ratings before and after the performance of the activity to the user.

The method 2000 may include retrieving, identifying, or otherwise obtaining a session metric (2025). The session metric may be obtained (e.g., by a clinician or the application 165) subsequent to the user performing one or more activities through the digital therapeutics applications. In some embodiments, the session metric may be obtained from a source separate from the application. For instance, a clinician examining the user may ascertain the session metric, and provide the session metric for storage. In some embodiments, a computing system (e.g., the application 165 or the application configuration service 105) may determine the session metric via the user interactions with a prompt (e.g., a questionnaire for the metric) presented through the user interface. The session metric may include, for example a score for one or more of the following: a Clinical Assessment Interview for Negative Symptoms (CAINS) Motivation and Pleasure Scale, Clinical Assessment Interview for Negative Symptoms, Expressivity Scale (CAINS-EXP), Positive and Negative Syndrome Scale (PANSS), Personal and Social Performance Scale (PSP), a Defeatist Beliefs Subscale of the Dysfunctional Attitudes Scale (DAS), Patient Global Impression of Improvement Scale (PGI-I), Patient Global Impression of Severity Scale (PGI-S), Clinical Global Impression of Severity Scale (CGI-S), WHO Disability Assessment Schedule 2.0 (WHODAS 2.0), or Schizophrenia Quality of Life Scale Revision 4 (SQLS-R4), among others. The session metric may be of the same type of metric as the baseline metric obtained prior to the performance of any activity.

In some embodiments, the computing system may identify, obtain, or otherwise receive response data (e.g., the response data 282). The response data may identify one or more interactions by the user with the set of content item. For example, at the end of the orientation phase, the digital therapeutics application may present the user with a questionnaire. This questionnaire may ask the user several questions related to recreation, socialization, and productivity, corresponding to the different classifications or domains for the endpoints. The computing system may use the response data identifying the answers to the questionnaire to recommend an upcoming domain. The application may provide the user the option to select the different domains, separate from the recommendations of the application. Using these inputs (assessment and the user selection of areas of focus), the application may select the endpoint that is personalized to the user to start their experience. Based on user success or failure with the goal, the application may continually keep personalizing the experience to fit the user at numerous points throughout their journey.

The method may include determining whether to continue (2030). The determination may be based on the set length (e.g., between 5 days and 25 weeks) of the trial or set number of endpoints, activities, or sessions (e.g., corresponding to individual time instances) to be provided to the user. When an amount of time from the obtaining of the baseline metric or the first activity has not exceeded the set length, the determination may be to continue and to repeat from (2010). In some embodiments, when the number of endpoints, activities, or sessions has exceeded the set number, the computing system may determine to continue and repeat from (2010). The computing system may repeat the determination of the endpoint (2010), the identification of a configuration file (2015), and the presentation of the set of content items (2020), among others, over a set of time instances (sometimes herein referred to as sessions). The presentations of the content items identifying the selected activities may be repeated until the determination to stop. Otherwise, when the amount of time from the obtaining of the baseline metric or the first activity has exceeded the set length, the determination may be to stop. In some embodiments, when the number of endpoints, activities, or sessions has not exceeded the set number, the computing system may determine to stop.

The method 2000 may include identifying determining whether the session metric is less than the baseline metric, when the determination is to stop (2030). To determine, the computing system may compare the baseline metric from prior to the first activity with the session metric obtained after performing of one or more subsequent activities (e.g., at or toward end of set time length). In some embodiments, the session metric compared against may be from the last of the time instances of the repeated presentations of the content items (e.g., after determination to stop). In some embodiments, the session metric compared against may be from at least one of the time instances of the repeated presentations of the content items (e.g., independent of the determination to continue or stop).

The method 2000 may include identifying or determining whether the session metric is an improvement over the baseline metric (2035). The improvement may correspond to an amelioration of experiential negative symptoms of schizophrenia. The improvement may correspond to when the session metric is statistically different (e.g., by a statistically significant margin) from the baseline metric. The improvement may be shown when the session metric is increased compared to the baseline metric by a first predetermined margin or when the session metric is decreased compared to the baseline metric by a second predetermined margin. The margin may identify or define a difference in value between the baseline and session metrics at which to determine that the user shows improvement in the degree of experiential negative symptoms of schizophrenia. Whether the improvement is shown by increase or decrease may depend on the type of metric used to measure the user with respect to the experiential negative symptoms of schizophrenia. The margin may also depend on the type of metric used and may in general correspond to the difference in value showing noticeable difference by the clinician or user with respect to the degree of experiential negative symptoms of schizophrenia, or showing a statistically significant result in the difference in the values between the baseline and session metrics.

The method 2000 may include determining that amelioration is shown when the session metric is determined to be an improvement over the baseline metric (2040). In some embodiments, the amelioration may be determined (e.g., by the computing system or a clinician examining the user) to occur when the session CAINS-MAP metric is decreased from the baseline CAINS-MAP metric by the second predetermined margin. In some embodiments, the amelioration may be determined to occur when the session CAINS-EXP metric is decreased from the baseline CAINS-EXP metric by the second predetermined margin. In some embodiments, the amelioration may be determined to occur when the session PANSS metric is decreased from the baseline PANSS metric by the second predetermined margin.

In some embodiments, the amelioration may be determined to occur when the session PSP metric is increased from the baseline PSP metric by the first predetermined margin. In some embodiments, the amelioration may be determined to occur when the session DAS metric is decreased from the baseline DAS metric by the second predetermined margin. In some embodiments, the amelioration may be determined to occur when the session CGI-S metric is decreased from the baseline CGI-S metric by the second predetermined margin. In some embodiments, the amelioration may be determined to occur when the session PGI-I metric is decreased from the baseline PGI-I metric by the second predetermined margin. In some embodiments, the amelioration may be determined to occur when the session PGI-S metric is decreased from the baseline PGI-S metric by the second predetermined margin.

In some embodiments, the amelioration may be determined to occur when the session EQ-5D-5L metric is increased from the baseline EQ-5D-5L metric by the first predetermined margin. In some embodiments, the amelioration may be determined to occur when the session SDS metric is increased from the baseline SDS metric by the first predetermined margin. In some embodiments, the amelioration may be determined to occur when the session WHODAS metric is decreased from the baseline WHODAS metric by the second predetermined margin. In some embodiments, the amelioration may be determined to occur when the session SQLS-R4 metric is decreased from the baseline SQLS-R4 metric by the second predetermined margin.

The method 2000 may include determining that amelioration is not shown when the session metric is determined to be not an improvement over the baseline metric (2045). In some embodiments, the amelioration may be determined (e.g., by the computing system or a clinician examining the user) to not occur when the session CAINS-MAP metric is not decreased from the baseline CAINS-MAP metric by the second predetermined margin. In some embodiments, the amelioration may be determined to not occur when the session CAINS-EXP metric is not decreased from the baseline CAINS-EXP metric by the second predetermined margin. In some embodiments, the amelioration may be determined to not occur when the session PANSS metric is not decreased from the baseline PANSS metric by the second predetermined margin.

In some embodiments, the amelioration may be determined to not occur when the session PSP metric is not increased from the baseline PSP metric by the first predetermined margin. In some embodiments, the amelioration may be determined to not occur when the session DAS metric is not decreased from the baseline DAS metric by the second predetermined margin. In some embodiments, the amelioration may be determined to not occur when the session CGI-S metric is not decreased from the baseline CGI-S metric by the second predetermined margin. In some embodiments, the amelioration may be determined to not occur when the session PGI-I metric is not decreased from the baseline PGI-I metric by the second predetermined margin. In some embodiments, the amelioration may be determined to not occur when the session PGI-S metric is not decreased from the baseline PGI-S metric by the second predetermined margin.

In some embodiments, the amelioration may be determined to not occur when the session EQ-5D-5L metric is not increased from the baseline EQ-5D-5L metric by the first predetermined margin. In some embodiments, the amelioration may be determined to occur when the session SDS metric is not increased from the baseline SDS metric by the first predetermined margin. In some embodiments, the amelioration may be determined to not occur when the session WHODAS metric is not decreased from the baseline WHODAS metric by the second predetermined margin. In some embodiments, the amelioration may be determined to not occur when the session SQLS-R4 metric is not decreased from the baseline SQLS-R4 metric by the second predetermined margin.

In some embodiments, the method 2000 may include determining that the user shows an amelioration of experiential negative symptoms of schizophrenia (e.g., when the user is provided the trial in accordance with Example 1), when the session metric is less than the baseline metric. The amelioration of experiential negative symptoms of schizophrenia may correspond to a decrease in CAINS-MAP score or DAS score among others. Otherwise, the method 2000 may include determining that the user does not show amelioration of experiential negative symptoms of schizophrenia, when the session metric is greater than the baseline metric. In some embodiments, the method 2000 may include determining that the user shows amelioration, when the session metric is greater than the baseline metric. The amelioration of experiential negative symptoms of schizophrenia may correspond to an increase in PSP score, among others. In contrast, the method 2000 may include determining that the user does not show amelioration, when the session metric is equal or less than the baseline metric.

### Example 1: Multi-Center, Exploratory, Single-Arm Study to Evaluate the Feasibility and Acceptability of Treatment with an Abbreviated Version of Digital Therapeutics Application in Adults Diagnosed with Schizophrenia

### Synopsis

Indication: Adults with Negative Symptoms of Schizophrenia

Introduction: the digital therapeutic application (e.g., the application 165 or also described herein as CT-155 or Study App) was an investigational prescription digital therapeutic (DTx) that delivers an interactive, software-based intervention for negative symptoms of schizophrenia. During a DTx development lifecycle, iterations of the DTx may be scientifically evaluated in a user population that is clinically representative of the intended patient population. Data generated via this evaluation can be used to drive the modification and optimization of specific therapeutic components contained within a given DTx. The purpose of the proposed study was to evaluate the feasibility and acceptability of use of an abbreviated version of digital therapeutic application (the Study App) in adults with schizophrenia experiencing at least moderate experiential negative symptoms.

### Objectives

Primary objective was to explore feasibility and acceptability of abbreviated treatment with the digital therapeutic application.

Exploratory objectives were as follows:
to explore change in experiential negative symptoms from baseline to end of study
to explore change in social functioning from baseline to end of study
to explore change in defeatist beliefs from baseline to end of study
to explore compliance with daily Study App engagement
to explore the correlations from baseline to Week 7 between changes in experiential negative symptoms and baseline motivation and pleasure, baseline personal and social performance, baseline digital literacy, baseline cognitive functioning, changes in strength of digital working alliance, changes in defeatist beliefs
to explore the participant expectation of therapeutic benefit throughout 7 weeks of Study App use

### Study Endpoints

Primary Endpoint included:
   Feasibility and acceptability of the Study App, defined as:
   Participants ratings of Study App quality and satisfaction as measured by the Mobile App Rating Scale (MARS) at Week 7
   Participant feedback captured in follow-up participant qualitative interviews and the Human Factors Questionnaire (HFQ)
Exploratory Endpoints included:
   Change in experiential negative symptoms from baseline to Week 7 as assessed by Clinical Assessment Interview for Negative Symptoms (CAINS) Motivation and Pleasure Scale
   Change in social functioning from baseline to Week 7 as assessed by Personal and Social Performance Scale (PSP)
   Change in defeatist beliefs between baseline and Week 7 as assessed by the Defeatist Beliefs Subscale of the Dysfunctional Attitudes Scale (DAS)
   Compliance with daily Study App engagement from baseline to Week 7
   Correlations from baseline to Week 7 between changes in experiential negative symptoms and baseline motivation and pleasure, baseline personal and social performance, baseline digital literacy, baseline cognitive functioning, changes in strength of digital working alliance, and changes in defeatist beliefs
   Expectation of therapeutic benefit of the Study App as assessed by the Expectation of Benefit Questionnaire (EBQ)
   Degree of participant engagement with the Study App as measured by participant app use data captured in-app

Study Design: This was a multi-center, exploratory, single-arm study to evaluate the feasibility and acceptability of treatment with an abbreviated version of the digital therapeutic application in adults diagnosed with schizophrenia. Eligible participants must have a diagnosis of schizophrenia per the Diagnostic and Statistical Manual of Mental Disorders, 5th Edition (DSM-5) and experience at least moderate to severe negative symptom severity as evidenced by a score of ≤30 on the Motivation and Pleasure Scale-Self Report (MAP-SR). Participants must be on a stable dose of antipsychotic medication(s) for at least 12 weeks (3 months) prior to enrollment (Day 1). Participants that meet eligibility criteria may be enrolled in the study on Day 1.

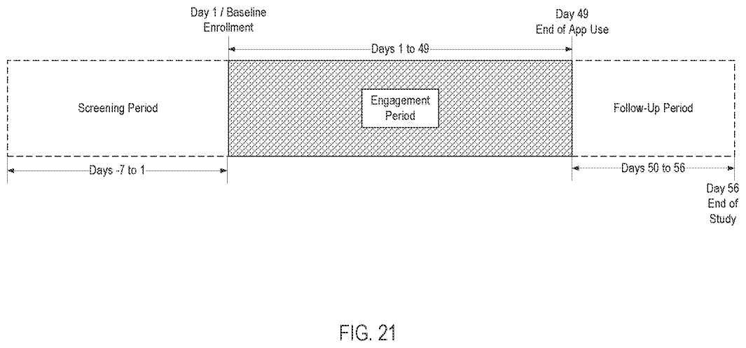

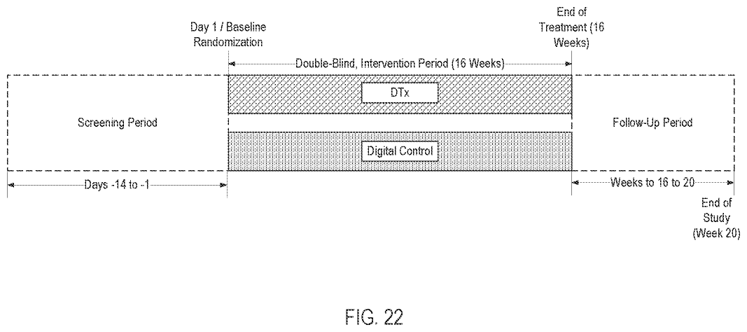

Referring now to FIG. 21, depicted is a study screen schema that was used to carry out the multi-center, exploratory, single-arm study to evaluate the feasibility and acceptability of treatment with an abbreviated version of the digital therapeutic application. As shown, the study included an up to 7-day screening period, a 49-day engagement period, and an up to 7-day follow-up period.

Screening Period (Day −7 to 1): All participants who have provided informed consent entered into an up to 7-day screening period to determine eligibility. Assessments during this period were performed during an in-person clinical visit. Site personnel introduced eligible participants to the Study App by assisting participants to download and install the Study App onto their personal primary iPhone or Android smartphone.

Engagement Period (Day 1 to 49): Eligible participants enrolled during an in-person clinic visit on Day 1. Assessments and activities during this period performed during in-person clinic visits according to the Schedule of Activities and Assessments (SoA). Participants were directed to access and perform tasks every day as directed by the Study App.

Follow-up Period (Day 50 to Day 56): Participants entered into an up to 7-day follow-up period in which participants may attend an in-person clinic visit to complete follow-up assessments according to the SoA. Participants may not perform any activities within the application.

Planned Number of Participants included: Up to 48 participants may be enrolled in this study.

### Study Entry Criteria

Inclusion Criteria included:

A participant was defined as eligible for entry into the study if all of the following criteria are met:
1. Is willing and able to provide written informed consent to participate in the study, attend study visits, and comply with study-related requirements and assessments.
2. Is between 18 and 64 years of age at the time of informed consent.
3. Is fluent in written and spoken English, confirmed by ability to read and understand the informed consent form.
4. Has a primary diagnosis of schizophrenia using the diagnostic criteria for schizophrenia as defined in the Diagnostic and Statistical Manual of Mental Disorders, 5th Edition (DSM-5), for at least 1-year prior to screening.
5. Is in the stable phase of illness, as assessed by the investigator after review of medical records or documented discussion with the treating physician.
6. Has outpatient treatment status at the time of screening, with no inpatient treatment for schizophrenia within 12 weeks prior to screening.
7. Is on a stable dose of antipsychotic medication(s) for at least 12 weeks prior to enrollment (Day 1) as determined by the investigator.
8. Has obtained a score of 30 or less on the MAP-SR as assessed at the screening visit.
9. Is the sole user, per participant self-report, of an iPhone with an iPhone operating system (iOS) 13 or greater or a smartphone with an Android operating system 10 or greater and is willing to download and use the Study App as required per the protocol.
10. Is the owner of, and has regular access to, an email address.
11. Has regular access to the internet via cellular data plan and/or Wi-Fi.
12. Has stable housing and has remained at the same residence for at least 12 weeks prior to screening, with no anticipated housing changes during the duration of the study.
13. Understands how to use the Study App during the screening visit as assessed by the investigator during in-clinic Study App installation and activation activities.

### Exclusion Criteria:

A participant was determined to be not eligible for study entry if any of the following criteria are met:
1. Is currently treated with more than two antipsychotic medications (including more than two dosage forms).
2. Is currently treated with clozapine or haloperidol.
3. Has active prominent positive symptoms in the opinion of the investigator that would preclude effective engagement in treatment for negative symptoms.
4. Is currently receiving or has received psychotherapy within 12 weeks prior to screening.
5. Meets either the International Classification of Diseases, Tenth Revision (ICD-10) or DSM-5 criteria for diagnoses not under investigation, including schizophreniform, schizoaffective, or psychosis non-specific disorders.
6. Has post-traumatic stress disorder (PTSD), bipolar disorder, major depressive disorder, developmental disorders, or any prominent disorder that would interfere with compliance to the protocol, per investigator judgment.
7. Has substance or alcohol use disorder (excluding caffeine and nicotine), that would interfere with compliance to the protocol, per investigator judgment.
8. Currently needs or will likely require prohibited concomitant medications and/or therapy during the study, as determined by the investigator.
9. Is currently participating in another clinical study (interventional or observational) involving investigational drugs or devices.
10. Has prior participation in the clinical study.
11. Has suicidal ideation or behavior, as assessed by the Columbia-Suicide Severity Rating Scale (C-SSRS):
   a. Participants with a “yes” response to either Items 4 or 5 on the C-SSRS Suicidal Ideation Item within the last 12 weeks prior to screening or at baseline visit.
   b. Participants with a “yes” response on the C-SSRS Suicidal Behavior Items within the last 26 weeks prior to screening or at baseline visit.
   c. Participants who, in the opinion of the investigator, present a serious risk of suicide.

12. In the judgment of the investigator, any evidence of a clinically significant concomitant disease or any other clinical condition that would jeopardize the participant's safety while participating in the clinical study.

Test Product, and Mode of Administration: During the screening visit, site personnel assisted eligible participants to download and install the Study App onto their own smartphone device. Participants activated the engagement module of the app at their baseline visit (Day 1).

Study Duration: The duration of participation was approximately 9 weeks as follows:
Screening Period: up to 7 days
Engagement Period: 49 days
Follow-up Period: up to 7 days
Sample Size: Up to 48 participants may be enrolled in the study

Statistical Analysis: A statistical analysis plan (SAP) that elaborates on the study objectives and analysis of endpoints included:
Degree of participant engagement with the Study App as measured by predefined Study App engagement metrics may be evaluated.
Qualitative analysis of participant feedback captured in follow-up participant qualitative interviews and the Human Factors Questionnaire (HFQ)
Change in strength of digital working alliance from baseline to Week 7 as assessed by the Mobile Agnew Relationships Measure (mARM)
A degree of overall negative schizophrenia symptomology as assessed by CAINS will be evaluated.
The degree of negative schizophrenia symptomology will be evaluated at Week 7 as assessed by CAINS.
The degree of improvement in negative schizophrenia symptomology from Baseline to Week 7 will be assessed by CAINS as a mean change.
The expectation of therapeutic benefit in people with more severe and moderate negative symptoms as assessed by EBQ will be evaluated.
The change from baseline to Week 7 in defeatist beliefs as assessed by DAS will be evaluated as a mean change.
The correlation between use of the Study App and strength of negative schizophrenia symptomology as assessed by predefined Study App engagement metrics, and CAINS will be evaluated.
The degree of relationship between baseline level of experiential negative symptoms as assessed by CAINS, baseline social functioning as assessed by PSP, baseline digital literacy as assessed by MDPQ, baseline cognitive functioning as assessed by BACS and the Altoida App, degree of strength of digital working alliance as assessed by mARM, and degree of expectation of therapeutic benefit will be evaluated through correlation coefficient.
The correlation between engagement with the Study App and expectation of benefit as assessed by predefined study app engagement metrics, and EBQ will be evaluated.

The digital therapeutics application is a novel prescription digital therapeutic (DTx) that delivers an interactive, software-based intervention to treat experiential negative symptoms in patients with schizophrenia who are stable on a standard-of-care antipsychotic medication (SOC). Therapeutic techniques for the digital therapeutics application were selected based on clinical evidence, and the individual components were designed based on the underlying principles of face-to-face treatment to provide maximal support to adults with schizophrenia. The digital therapeutics application therapeutic techniques are shown to work synergistically to produce an effect on experiential negative symptoms.

During a DTx development lifecycle, iterations of the DTx may be scientifically evaluated in a user population that is clinically representative of the intended patient population. Data generated via this evaluation can be used to drive the modification and optimization of specific therapeutic components contained within a given DTx.

The purpose of the proposed study was to evaluate the feasibility and acceptability of treatment with an abbreviated version of digital therapeutics application (the Study App) in adult participants with schizophrenia experiencing at least moderate experiential negative symptoms.

### Digital Therapeutic Application

Treatment guidelines for schizophrenia recommend antipsychotic medications and adjunctive psychosocial intervention. As previously noted, medications are efficacious in treating positive symptoms, but no pharmacological intervention is available for the treatment of negative symptoms. Therefore, adjunctive psychosocial intervention is recommended for symptoms not well treated by pharmacological intervention such as negative symptoms. The application (e.g., the application 165) delivers therapeutic techniques which translate the underlying principles of face-to-face psychosocial therapy to digital therapy and does so in a way that abides by the most recent evidence-based recommendations.

The digital therapeutics application is an adjunctive digital therapeutic designed to target experiential negative symptoms in people with schizophrenia who are stable on SOC. The digital therapeutics application will only be accessible via prescription and is intended to be used only under the supervision of a clinician. Given the age when schizophrenia emerges, it is clinically important that the product be evaluated and labeled for use in adults (18 to 64 years of age).

The digital therapeutics application was designed to address the need for an accessible treatment that can fill existing gaps in the SOC. Research has shown near ubiquity in smartphone ownership, with over 80% of those with schizophrenia reporting that they own a smartphone. Despite the absence of available validated mobile treatments, the majority of people with schizophrenia are already using technology to help manage their illness, from coping with hearing voices to setting medication reminders. The digital therapeutics application provided people with schizophrenia access to a validated digital treatment that augments their ongoing care.

The Study App was designed to provide an abbreviated version of a full treatment cycle with a digital therapeutics application. This design allowed inferences to be made about the usability and acceptability of digital therapeutics application but does not require full treatment with digital therapeutics application.

Objective included: To evaluate the interaction with therapeutic lesson and skill practice, and adaptive goals setting (AGS) activities in late adolescents and adults living schizophrenia and experiential negative symptoms (ENS).

### Investigational Device Under Evaluation

### Digital Therapeutics Application (Study App)

The digital therapeutics application (also referred herein as the Study App) was developed as a prescription adjunctive digital therapeutic designed to target experiential negative symptoms in adults and late adolescents with schizophrenia who are stable on a SOC. Therapeutic delivery is guided by a validated mechanistic model to address important clinical needs identified by people with schizophrenia. Therapeutic techniques in the application were selected based on clinical evidence, and the individual components were designed based on the underlying principles of face-to-face treatment to provide maximal support to people with schizophrenia. In addition, messages supporting therapeutic delivery, engagement, and adherence were sent throughout an individual's engagement with the application. The therapeutic techniques of the application worked synergistically to produce an effect on experiential negative symptoms.

### Handling and Storage of Application

### Study App Download

During the screening visit, site personnel assisted the participants to download and install the app. Instructions for installation were found in the Study App Site Instructions. Site personnel confirmed the Study App has been downloaded in the eCRF.

### Study App Activation

During the baseline visit, site personnel assisted the participant to activate the app. Instructions for activation were found in the Study App Site Instructions. Site personnel confirmed the Study App has been activated in the eCRF. Only participants who were confirmed as eligible and enrolled in the study were permitted to activate the Study App.

### Study App Deactivation and Uninstallation

After the end of the Week 7 visit (on Day 49) the Study App was automatically deactivated and became unusable for participants. Site personnel instructed participants who completed the study or terminated early to uninstall the Study App. Instructions for uninstallation were found in the Study App Site Instructions. Site personnel confirmed this instruction was provided to the participant in the eCRF.

### Study App Compliance

Participants were told to use the Study App as instructed by the Study App. Participants were presented with daily tasks, activities, and/or missions. Compliance with this regimen was not defined for this study. However, level of Study App engagement was measured.

Continued Access to Study App after the End of the Study

After completion of the engagement period (Day 49), the Study App became inert. After Day 49, participants did not have continued access to the content provided by the Study App during Days 1 to 49.

### Concomitant Therapy

Participants must have been on a stable dose of SOC antipsychotic medication for at least 12 weeks prior to enrollment (Day 1). Dose adjustments were permitted during the study, as outlined within the respective package inserts of their current medication(s).

Participants were not allowed to be treated with more than two (2) antipsychotic medications (including more than two dosage forms). Treatment with clozapine or haloperidol was prohibited. Any additional psychotherapy was not allowed during the study.

### Lifestyle Considerations

Participants had routine access to their smartphones for the duration of the trial. In addition, they should have been able to attend in-clinic visits during the trial. Participants were to refrain from using alcohol and recreational drugs during the times the Study App was accessed.

### Overall Design of Study

This was a multi-center, exploratory, single-arm study to evaluate the overall effects of use of an abbreviated version of digital therapeutics application in adults diagnosed with schizophrenia. Eligible participants must have had a diagnosis of schizophrenia per the Diagnostic and Statistical Manual of Mental Disorders, 5th Edition (DSM-5) and have experienced at least moderate to severe negative symptom severity as evidenced by a score of ≤30 on the Motivation and Pleasure Scale-Self Report (MAP-SR). Participants must have been on a stable dose of antipsychotic medication(s) for 12 weeks (3 months) prior to enrollment (Day 1). Participants that met eligibility criteria were enrolled in the study on Day 1.

The duration of participation was approximately 9 weeks, including an up to 7-day screening period, a 49-day engagement period, and an up to 7-day follow-up period as depicted in FIG. 21.

The activities and assessments during the up to 7-day screening period, the 49-day engagement period, and the up to 7-day follow-up period were completed according to the Schedule of Activities and Assessments (SoA). Trial site staff implemented procedures during in-person clinic visits. Participants were assessed based on validated standard clinician-rated and participant-rated outcome scales for schizophrenia at screening and baseline. Participants were assessed based on both validated and qualitative participant-rated outcome scales to assess study endpoints during the engagement period and the follow-up period. Participants' engagement with the application (sometimes herein referred to as a “study app”) was evaluated based on data captured within the application. Participants were also evaluated for AEs throughout the duration of the trial. A trial site conducted an unscheduled visit in person or remotely via telephone call at any time if requested by the investigator or study participant, or to assess a safety concern.

### Screening Period (Day −7 to Day 1)

During an in-person screening visit, participants signed an informed consent form (ICF) and all activities and assessments listed in the SoA will be completed. After this initial screening visit, participants entered an up to 7-day screening period during which participants' eligibility and interest in the trial may continue to be assessed. The screening visit may, however, occur on the same day as baseline (Day 1).

Site personnel introduced eligible participants to the Study App by assisting participants to download and install the Study App onto the participants' personal iPhone or Android smartphone.

### Engagement Period (Day 1 to Day 49)

During an in-person baseline visit on Day 1, participant's eligibility was confirmed. Participants were considered eligible to receive the Study App if they continue to meet all inclusion and no exclusion criteria.

Up to 48 eligible participants were enrolled across approximately 15 study centers in the US. Activities and assessments during this period were performed in-clinic between the site personnel and the participant. All assessments were completed according to the SoA.

Upon enrollment, the Study App was activated using a unique activation code and site personnel may check that the Study App is functioning properly. During the 49-day engagement period, participants were directed to access and perform tasks every day as directed by the Study App.

### Follow-up Period (Day 50 to Day 56)

Participants will enter up to a 7-day follow-up period in which the participants may not use the Study App. Activities and assessments during this period were completed according to the SoA. Participants, on an individual basis, joined the product development team for a 60-minute qualitative interview to discuss their experience using the Study App via remote teleconference software. The week 8 follow-up visit may occur up to 6 days prior to Day 56.

### End of Study Definition

The end of the study was defined as the date of the last contact, or the date of final contact attempt, for the last participant completing or withdrawing from the trial.

Participants who are evaluated at their last scheduled visit (Day 49, Week 7) were defined as trial completers.

### Study Assessments and Procedures

Study assessments and procedures, including their timing, are summarized in the SoA. Adherence to the study design requirements, including those specified in the SoA, was essential and required for study conduct. Every effort should be made to ensure that the protocol required assessments and procedures are completed as described. All the clinician administered scales should be administered by individuals who have been appropriately trained. Study procedures are described herein. Study assessments are described below.

### Study Assessments and Scales

The following assessment scales were used in this study at the times as provided in the SoA.

### Study App Engagement

Participants' engagement with the Study App may be captured automatically by the study app. The metrics below were captured:
Number of available lessons completed out of the total lessons assigned within the Study App.
Number of days the Study App is opened out of total available days of treatment
Number of times the Study App is opened via notification click
Number of times the Study App is opened via SMS link
Number of daily check-ins completed out of total assigned
Overall percentage of assigned tasks that were completed.
Duration of each app use session
Number of times each skill is practiced
Treatment focus chosen by the participant
Percent of goals successfully achieved
Percent of goal-centered tasks user completes

### Motivation and Pleasure Scale-Self-Report (MAP-SR)

The MAP-SR is a validated self-report tool derived from the CAINS that assesses the motivation and pleasure domain of negative symptoms in patients with psychotic disorders. The scale consists of 15 items that record motivation, effort, interest, and pleasure in different areas of life. All items are rated on a 5-point Likert scale, where lower scores reflect greater severity.

### Clinical Assessment Interview for Negative Symptoms (CAINS)

The CAINS is a clinician-administered, validated 13-item interview-based assessment comprised of two subscales that measure the two factors of negative symptoms: Motivation and Pleasure (MAP) and Expressivity (EXP). CAINS items are scored on a 4-point scale, with lower scores indicating lower negative symptom severity.

The MAP scale included nine items that measure interest and engagement in motivated behavior as well as the experience of pleasure across social, vocational, and recreational domains. Each item is scored based on patient-reported behavior and experience. The EXP scale included four items that measure verbal intonation (prosody), non-verbal expressivity (gestures, posture), facial expressivity, and speech output (alogia). Items are rated based on observation over the course of the interview.

### Personal and Social Performance Scale (PSP)

The PSP is a validated clinician-rated scale that measures personal and social functioning in four domains: socially useful activities (e.g., work and study), personal and social relationships, self-care, and disturbing and aggressive behaviors. Each area is scored on a 0-100 scale, with anchors for every 10-point interval.

### Mobile Device Proficiency Questionnaire (MDPQ)

The MDPQ is a validated self-administered scale that measures mobile device proficiency in older adults. The questionnaire consists of 46 items that are rated on a 5-point Likert scale. The MDPQ assesses five aspects of digital literacy: information and data literacy; communication and collaboration; digital content creation; safety; and problem solving.

### Altoida Computerized Cognitive Assessment

The Altoida app is a validated computerized cognitive assessment providing digital biomarker data for cognition and functional abilities, including 13 neurocognitive domains (spanning everyday function and cognition), which correspond to the major neurocognitive networks, such as complex attention and cognitive processing speed. Nearly eight hundred (800) individual features, such as reaction time, speed, attention- and memory-based assessments, as well as every device sensor input (or lack thereof) through accelerometer, gyroscope, magnetoscope, camera, microphone, and touch screen are collected during augmented reality and motor tasks.

The assessment was completed by the participant on a study iPad in-clinic and takes 10 minutes.

### Brief Assessment of Cognition in Schizophrenia (BACS) Subscales

The Brief Assessment of Cognition in Schizophrenia (BACS) is a validated pen/paper cognitive assessment, containing several subtests, including symbol coding task assessing working memory; the verbal memory task; and the digit sequencing task, assessing attention and processing speed of information. These three subtests can be combined into a short-form global screener of cognitive functioning (BACS-SF).
Symbol coding task: Participants write numerals 1-9 matching to symbols on a response sheet for 90 seconds. Total task duration is 3 minutes.
Verbal memory task: Participants are presented with 15 words and then asked to recall as many as possible. Procedure is repeated 5 times. Total task duration is 7 minutes.
Digit Sequencing: Participants are presented with clusters of numbers of increasing length and are required to tell the experimenter the numbers in order, from lowest to highest. Total task duration is 5 minutes.

### Defeatist Performance Beliefs Subscale of the Dysfunctional Attitudes Scale (DAS)

The Defeatist Performance Beliefs subscale is a 15-item subset of the validated Dysfunctional Attitudes Scale. Items are scored by the participant on a 1-7 scale, with higher scores indicating more severe defeatist thinking.

### Expectation of Benefit Questionnaire (EBQ)

The EBQ is a non-validated participant-completed measure designed to assess the participant's expectations of receiving therapeutic benefit from use of the Study App. Participants rate three items on a 5-item scale ranging from “Strongly Disagree” to “Strongly Agree.”

Mobile Agnew Relationship Measure Questionnaire (mARM)

The mARM is a validated patient-rated scale that assesses mobile health interventions for mental health conditions. It was adapted from the well-validated Agnew Relationship Measure (ARM) and consists of 25 items that are rated on a 7-item scale that ranges from “Strongly Disagree” to “Strongly Agree.”

### Human Factors Questionnaire (HFQ)

The HFQ is a non-validated participant-completed measure containing 5 questions designed to assess the participant's experience using the Study App.

### Mobile Application Rating Scale (MARS)

The MARS is a validated 23-item scale that classifies and assesses the quality of mobile health apps. The scale assesses many aspects of an application: engagement, functionality, aesthetics, and information quality, and subjective quality. The mean score is calculated for each section of the scale, and the MARS total mean score describes the overall quality of an app.

### Qualitative Interview

Participants may individually complete a non-validated qualitative interview with members of the Product Development Team. During this interview participants may be asked for their feedback about the Study App.

### Results

Individual and mean CAINS-MAP baseline and end of study (EOS) (e.g., after 7-week treatment) using the digital therapeutics application were acquired. There was a significant decrease in the mean (95% CI) CAINS-MAP score of 3.4 (1.2, 5.7) or ˜17% (p=0.004) following 7 weeks of digital therapy app use indicated improved experiential negative symptoms (ENS) in patients. There was no correlation between baseline ENS and the number of sessions completed. From the CAINS-MAP baseline and EOS scores, it was evident that patients with schizophrenia demonstrated a reduction in experiential negative symptoms following 7 weeks of CT-155 beta version app engagement. Patients with severe experiential negative symptoms benefitted more consistently from the app compared to those with symptoms of milder severity.

### Example 2: Combination Therapy with Digital Therapeutics and Antipsychotics in Subjects with Experiential Negative Symptoms of Schizophrenia

### Synopsis

Indication: the digital therapeutics application (e.g., the application 165 or also described herein as CT-155 or Study App) is an investigational prescription digital therapeutic (PDT) indicated for the treatment of adults and late adolescents with experiential negative symptoms of schizophrenia (adjunct to standard-of-care antipsychotic therapy; SOC).

Introduction: the digital therapeutics application delivers an interactive, software-based intervention for experiential negative symptoms of schizophrenia. The purpose of the proposed study is to evaluate the efficacy of CT-155 as an adjunct treatment to SOC relative to a comparator Digital Control in participants aged 18 years or older diagnosed with experiential negative symptoms of schizophrenia.

Objective: To evaluate the efficacy of CT-155 in reducing experiential negative symptoms, compared with a Digital Control, in adult and late adolescent participants diagnosed with schizophrenia.

### Criteria for Evaluation

Primary Efficacy Endpoint
   Change from baseline to Week 16 in experiential negative symptoms, as assessed by Clinical Assessment Interview for Negative Symptoms, Motivation and Pleasure Scale (CAINS-MAP), as compared to a Digital Control
Secondary Efficacy Endpoints
   Change from baseline in motivation and pleasure symptoms at Week 8, as assessed by CAINS-MAP, as compared to Digital Control
   Change from baseline in expressive negative symptoms at Weeks 8 and 16, as assessed by the Clinical Assessment Interview for Negative Symptoms, Expressivity Scale (CAINS-EXP), as compared to Digital Control
   Change from baseline in positive symptoms at Weeks 8 and 16, as assessed by the Positive and Negative Syndrome Scale (PANSS), as compared to Digital Control
   Change from baseline in social functioning at Weeks 8 and 16, as assessed by the Personal and Social Performance Scale (PSP), as compared to Digital Control
   Change from baseline in self-reported defeatist beliefs at Weeks 8 and 16, as assessed by the Defeatist Beliefs Subscale of the Dysfunctional Attitudes Scale (DAS), as compared to Digital Control
   Patient Global Impression of Improvement Scale (PGI-I) at Weeks 8 and 16, as compared to Digital Control
Exploratory Endpoints
   Key engagement metrics
   Change from baseline in disease severity at Week 8 and 16, as assessed by the Clinical Global Impression of Severity Scale (CGI-S), as compared to Digital Control
   Change from baseline in disease severity at Week 8 and Week 16, as assessed by the WHO Disability Assessment Schedule 2.0 (WHODAS 2.0), as compared to Digital Control
   Change from baseline in disease severity at Week 8 and Week 16, as assessed by the Schizophrenia Quality of Life Scale-Revision 4 (SQLS-R4), as compared to Digital Control

Study Design: This will be a multicenter, randomized, double-blind, controlled trial to evaluate the efficacy and safety of CT-155 in adults and late adolescents diagnosed with schizophrenia. Eligible participants must have a diagnosis of schizophrenia per the Diagnostic and Statistical Manual of Mental Disorders, Fifth Edition (DSM-5), and must experience at least moderate to severe experiential negative symptom severity as evidenced by a score of ≥2 (moderate to severe) in at least two of the three CAINS-MAP domains (Social, Work, or Recreational). Participants must be on a stable dose of antipsychotic medication(s) for 12 weeks prior to randomization (Day 1). Participants who met eligibility criteria will be randomized to receive the intervention with CT-155 or a comparator Digital Control (the Study App) on Day 1.

Referring to FIG. 25, depicted is a study design schema for combination therapy with digital therapeutics and antipsychotics in subjects with experiential negative symptoms of schizophrenia. The study consists of an up to 14-day screening period, a 16-week double-blind intervention period, and a 4-week follow-up period.

### Study Design Schema

Screening Period (Day −14 to −1): All eligible participants (per investigator) will enter a screening period of up to 14 days (minimum 7 days) to determine eligibility. Participants who met all applicable inclusion criteria and no exclusion criteria at the time of the Screening Visit will be introduced to the Study App by downloading and installing the application onto their personal iPhone or Android smartphone.

Double-blind Intervention Period (Day 1 to Week 16): Approximately 432 eligible participants will be randomized in a 1:1 ratio (Digital Control) across approximately 40 study centers in the US during an in-person clinic visit on Day 1. Assessments and activities during this period will be performed during in-person clinic visits or remotely by telephone visits according to the SoA.

Follow-up Period (Week 16 to Week 20): Participants will enter a 4-week follow-up period in which the participants will not receive the randomized intervention. Assessments and activities during this period will be completed by way of a remote telephone visit according to the SoA.

Planned Number of Participants: Approximately 432 participants will be randomized in this study.

### Study Entry Criteria

Inclusion Criteria: A participant will be eligible for entry into the study if all of the following criteria are met:
1. Is willing and able to provide written informed consent to participate in the study, attend study visits, and comply with study-related requirements and assessments.
2. Adult or late adolescent, 18 years of age or older at the time of informed consent.
3. Fluent in written and spoken English, confirmed by ability to read and understand the informed consent form.
4. Has a primary diagnosis of schizophrenia using the diagnostic criteria for schizophrenia, as defined in the DSM-5, for at least 6 months prior to the Screening Visit.
5. Is in the stable phase of illness, as assessed by the investigator after review of medical records or documented discussion with the treating physician.
6. Has outpatient treatment status at the time of screening, with no inpatient treatment for schizophrenia within 12 weeks prior to screening.
7. Is on a stable dose of antipsychotic medication(s) for at least 12 weeks prior to randomization (Day 1), with dose adjustments permitted during the study (and the 12 weeks prior to randomization), as outlined within the respective package inserts of their current medication(s) as determined by the investigator.
8. Has obtained an average score of ≥2 (moderate to severe) in at least two of the three CAINS-MAP domains (Social, Work, or Recreational) at the Screening Visit and at baseline (Day 1).
9. Is the sole user of an iPhone with an iPhone operating system (iOS) 14 or greater, or a smartphone with an Android operating system (OS) 10 or greater and is willing to download and use the specified Study App required by the protocol.
10. Is willing and able to receive SMS text messages and push messages on their smartphone.
11. Is the owner of, and has regular access to, an email address.
12. Has regular access to the Internet via cellular data plan and/or wi-fi.
13. Has stable housing and has remained at the same residence for at least 12 weeks prior to screening, with no anticipated housing changes during the duration of the study.
14. Understanding of and interest in the use of the Study App during the screening period and the Baseline Visit (Day 1).

Exclusion Criteria: A participant will not be eligible for study entry if any of the following criteria are met:
1. Is currently treated with more than two antipsychotic medications (including more than two dosage forms).
2. Is currently treated with clozapine or haloperidol, or treated with clozapine within 5 years of the Screening Visit.
3. Has obtained a positive symptom item score of >4 (moderate) on P1 Delusions, P2-Disorganization, P3-Hallucinations, P6-Suspiciousness, or any item >5 (moderate-severe) at the Screening or Baseline Visit on the PANSS, thus indicating prominent positive symptoms.
4. Is currently receiving or has received psychotherapy, defined as individual or group-based structured treatment (e.g., Cognitive Behavioral Therapy, Social Skills Training, or Vocational/Occupational Therapy) within 6 months (26 weeks) prior to screening per Investigator assessment.
5. Meets DSM-5, for diagnoses not under investigation that will impact their compliance to the protocol, including schizophreniform, schizoaffective, or psychosis non-specific disorders (post-traumatic stress disorder [PTSD], bipolar disorder, major depressive disorder, or developmental disorders).
6. Meets criteria per DSM-5 for a current episode of depression, mania, or hypomania.
7. Has a current diagnosis of substance or alcohol use disorder (excluding caffeine and nicotine), as defined in DSM-5, within 6 months (26 weeks) of the Screening Visit.
8. Has a positive urine drug screen at screening or prior to randomization at the Baseline Visit for amphetamines (including MDMA/ecstasy), phencyclidine (PCP), cocaine, opiates, benzodiazepines, or barbiturates. Participants with a positive urine drug test and/or recreational use of THC may be recruited at the discretion of the investigator.
9. Has participated in a clinical study associated with the digital therapeutics application; or has participated in a user research study for the digital therapeutics application.
10. Has participated in another clinical study (interventional or observational) in the last 6 months (26 weeks).
11. Currently needs or will likely require prohibited concomitant medications and/or therapy during the trial, as determined by the investigator.
12. Has suicidal ideation or behavior, as assessed by the C-SSRS:
   a. Participants with a “yes” response to either Items 4 or 5 on the C-SSRS Suicidal Ideation Item within the last 3 months (12 weeks) prior to screening, or at the Baseline Visit.
   b. Participants with a “yes” response on the C-SSRS Suicidal Behavior Items within the last 6 months (26 weeks) prior to screening, or at the Baseline Visit.
13. Participants who, in the opinion of the investigator, present a serious risk of suicide.

Test Product, and Mode of Administration: Eligible participants will download and install the Study App onto their own smartphone at the Screening Visit. Participants will be randomized to the digital therapeutics application or a Digital Control at the Baseline Visit (Day 1).

Study Duration: The duration of participation will be approximately 22 weeks as follows:
Screening Period: up to 14 days
Intervention Period: 16 weeks
Follow-up Period: 4 weeks

Sample Size: Approximately 432 participants will be randomized to the study.

Statistical Analysis: The primary endpoint is change from baseline to Week 16 in experiential negative symptoms, as assessed by CAINS-MAP scale. The null hypothesis is that there is no difference between the means of the treatment arms and the alternative is that the means are different. In order to demonstrate this, and assuming a standardized effect (Cohen's d) between the treatment arms of 0.35, 173 participants per arm are required to achieve 90% power when using a type I error rate of 5% (two-sided). Assuming 20% early termination, 432 participants will need to be randomized to the study in a 1:1 ratio (approximately 216 per arm). Sample size was calculated using t-test for two independent samples and assuming equal variance.

The primary endpoint of change from baseline to Week 16 in experiential negative symptoms will be analyzed using Mixed Models Repeated Measures (MMRM) with response variable of change from baseline at each visit in which the CAINS-MAP is evaluated. The model will include an intercept and the following covariates: experiential negative symptom at baseline, visit (as class variable), treatment arm, treatment by visit interaction term, and baseline by visit interaction term.

The first choice for the within-patient correlations will be an unstructured covariance matrix. Mitigation in the case of no convergence will be discussed in the statistical analysis plan (SAP). Only data at scheduled visits will be used for this analysis. The study will be considered successful if the two-sided p-value for the difference in the change from baseline at Week 16 between the two treatment arms is smaller than 0.05 and a larger decrease in CAINS-MAP is observed in the digital therapeutics application arm than the Digital Control arm. The analysis of the secondary endpoint will be analyzed in a similar manner using the MMRM. Exploratory endpoints will be summarized descriptively by treatment arm.

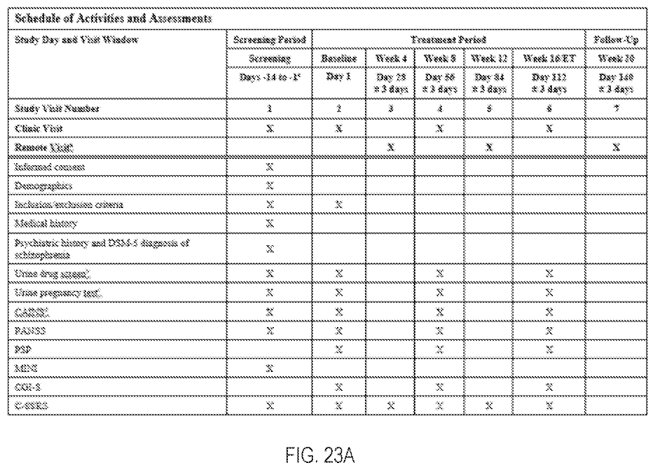

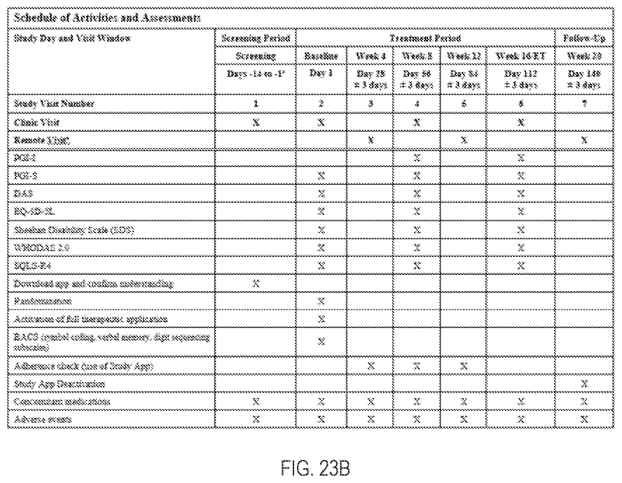

FIGS. 23A and 23B depict schedule of activities and assessments for participants. Abbreviations: app=application; BACS=Brief Assessment of Cognition in Schizophrenia; C-SSRS=Columbia-Suicide Severity Rating Scale; CAINS=Clinical Assessment Interview For Negative Symptoms; CGI-S=Clinician Global Impression of Severity; DAS=Defeatist Beliefs Subscale of the Dysfunctional Attitudes Scale; DSM-5=Diagnostic and Statistical Manual of Mental Disorders, Fifth Edition; EQ-5D-5L=EuroQol 5-Dimension 5-Level Scale; ET=early termination; MINI=Mini International Neuropsychiatric Interview; PANSS=Positive and Negative Syndrome Scale; PGI-I=Patient Global Impression of Improvement; PGI-S=Patient Global Impression of Severity; PSP=Personal and Social Performance Scale; SDS=Sheehan Disability Scale; SQLS-R4=Schizophrenia Quality of Life Scale-Revision 4; WHODAS=World Health Organization Disability Assessment Schedule 2.0.
a. All remote visits will be conducted via telephone.
b. The urine drug screen will be conducted on-site.
c. The urine pregnancy test will be conducted on-site using the dipstick method.
d. CAINS is administered by a centralized blinded rater team.

### Screening Period is a Minimum of Seven (7) Days Before the Baseline Visit.

### Digital Therapeutics Application Under Study

Current treatment guidelines for schizophrenia recommend antipsychotic medications and adjunctive psychosocial intervention. As previously noted, medications are efficacious in treating positive symptoms, but no pharmacological intervention is available for the treatment of negative symptoms. Therefore, adjunctive psychosocial intervention is recommended for symptoms not well treated by pharmacological intervention such as negative symptoms. The digital therapeutics application delivers therapeutic techniques which translate the underlying principles of face-to-face psychosocial therapy to digital therapy and does so in a way that abides by the most recent evidence-based recommendations. The digital therapeutics application provides an adaptive framework for supporting participants in building the skills they need to make progress towards personalized goals in their daily lives.

The digital therapeutics application is an adjunctive digital therapeutic designed to target experiential negative symptoms in adult and late adolescent patients with schizophrenia who are stable on SOC. The digital therapeutics application will only be accessible via prescription and is intended to be used only under the supervision of a clinician. Given the age when schizophrenia emerges, it is clinically important that the product be evaluated and labeled for use in both late adolescents (18 to 21 years of age) and adults (22 to 64 years of age)

The digital therapeutics application is designed to address the need for an accessible treatment that can fill existing gaps in the SOC. Recent research has shown near ubiquity in smartphone ownership, with over 80% of those with schizophrenia reporting that they own a smartphone. Despite the absence of available validated mobile treatments, the majority of people with schizophrenia are already using technology to help manage their illness, from coping with hearing voices to setting medication reminders. The digital therapeutics application will provide adult and late adolescent patients with schizophrenia access to a validated digital treatment that augments their ongoing care.

### Benefit/Risk Assessment

### Risk Assessment

No adverse events (AEs) are anticipated specific to digital therapeutics application or comparator Digital Control, due to the software-only nature of both interventions. The primary potential risk to the participant that could lead to AEs is the worsening of negative symptoms of schizophrenia. There is also risk of injury when doing the practice activities outside of the Study App as directed. These and other potential risks may be considered minimal, and no greater than those associated with SOC behavioral therapies for the treatment of schizophrenia.

The participants may experience some AEs due to their underlying condition or with the use of adjunctive treatment. The risk profile of SOC used in clinical practice is well understood and is detailed in their respective package inserts.

### Benefit Assessment

Trial participants may receive direct benefit from the interactive, software-based intervention featuring cognitive training and messaging. The digital therapeutics application is an adaptation of cognitive behavioral therapy (CBT), which is well-validated as a therapeutic option for negative symptoms associated with schizophrenia. It integrates multiple neurobehavioral therapeutic techniques that work together to reverse the negative symptoms associated with schizophrenia, as described in the psychological model of negative symptomatology.

### Overall Benefit: Risk Conclusion

The digital therapeutics application potentially delivers therapeutic efficacy with non-significant risk. The digital therapeutics application may improve experiential negative symptoms.

### Study Design

### Overall Design

This is a multicenter, randomized, double-blind, controlled trial to evaluate the efficacy and safety of digital therapeutics application in adults and late adolescents diagnosed with schizophrenia. Eligible participants must have a diagnosis of schizophrenia per the Diagnostic and Statistical Manual of Mental Disorders, Fifth Edition (DSM-5), and experience at least moderate to severe negative symptom severity as evidenced by a score of ≥2 (moderate to severe) in at least two of the three CAINS-MAP domains (Social, Work, or Recreational). Participants must be on a stable dose of antipsychotic medication(s) for 12 weeks prior to randomization (Day 1). Participants who meet eligibility criteria will be randomized to receive intervention with digital therapeutics application or comparator Digital Control (the Study App) on Day 1. Trial participants will be blinded to the hypothesis and treatment assignment and informed that they will receive one of the two digital interventions being studied. The duration of participation will be approximately 22 weeks, including an up to 2-week screening period, a 16-week double-blind intervention period, and a 4-week follow-up period.

To mitigate participant expectation, participants in this trial will be blinded to the efficacy hypothesis and their treatment assignment. Eligible participants will be informed by trial site staff that a) they will participate in the trial for up to 22 weeks (including the follow-up period) and will be randomized to one of two digital therapeutic treatments and b) the purpose of the trial is to compare the effectiveness of these two digital therapeutic treatments when used in addition to SOC. Both treatment arms will be presented as possibly helping to improve schizophrenia. No references to digital therapeutics application or Digital Control should be made to the participant; both should only be referred to as the Study App.

The activities and assessments during the up to 2-week screening period, the 16-week double-blind intervention period, and the 4-week follow-up period will be completed according to the Schedule of Activities and Assessments (SoA). Trial site staff will implement procedures either during in-person clinic visits or remotely by telephone visit. Participants will be assessed based on validated standard clinician-rated and participant-rated outcome scales for schizophrenia at screening, the intervention period, and the follow-up period. Participants will also be evaluated for safety throughout the duration of the trial. A trial site may conduct an unscheduled visit in person or remotely at any time if needed to assess a safety issue/concern.

### Screening Period (Day −14 to Day −1)

During an in-person Screening Visit, participants will sign an informed consent form and all assessments and activities listed in the SoA will be completed.

Unblinded site personnel (other than the investigator/assessor) will introduce eligible participants to the Study App by downloading and installing the Study App software onto the participants' personal iPhone or Android smartphone. Participants will complete various assignments over the course of at least seven days within the Study App's onboarding module to confirm understanding of and interest in the trial and use of the Study App.

### Double-Blind Intervention Period (Day 1-Week 16)

During an in-person visit on Day 1, participant eligibility will be confirmed. Participants will be considered eligible for randomization based upon the following:

Continuing to meet all inclusion and no exclusion criteria based on investigator assessment.

Approximately 432 eligible participants will be randomized in a 1:1 ratio (CT-155: Digital Control) across approximately 40 study centers in the U.S. Assessments and activities during this period will be performed during in-person clinic visits or remotely by telemedicine technology visits. All assessments will be completed according to the SoA.

Upon randomization, during the baseline visit, unblinded trial site staff will assist the participant to activate the assigned software module (digital therapeutics application (Study App) or Digital Control) within the Study App and confirm that it is functioning properly.

During the 16-week intervention period, the Study App will instruct participants to access and perform tasks at about the same time every day.

The efficacy assessment scales are described in detail. The CAINS efficacy assessments will be administered by a centralized blinded rater team.

To mitigate the risk of unblinding, trial site staff will instruct participants not to discuss what they are doing and seeing in the Study App.

### Follow-Up Period (Week 16-Week 20)

After the end of Week 16, the Study App will no longer actively prompt the participant. Participants will enter a 4-week follow-up period in which the participants will not receive randomized intervention. Assessments and activities during this period will be completed by way of a remote telephone visit according to the SoA. After Week 20, the app will become inert.

At the conclusion of a participant's participation in the trial, and after all final visit procedures have been completed, trial site staff will inform the participant of the trial hypothesis (i.e., that one digital therapeutic was hypothesized to be more beneficial in improving negative symptoms of schizophrenia), but there was a need for a trial to confirm. Trial site staff will be provided with debriefing guidelines to assist in this discussion with the participant.

### End of Study Definition

The end of the study is defined as the date of the last contact, or the date of final contact attempt, for the last participant completing or withdrawing from the trial.

For the purposes of this trial, participants who complete the trial assessments at Day 112 (Week 16) will be defined as trial completers.

### Study Population

Eligible participants who complete informed consent will be assigned a unique participant identification number at screening. Prospective approval of protocol deviations to recruitment and enrollment criteria, also known as protocol waivers or exemptions, is not permitted.

### Inclusion Criteria

A participant will be eligible for entry into the study if all of the following inclusion criteria are met:
1. Is willing and able to provide written informed consent to participate in the study, attend study visits, and comply with study-related requirements and assessments.
2. Adult or late adolescent, 18 years of age or older at the time of informed consent.
3. Fluent in written and spoken English, confirmed by ability to read and understand the informed consent form.
4. Has a primary diagnosis of schizophrenia using the diagnostic criteria for schizophrenia, as defined in the DSM-5, for at least 6 months prior to the Screening Visit.
5. Is in the stable phase of illness, as assessed by the investigator after review of medical records or documented discussion with the treating physician.
6. Has outpatient treatment status at the time of screening, with no inpatient treatment for schizophrenia within 12 weeks prior to screening.
7. Is on a stable dose of antipsychotic medication(s) for at least 12 weeks prior to randomization (Day 1), with dose adjustments permitted during the study (and the 12 weeks prior to randomization), as outlined within the respective package inserts of their current medication(s) as determined by the investigator.
8. Has obtained an average score of ≥2 (moderate to severe) in at least two of the three CAINS-MAP domains (Social, Work, or Recreational) at the Screening Visit and at baseline (Day 1).
9. Is the sole user of an iPhone with an iPhone operating system (iOS) 14 or greater, or a smartphone with an Android operating system (OS) 10 or greater and is willing to download and use the specified Study App required by the protocol.
10. Is willing and able to receive SMS text messages and push messages on their smartphone.
11. Is the owner of, and has regular access to, an email address.
12. Has regular access to the Internet via cellular data plan and/or wi-fi.
13. Has stable housing and has remained at the same residence for at least 12 weeks prior to screening, with no anticipated housing changes during the duration of the study.
14. Understanding of and interest in the use of the Study App during the screening period and the Baseline Visit (Day 1).

Exclusion Criteria: A participant will not be eligible for study entry if any of the following exclusion criteria are met:
1. Is currently treated with more than two antipsychotic medications (including more than two dosage forms).
2. Is currently treated with clozapine or haloperidol, or treated with clozapine within 5 years of the Screening Visit.
3. Has obtained a positive symptom item score of >4 (moderate) on P1-Delusions, P2-Disorganization, P3-Hallucinations, P6-Suspiciousness, or any item >5 (moderate-severe) at the Screening or Baseline Visit on the PANSS, thus indicating prominent positive symptoms.
4. Is currently receiving or has received psychotherapy, defined as individual or group-based structured treatment (e.g., Cognitive Behavioral Therapy, Social Skills Training, or Vocational/Occupational Therapy), within 6 months (26 weeks) prior to screening per investigator assessment.
5. Meets DSM-5, for diagnoses not under investigation that will impact their compliance to the protocol, including schizophreniform, schizoaffective, or psychosis non-specific disorders (post-traumatic stress disorder [PTSD], bipolar disorder, major depressive disorder, or developmental disorders).
6. Meets criteria per DSM-5 for a current episode of depression, mania, or hypomania.
7. Has a current diagnosis of substance or alcohol use disorder (excluding caffeine and nicotine), as defined in the DSM-5, within 6 months (26 weeks) of the Screening Visit.
8. Has a positive urine drug screen at screening or prior to randomization at the Baseline Visit for amphetamines (including MDMA/ecstasy), phencyclidine (PCP), cocaine, opiates, benzodiazepines, or barbiturates. Participants with a positive urine drug test and/or recreational use of THC may be recruited at the discretion of the investigator.
9. Has participated in a prior digital therapeutics application clinical study; or has participated in a digital therapeutics application user research study.
10. Has participated in another clinical study (interventional or observational) in the last 6 months (26 weeks).
11. Currently needs or will likely require prohibited concomitant medications and/or therapy during the trial, as determined by the investigator.
12. Has suicidal ideation or behavior, as assessed by the C-SSRS:
13. Participants with a “yes” response to either Items 4 or 5 on the C-SSRS Suicidal Ideation Item within the last 3 months (12 weeks) prior to screening, or at the Baseline Visit.
14. Participants with a “yes” response on the C-SSRS Suicidal Behavior Items within the last 6 months (26 weeks) prior to screening or at the Baseline Visit.
15. Participants who, in the opinion of the investigator, present a serious risk of suicide.

### Lifestyle Considerations

Participants should have routine access to their smartphones for the duration of the trial. In addition, they should be able to attend in-clinic and remote telemedicine visits during the trial. Participants should refrain from using alcohol and recreational drugs during the times the Study App will be accessed. Occasional recreational use of THC is permitted.

### Study Intervention(s) and Concomitant Therapy

### Study Intervention(s) Administered

The digital therapeutics application-R-001 Study Mobile Application (The Study App) will administer participants with one of two study interventions. Study interventions are digital therapeutics application PDT and a comparator Digital Control. (Table 2).

TABLE 2
Study Interventions

| | Digital Therapeutics | |
|---|---|---|
| | Application | Digital Control |
| Type | SaMD | DTx |
| Dose Formulation | PDT | Control |
| Unit Dose Strength(s) | N/A | N/A |
| Dosage Level(s) | N/A | N/A |
| Route of Administration | Digital | Digital |
| Use | Experimental | Comparator |
| IMP and NIMP | N/A | N/A |
| Packaging and Labeling | Digital | Digital |
| Current/Former Name(s) or | N/A | N/A |
| Alias(es) | | |

DTx = digital therapeutic;
IMP = investigational medicinal product;
N/A = not applicable;
NIMP = non-investigational medicinal product;
PDT = prescription digital therapeutic;
SaMD = software-as-a-medical device

The digital therapeutics application is being developed as an adjunctive prescription digital therapeutic designed to target experiential negative symptoms in adult and late adolescent patients with schizophrenia who are stable on SOC. Therapeutic delivery is guided by a validated mechanistic model to address important clinical needs identified by people with schizophrenia. Therapeutic techniques in CT-155 were selected based on clinical evidence, and the individual components were designed based on the underlying principles of face-to-face treatment to provide maximal support to people with schizophrenia.

The digital therapeutics application will achieve the primary objective of improving experiential negative symptoms by integrating multiple neurobehavioral therapeutic techniques with established evidence, as described in further detail below. These therapeutic techniques will work together to help people set goals (adaptive goal setting) that promote real-world engagement (behavioral activation) while removing barriers (cognitive restructuring) and providing skills that will facilitate goal attainment (social skills training, positive affect training).

### Digital Control

A Digital Control application will be used as a comparator group in this trial. The application will control for common elements of a digital application (receipt of notifications, on-demand access), and will control for daily engagement with a novel application.

### Preparation/Handling/Accountability/Disposition

### Generally:

The designated unblinded personnel must confirm download and activation of the Study App.
Only participants enrolled in the study may receive study intervention.
The Study App will automatically become inert after the completion of Week 20. Designated unblinded trial site staff will instruct participants to uninstall the Study App during their final study visit, or upon discontinuation.

### Measures to Minimize Bias: Randomization and Blinding

Study using IVRS/IWRS: All participants will be centrally assigned to randomized study intervention using an Interactive Voice/Web Response System (IVRS/IWRS). Before the study is initiated, the telephone number and call-in directions for the IVRS and/or the log in information and directions for the IWRS will be provided to each site.

Study intervention (Study App) will be downloaded, activated, and deleted at the study visits summarized in the SoA.

Blind Break (IVRS/IWRS) The IVRS/IWRS will be programmed with blind-breaking instructions. In case of an emergency, the investigator has the sole responsibility for determining if unblinding of a participants' intervention assignment is warranted. Participant safety must always be the first consideration in making such a determination. If the investigator decides that unblinding is warranted, the investigator should make every effort to contact the sponsor prior to unblinding a participant's intervention assignment unless this could delay emergency treatment of the participant. Once a participant's intervention assignment is unblinded, the sponsor must be notified within 24 hours after breaking the blind. The date and reason that the blind was broken must be recorded in the source documentation and case report form, as applicable.

Blinded study with unblinded study personnel's role. Participants will be randomly assigned in a 1:1 ratio to receive either the digital therapeutics application or Digital Control App as study intervention through the Study App. Investigators, designated site personnel, and raters will remain blinded to each participant's assigned study intervention throughout the course of the study. To maintain this blind, designated unblinded site personnel will assist the study participants with downloading and verifying installation of the Study App. Designated unblinded site personnel will be responsible for conducting adherence checks.

Describe method for blinded assessments: To further protect from bias, a centralized blinded rater team will administer the CAINS assessments.

Study Hypothesis Blind: Study participants will be blinded to the efficacy hypothesis of the study. Both treatment arms will be presented to the participant as possible treatments for schizophrenia. No references to digital therapeutics application or Digital Control will be made to the participant. This approach limits the risk of participant unblinding to treatment assignment and expected efficacy.

Assigned safety personnel may unblind the intervention assignment for any participant with an SAE. If the SAE requires that an expedited regulatory report be sent to one or more regulatory agencies, a copy of the report, identifying the participant's intervention assignment, may be sent to investigators in accordance with local regulations and/or sponsor policy.

### Study Intervention Adherence

During the treatment period, all randomized participants will be instructed to use the Study App as instructed by the Study App. Participants in both the digital therapeutics application and Digital Control arms will be considered adherent for a given day of treatment if they complete at least 1 available daily activity. A participant will be considered adherent for the study if they are adherent for at least 67 of the 112 total treatment days (˜60%). Assigned activity completion is measured and recorded using in-app engagement metrics and is defined in the statistical analysis plan (SAP).

### Adherence Monitoring

In order to check if participants are adherent with treatment as assigned by the Study App, site staff will conduct an adherence check at weeks 4, 8 and 12. During these visits, designated unblinded site staff will obtain adherence data from the participant via an adherence code read from the Study App. This code will indicate how adherent the participant has been with treatment up to the point the code is accessed. Investigators will use this information to remind the participants that they should be using the Study App every day and identify any technical problems they may be having interfering with adherence to treatment. Guidance and language for this conversation will be specified in the Study App Investigator Guide.

### Concomitant Therapy

Participants must be on a stable dose of antipsychotic medication(s) for at least 12 weeks prior to randomization (Day 1). Dose adjustments will be permitted during the study, as outlined within the respective package inserts of their current medication(s).

Participants are not allowed to be treated with more than two (2) antipsychotic medications (including more than two dosage forms). Treatment with clozapine or haloperidol is prohibited.

Any additional psychotherapy is not allowed during the study, including Cognitive Behavioral Therapy, Social Skills Training, and Motivational Enhancement Therapy.

Occasional use of recreational drugs other than synthetic cathinones (bath salts), synthetic cannabinoids (K2, Spice), inhalants, amphetamines (including MDMA/ecstasy), phencyclidine (PCP), cocaine, opiates, benzodiazepines, barbiturates, hallucinogens, or parenteral drugs will not be grounds for removal from the trial.

Participant Discontinuation/Withdrawal from the Study

Every effort should be made by the site staff to encourage participants to remain in the study and on study intervention if medically safe. Participants who prematurely discontinue study intervention must complete the early termination procedures as described in the SoA. Participants who discontinue study intervention prematurely should ideally be observed until the end of the trial as if they were still receiving blinded study treatment. For all participants, the reason for withdrawal from study intervention (e.g., AEs) must be recorded in the CRF. These data will be included in the trial database and reported.

Participants who are not actively using study intervention may be less motivated to adhere to the study visit schedule. Investigators and site staff should work to detect early signs of waning interest and readily present such participants with the following options to encourage continued participation:
Early D/C Option 1: Continue to conduct regularly scheduled visits
Early D/C Option 2: Conduct all remaining study visits. At the time of planned clinic visits, only the following assessments need to be conducted:
   PANSS
   CAINS and CAINS-MAP
   CGI-S
   C-SSRS
   Adverse Events
   Concomitant Therapy
Early D/C Option 3: Discontinue participation in remaining study activities, but permit collection of the occurrence of psychiatric illness/relapse and vital status approximately 16 weeks after randomization through the participant or alternative person designated by the participant (e.g., family, spouse, partner, legal representative or physical).
Early D/C Option 4: Same as option 3 above, but with the possibility of collection of the occurrence of psychiatric illness/relapse and vital status approximately 16 weeks after randomization through review of participant's medical information from alternative sources (e.g., doctors notes, hospital records, etc.).

Participants will be asked to choose the most rigorous form of follow-up that they are willing to comply with. Participants who refuse all four of the above are considered to have fully withdrawn consent to participate in the study.

The Study App on the discontinued participant's smartphone device will be disabled.

### Study Assessments and Procedures

Study assessments and procedures, including their timing, are summarized in the SoA. Adherence to the study design requirements, including those specified in the SoA, is essential and required for study conduct. Protocol waivers or exemptions are not allowed. Every effort should be made to ensure that the protocol required assessments and procedures are completed as described. All the clinician-administered scales should be administered by individuals who have been appropriately trained.

### Efficacy Assessments

The following efficacy assessment scales are used in this trial at the times provided in the SoA. A description of the scales and the respective scoring algorithms for all endpoints will be provided in the Statistical Analysis Plan (SAP).

### Clinical Assessment Interview for Negative Symptoms (CAINS)

The CAINS is a 13-item interview-based assessment comprised of two subscales that measure the two factors of negative symptoms: Motivation and Pleasure (MAP) and Expressivity (EXP). CAINS items are scored on a 5-point scale, with lower scores indicating lower negative symptom severity. Note that the CAINS is administered by a centralized blinded rater team, not the site clinician.

The MAP scale consists of nine items that measure interest and engagement in motivated behavior as well as the experience of pleasure across social, vocational, and recreational domains. Each item is scored based on patient-reported behavior and experience. The EXP scale consists of four items that measure verbal intonation (prosody), non-verbal expressivity (gestures, posture), facial expressivity, and speech output (alogia). Items are rated based on observation over the course of the interview.

### Positive and Negative Syndrome Scale (PANSS)

The PANSS consists of three subscales containing a total of 30 symptom constructs. For each symptom construct, severity is rated on a 7-point scale, with a score of one indicating the absence of symptoms and a score of seven indicating extremely severe symptoms. The symptom constructs for each subscale is as follows:

Positive Subscale (7 positive symptom constructs: delusions, conceptual disorganization, hallucinatory behavior, excitement, grandiosity, suspiciousness/persecution, and hostility).

Negative Subscale (7 negative symptom constructs: blunted affect, emotional withdrawal, poor rapport, passive/apathetic social withdrawal, difficulty in abstract thinking, lack of spontaneity and flow of conversation, stereotyped thinking).

General Psychopathology Subscale (16 symptom constructs: somatic concern, anxiety, guilt feelings, tension, mannerisms and posturing, depression, motor retardation, uncooperativeness, unusual thought content, disorientation, poor attention, lack of judgment and insight, disturbance of volition, poor impulse control, preoccupation, and active social avoidance).

### Personal and Social Performance Scale (PSP)

The PSP is a validated clinician-rated scale that measures personal and social functioning in four domains: socially useful activities (e.g., work and study), personal and social relationships, self-care, and disturbing and aggressive behaviors. Each area is scored on a 0-100 scale, with anchors for every 10-point interval.

### Defeatist Beliefs Subscale of the Dysfunctional Attitudes Scale (DAS)

The Defeatist Performance Beliefs subscale is a 15-item subset of the Dysfunctional Attitudes Scale. Items are scored on a 1-7 scale, with higher scores indicating more severe defeatist thinking.

### Mini-International Neuropsychiatric Interview (MINI)

The MINI (Version 7.0.2 for DSM-5) will be used as a tool for conducting a structured interview for eligibility assessment at the screening visit. The MINI is a widely used structured diagnostic interview instrument developed for DSM-5 psychiatric disorders. A qualified rater will conduct the interview.

The results of the MINI will be compared with the inclusion/exclusion criteria for evaluation of comorbid diagnoses.

### Clinical Global Impression of Severity (CGI-S)

The CGI-S is a standardized, clinician-rated global rating scale that measures experiential negative symptom severity in the past seven days using a 7-point Likert scale. A higher score on the CGI-S represents a higher severity of disease. Response choices include: 0=not assessed; 1=normal, not at all ill; 2=borderline mentally ill; 3=mildly ill; 4=moderately ill; 5=markedly ill; 6=severely ill; and 7=among the most extremely ill participants.

### Patient Global Impression of Improvement (PGI-I)

The PGI-I is a patient-reported outcome measuring participative subjective improvement in experiential negative symptoms severity on a 7-point scale. Higher scores on the PGI-I indicate a subjective report of disease worsening over the course of treatment.

### Patient Global Impression of Severity (PGI-S)

The PGI-S is a patient-reported outcome measuring participative subjective severity of experiential negative symptoms on a 5-point scale. Higher scores on the PGI-S indicate a subjective report of higher severity of disease.

### EQ-5D-5L

The EQ-5D-5L is a standardized, brief self-report instrument for measuring health status. It consists of two components: the EQ-5D descriptive system and the EQ visual analogue scale. The descriptive system comprises five dimensions: mobility, self-care, usual activities, pain/discomfort, and anxiety/depression. The participant is asked to indicate his/her health status by selecting the box next to the most appropriate statement in each of the five dimensions. The EQ visual analogue scale records the participant's overall self-rated health on a visual analogue scale, from “the best health you can imagine” to “the worst health you can imagine.”

### Sheehan Disability Scale (SDS)

The SDS is a brief, 5-item self-report instrument that assesses functional impairment in three domains: work/school, social life, and family life.

### WHO Disability Assessment Schedule (WHODAS 2.0)

The WHODAS 2.0 is a 36-item self-assessment scale to measure a participant's function and disability across 6 domains of life: cognition (understanding and communicating), mobility (moving and getting around), self-care (hygiene, dressing, eating, staying alone), getting along (interacting with others), life activities (domestic responsibilities, leisure, work and school), and participation (community and society).

### Schizophrenia Quality of Life Scale Revision 4 (SQLS-R4)

The SQLS-R4 is a 33-item self-assessment scale that assesses quality of life across domains of psychosocial feelings and vitality/cognition domains using a 5-point scale.

### Brief Assessment of Cognition in Schizophrenia (BACS) Subscales

The Brief Assessment of Cognition in Schizophrenia (BACS) is a validated pen/paper cognitive assessment, containing several subtests, including symbol coding task assessing working memory; the verbal memory task; and the digit sequencing task, assessing attention and processing speed of information. These three subtests can be combined into a short-form global screener of cognitive functioning (BACS-SF).

Symbol coding task: Participants write numerals 1-9 matching to symbols on a response sheet for 90 seconds. Total task duration is 3 minutes.

Verbal memory task: Participants are presented with 15 words and then asked to recall as many as possible. Procedure is repeated 5 times. Total task duration is 7 minutes.

Digit Sequencing: Participants are presented with clusters of numbers of increasing length and are required to tell the experimenter the numbers in order, from lowest to highest. Total task duration is 5 minutes.

### Study App Engagement Assessments

Participant engagement with the Study App will be captured automatically by the Study App. The metrics below will be captured.
Number of times the app is opened by the user
Number of days the app is opened by the user
Number of lessons completed per user
Number of times a skill is practiced per user
Number of modules completed
Number of times the mood check-in is completed during the daily check-in per user
Number of adaptive goal setting (AGS) daily activities completed
Number of goals completed per user

### Statistical Considerations

Participant health economic data will be assessed as exploratory endpoints.

### Statistical Hypotheses

The null hypothesis is that there is no difference between the means of the treatment arms and the alternative is that the means are different.

The null hypothesis will be rejected in favor of the alternative hypothesis if the two-sided p-value is smaller than 0.05 and the result favors the digital therapeutics application arm.

### Sample Size Determination

The primary endpoint is change from baseline to Week 16 in experiential negative symptoms, as assessed by CAINS-MAP scale. The null hypothesis is that there is no difference between the means of the treatment arms and the alternative is that the means are different. To demonstrate this, and assuming a standardized effect (Cohen's d) between the treatment arms of 0.35, 173 participants per arm are required to achieve 90% power when using a type I error rate of 5% (two-sided). Assuming 20% early termination, 432 participants will need to be randomized to the study in a 1:1 ratio (approximately 216 per arm). Sample size was calculated using t-test for two independent samples and assuming equal variance.

### Analysis Sets

For the purposes of analysis, the following analysis sets are defined:

### Analysis Set: Description

Enrolled: All participants who signed the ICF.

Intent-To-Treat (ITT) All randomized participants, based on the assigned intervention in the randomization and recorded in the database, regardless of successful activation or use of the Study App. Participants treated without being randomized will not be considered as randomized and will not be included in any efficacy or safety analyses.

This analysis set will be used as a supportive analysis for the primary endpoint.

Modified Intent-to-Treat (mITT): All randomized participants, based on the assigned intervention in the randomization and recorded in the database, for whom the app was activated by successful entry of the access code in the study application and have used the application at least once, with at least one evaluable baseline measurement. This will be the main analysis set for all efficacy analysis.

Per Protocol (PP): All randomized participants who completed the 16-week treatment with an adherence level of at least 60% during the 16 weeks of treatment and who did not have a major protocol deviation which impact the calculation of the primary endpoint. This analysis set will be used as a supportive analysis for the primary endpoint.
Participants with the following deviations will be excluded from the PP set:
Participants who received intervention different from the randomized intervention
Participants who do not meet the inclusion/exclusion criteria
Participants who used disallowed medication.
Additional criteria for exclusion from the PP set may be defined in the SAP.

### General Considerations

For continuous variables, summary tables will provide the number of observations [n], mean, standard deviation, median, minimum and maximum. For categorical variables, summary tables will provide the number of observations [n] and frequency of each category (including missing data). Tabulations will be presented by treatment and overall, when appropriate. Modeling and testing will be described for each endpoint.

### Participant's Disposition

The number of participants who were screened and enrolled to the study will be presented, as well as the reason for not being randomized. The number of participants who discontinue the study will be presented by reason for discontinuation. The number of participants in the ITT, mITT, PP, and safety analysis set will be presented.

### Demographics and Baseline Characteristics

In order to assess the comparability of the two arms at baseline, demographic and baseline characteristics, data will be summarized by treatment group. These summaries will be presented for the mITT set. If there is a difference of more than five participants in the total number of participants between the mITT and the safety, ITT and PP analysis sets, the summaries will be presented for these sets.

### Primary Endpoint(s)

The primary analysis will be conducted on the mITT set. The mITT set will be supported by an analysis on the ITT set to allow for an evaluation of similarity.

The primary endpoint is change from baseline to Week 16 in experiential negative symptoms, as assessed by CAINS-MAP scale. The null hypothesis is that there is no difference between the means of the treatment arms and the alternative is that the means are different. To demonstrate this, and assuming a standardized effect (Cohen's d) between the treatment arms of 0.35, 173 participants per arm are required to achieve 90% power when using a type I error rate of 5% (two-sided). Assuming 20% early termination, 432 participants will need to be randomized to the study in a 1:1 ratio (approximately 216 per arm). Sample size was calculated using t-test for two independent samples and assuming equal variance.

The primary endpoint of change from baseline to Week 16 in experiential negative symptoms will be analyzed using Mixed Models Repeated Measures (MMRM) with response variable of change from baseline at each visit in which the CAINS-MAP is evaluated. The model will include an intercept and the following covariates: experiential negative symptom at baseline, visit (as class variable), treatment arm, treatment by visit interaction terms, and baseline by visit interaction terms.

The first choice for the within-patient correlations will be an unstructured covariance matrix. Mitigation in the case of no convergence will be discussed in the SAP. Only data at scheduled visits will be used for this analysis. The study will be considered successful if the two-sided p-value for the difference in the change from baseline at Week 16 between the two treatment arms is smaller than 0.05, and a larger decrease in CAINS-MAP is observed in the digital therapeutics application arm than the Digital Control arm.

Missing data will not be imputed for the primary analysis. All measurements available for each participant will be used.

The planned number of sites is approximately 40. The SAP will consider pooling of sites (e.g., by region or type of center) and assess the impact of pooled sites in an exploratory manner. The analysis will be repeated on the PP set.

### Sensitivity Analysis

Missing data will be handled via multiple imputations (MI) under Missing At Random (MAR) where imputations will be carried out within the randomized arm, and the primary model will be repeated for each complete data set. Details will be provided in the Statistical Analysis Plan.

In order to assess the robustness of the results to missing data under the MAR assumption, a tipping point analysis will be implemented. In this analysis, all missing data in the sham group will be imputed based on observations in that group (representing MAR assumption). Missing data in the digital therapeutics application group will be imputed under a different set of assumptions, starting from MAR assumption and gradually reducing the effect until the tipping point is reached. Details will be provided in the SAP.

### Subgroups

The primary analysis will be repeated by the following subgroups:
Length of time since diagnosis of schizophrenia (0-5 years, 5+ years)
Baseline CAINS-MAP negative symptom severity (moderate, moderate-severe, severe)
Severity of cognitive impairment (to be defined in the SAP)
Sex (Male/Female)
Age group (18-21 years, 22-64 years, 65+ years)

### Secondary Endpoints

Analysis of secondary endpoints will be conducted using the mITT analysis set. The secondary endpoints will be analyzed in a similar manner to the primary endpoint. The analysis will be difference between the treatment arms using the MMRM. Additional details will be provided in the SAP.

### Multiplicity Control

No multiplicity adjustment will be made to secondary endpoints, sensitivity analyses, and subgroups.

It is anticipated that users presented with the digital therapeutic application (e.g., the application 165) in accordance with the methods laid out herein, will show amelioration of experiential negative symptoms of schizophrenia. The user may be taking antipsychotic medication (e.g., isperidone (Risperdal), quetiapine (Seroquel), olanzapine (Zyprexa), ziprasidone (Zeldox), paliperidone (Invega), or aripiprazole (Abilify)) in conjunction with the digital therapeutic application. The amelioration will be measured using any number of metrics, such as Clinical Assessment Interview for Negative Symptoms, Motivation and Pleasure Scale (CAINS-MAP), Defeatist Beliefs Subscale of the Dysfunctional Attitudes Scale (DAS), relative to a comparator digital application; or assessing Patient Global Impression of Improvement Scale (PGI-I), among others. The metrics may be measured after one or more activities performed, as prompted by the digital therapeutic application.

Example embodiments may include a method for ameliorating experiential negative symptoms of schizophrenia in a subject receiving antipsychotic medication comprising exposing the subject to a prescription digital therapeutic (DTx) disclosed herein. The antipsychotic medication is isperidone (Risperdal), quetiapine (Seroquel), olanzapine (Zyprexa), ziprasidone (Zeldox), paliperidone (Invega), and aripiprazole (Abilify), among others. In some embodiments, the method may include assessing the severity of experiential negative symptoms of schizophrenia in the subject following exposure to the prescription DTx. In some embodiments, the severity of experiential negative symptoms of schizophrenia may be determined using Clinical Assessment Interview for Negative Symptoms, Motivation and Pleasure Scale (CAINS-MAP).

In some embodiments, the method may include assessing a change from baseline in motivation and pleasure symptoms at Week 8, as assessed by CAINS-MAP, relative to a comparator digital application; or assessing a change from baseline in expressive negative symptoms at Weeks 8 and 16, as assessed by the Clinical Assessment Interview for Negative Symptoms, Expressivity Scale (CAINS-EXP), relative to a comparator digital application; or assessing a change from baseline in positive symptoms at Weeks 8 and 16, as assessed by the Positive and Negative Syndrome Scale (PANSS), relative to a comparator digital application; or assessing a change from baseline in social functioning at Weeks 8 and 16, as assessed by the Personal and Social Performance Scale (PSP), relative to a comparator digital application; or assessing a change from baseline in self-reported defeatist beliefs at Weeks 8 and 16, as assessed by the Defeatist Beliefs Subscale of the Dysfunctional Attitudes Scale (DAS), relative to a comparator digital application; or assessing Patient Global Impression of Improvement Scale (PGI-I) at Weeks 8 and 16, relative to a comparator digital application.

In some embodiments, the experiential negative symptoms of schizophrenia may include blunted affect, alogia (reduction in quantity of words spoken), avolition (reduced goal-directed activity due to decreased motivation), asociality, and anhedonia (reduced experience of pleasure). In some embodiments, the subject may have been receiving a stable dose of antipsychotic medication(s) for at least 12 weeks prior to exposure to the prescription DTx.

Example embodiments may include a method for ameliorating experiential negative symptoms of schizophrenia in a subject in need, thereof comprising exposing the subject to a prescription digital therapeutic (DTx) disclosed herein. In some embodiments, the method may include assessing the severity of experiential negative symptoms of schizophrenia in the subject following exposure to the prescription DTx. In some embodiments, the severity of experiential negative symptoms of schizophrenia may be determined using Motivation and Pleasure Scale-Self Report (MAP-SR). In some embodiments, the severity of experiential negative symptoms of schizophrenia may be determined using Clinical Assessment Interview for Negative Symptoms (CAINS) assessment. In some embodiment, the experiential negative symptoms of schizophrenia may include blunted affect, alogia (reduction in quantity of words spoken), avolition (reduced goal-directed activity due to decreased motivation), asociality, and anhedonia (reduced experience of pleasure).

Example embodiments may include a method for ameliorating experiential negative symptoms of schizophrenia in a subject receiving antipsychotic medication comprising exposing the subject to a prescription digital therapeutic (DTx) disclosed herein. In some embodiments, the antipsychotic medication may be isperidone (Risperdal), quetiapine (Seroquel), olanzapine (Zyprexa), ziprasidone (Zeldox), paliperidone (Invega), aripiprazole (Abilify), or iclepertin, among others. The method may include assessing the severity of experiential negative symptoms of schizophrenia in the subject following exposure to the prescription DTx. In some embodiments, the severity of experiential negative symptoms of schizophrenia are determined using Clinical Assessment Interview for Negative Symptoms, Motivation and Pleasure Scale (CAINS-MAP).

In some embodiments, the method may include assessing a change from baseline in motivation and pleasure symptoms at Week 8, as assessed by CAINS-MAP, relative to a comparator digital application; or assessing a change from baseline in expressive negative symptoms at Weeks 8 and 16, as assessed by the Clinical Assessment Interview for Negative Symptoms, Expressivity Scale (CAINS-EXP), relative to a comparator digital application; or assessing a change from baseline in positive symptoms at Weeks 8 and 16, as assessed by the Positive and Negative Syndrome Scale (PANSS), relative to a comparator digital application; or assessing a change from baseline in social functioning at Weeks 8 and 16, as assessed by the Personal and Social Performance Scale (PSP), relative to a comparator digital application; or assessing a change from baseline in self-reported defeatist beliefs at Weeks 8 and 16, as assessed by the Defeatist Beliefs Subscale of the Dysfunctional Attitudes Scale (DAS), relative to a comparator digital application; or assessing Patient Global Impression of Improvement Scale (PGI-I) at Weeks 8 and 16, relative to a comparator digital application.

In some embodiments, the experiential negative symptoms of schizophrenia may include blunted affect, alogia (reduction in quantity of words spoken), avolition (reduced goal-directed activity due to decreased motivation), asociality, and anhedonia (reduced experience of pleasure). In some embodiments, the subject has been receiving a stable dose of antipsychotic medication(s) for at least 12 weeks prior to exposure to the prescription DTx.

### C. Network and Computing Environment

Various operations described herein can be implemented on computer systems. FIG. 24 shows a simplified block diagram of a representative server system 2400, client computing system 2414, and network 2426 usable to implement certain embodiments of the present disclosure. In various embodiments, server system 2400 or similar systems can implement services or servers described herein or portions thereof. Client computing system 2414 or similar systems can implement clients described herein. The system 100 described herein can be similar to the server system 2400. Server system 2400 can have a modular design that incorporates a number of modules 2402 (e.g., blades in a blade server embodiment); while two modules 2402 are shown, any number can be provided. Each module 2402 can include processing unit(s) 2404 and local storage 2406.

Processing unit(s) 2404 can include a single processor, which can have one or more cores, or multiple processors. In some embodiments, processing unit(s) 2404 can include a general-purpose primary processor as well as one or more special-purpose co-processors such as graphics processors, digital signal processors, or the like. In some embodiments, some or all processing units 2404 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 2404 can execute instructions stored in local storage 2406. Any type of processors in any combination can be included in processing unit(s) 2404.

Local storage 2406 can include volatile storage media (e.g., DRAM, SRAM, SDRAM, or the like) and/or non-volatile storage media (e.g., magnetic or optical disk, flash memory, or the like). Storage media incorporated in local storage 2406 can be fixed, removable, or upgradeable as desired. Local storage 2406 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 2404 need at runtime. The ROM can store static data and instructions that are needed by processing unit(s) 2404. The permanent storage device can be a non-volatile read-and-write memory device that can store instructions and data even when module 2402 is powered down. The term “storage medium” as used herein, includes any medium in which data can be stored indefinitely (subject to overwriting, electrical disturbance, power loss, or the like) and does not include carrier waves and transitory electronic signals propagating wirelessly or over wired connections.

In some embodiments, local storage 2406 can store one or more software programs to be executed by processing unit(s) 2404, such as an operating system and/or programs implementing various server functions, such as functions of the system 100 or any other system described herein, or any other server(s) associated with system 100 or any other system described herein.

“Software” refers generally to sequences of instructions that, when executed by processing unit(s) 2404, cause server system 2400 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 2404. Software can be implemented as a single program, or a collection of separate programs or program modules that interact as desired. From local storage 2406 (or non-local storage described below), processing unit(s) 2404 can retrieve program instructions to execute and data to process in order to execute various operations described above.

In some server systems 2400, multiple modules 2402 can be interconnected via a bus or other interconnect 2408, forming a local area network that supports communication between modules 2402 and other components of server system 2400. Interconnect 2408 can be implemented using various technologies, including server racks, hubs, routers, etc.

A wide-area network (WAN) interface 2410 can provide data communication capability between the local area network (e.g., through the interconnect 2408) and the network 2426, such as the Internet. Other technologies can be used to communicatively couple the server system 2400 with the network 2426, including wired (e.g., Ethernet, IEEE 802.3 standards) and/or wireless technologies (e.g., Wi-Fi, IEEE 802.11 standards).

In some embodiments, local storage 2406 is intended to provide working memory for processing unit(s) 2404, providing fast access to programs and/or data to be processed, while reducing traffic on interconnect 2408. Storage for larger quantities of data can be provided on the local area network by one or more mass storage subsystems 2412 that can be connected to interconnect 2408. Mass storage subsystem 2412 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 2412. In some embodiments, additional data storage resources may be accessible via WAN interface 2410 (potentially with increased latency).

Server system 2400 can operate in response to requests received via WAN interface 2410. For example, one of modules 2402 can implement a supervisory function and assign discrete tasks to other modules 2402 in response to received requests. Work allocation techniques can be used. As requests are processed, results can be returned to the requester via WAN interface 2410. Such operation can generally be automated. Further, in some embodiments, WAN interface 2410 can connect multiple server systems 2400 to each other, providing scalable systems capable of managing high volumes of activity. Other techniques for managing server systems and server farms (collections of server systems that cooperate) can be used, including dynamic resource allocation and reallocation.

Server system 2400 can interact with various user-owned or user-operated devices via a wide-area network, such as the Internet. An example of a user-operated device is shown in FIG. 24 as client computing system 2414. Client computing system 2414 can be implemented, for example, as a consumer device such as a smartphone, other mobile phone, tablet computer, wearable computing device (e.g., smart watch, eyeglasses), desktop computer, laptop computer, and so on.

For example, client computing system 2414 can communicate via WAN interface 2410. Client computing system 2414 can include computer components such as processing unit(s) 2416, storage device 2418, network interface 2420, user input device 2422, and user output device 2424. Client computing system 2414 can be a computing device implemented in a variety of form factors, such as a desktop computer, laptop computer, tablet computer, smartphone, other mobile computing device, wearable computing device, or the like.

Processing unit(s) 2416 and storage device 2418 can be similar to processing unit(s) 2404 and local storage 2406 described above. Suitable devices can be selected based on the demands to be placed on client computing systems; for example, client computing system 2414 can be implemented as a “thin” client with limited processing capability or as a high-powered computing device. Client computing system 2414 can be provisioned with program code executable by processing unit(s) 2416 to enable various interactions with server system 2400.

Network interface 2420 can provide a connection to the network 2426, such as a wide-area network (e.g., the Internet) to which WAN interface 2410 of server system 2400 is also connected. In various embodiments, network interface 2420 can include a wired interface (e.g., Ethernet) and/or a wireless interface implementing various RF data communication standards such as Wi-Fi, Bluetooth, or cellular data network standards (e.g., 3G, 4G, 5G, 24G, LTE, etc.).

User input device 2422 can include any device (or devices) via which a user can provide signals to client computing system 2414; client computing system 2414 can interpret the signals as indicative of particular user requests or information. In various embodiments, user input device 2422 can include any or all of a keyboard, touch pad, touch screen, mouse or other pointing device, scroll wheel, click wheel, dial, button, switch, keypad, microphone, and so on.

User output device 2424 can include any device via which client computing system 2414 can provide information to a user. For example, user output device 2424 can include display-to-display images generated by, or delivered to, client computing system 2414. The display can incorporate various image generation technologies, e.g., a liquid crystal display (LCD), light-emitting diode (LED), including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 2424 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 2404 and 2416 can provide various functionality for server system 2400 and client computing system 2414, including any of the functionality described herein as being performed by a server or client, or other functionality.

It will be appreciated that server system 2400 and client computing system 2414 are illustrative and that variations and modifications are possible. Computer systems used in connection with embodiments of the present disclosure can have other capabilities not specifically described here. Further, while server system 2400 and client computing system 2414 are described with reference to particular blocks, it is to be understood that these blocks are defined for convenience of description and are not intended to imply a particular physical arrangement of component parts. For instance, different blocks can be, but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus, including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including, but not limited to, specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media, such as compact disk (CD) or digital versatile disk (DVD), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

1. A method for ameliorating experiential negative symptoms of schizophrenia in a user in need thereof, comprising:
obtaining, by one or more processors, a first metric associated with the user prior to a plurality of time instances;
determining at least one endpoint associated with the user;
identifying a configuration file selected from a plurality of configuration files based on the at least one endpoint, the configuration file identifying a set of content items for an activity of a plurality of activities towards achieving the at least one endpoint and corresponding logic for presenting at least one content item from the set of content items, the corresponding logic comprising a plurality of stages corresponding with the at least one endpoint;
generating, for execution on an application, at least one instruction for presenting at least one content item from the set of content items based on the corresponding logic;
presenting, via the application responsive to executing the at least one instruction, the at least one content item identified by the configuration file to prompt the user to perform the activity towards achieving the at least one endpoint, wherein the at least one content item corresponds to a first stage of the plurality of stages;
repeating, by the one or more processors, for each time instance of the plurality of time instances:
receiving response data identifying one or more interactions by the user with the set of content items; and
based on the response data, presenting at least one content item corresponding to the first stage or a subsequent stage of the plurality of stages; and
obtaining, by the one or more processors, a second metric associated with the user after at least one of the plurality of time instances;
wherein the user shows amelioration of experiential negative symptoms of schizophrenia, when the second metric is statistically different from the first metric.

2. The method of claim 1, wherein the first metric and second metric comprise scores on at least one of or a portion of at least one of a Clinical Assessment Interview for Negative Symptoms (CAINS) Motivation and Pleasure Scale, Clinical Assessment Interview for Negative Symptoms, Expressivity Scale (CAINS-EXP), Positive and Negative Syndrome Scale (PANSS), Personal and Social Performance Scale (PSP), a Defeatist Beliefs Subscale of the Dysfunctional Attitudes Scale (DAS), Patient Global Impression of Improvement Scale (PGI-I), Patient Global Impression of Severity Scale (PGI-S), Clinical Global Impression of Severity Scale (CGI-S), WHO Disability Assessment Schedule 2.0 (WHODAS 2.0), [or] Schizophrenia Quality of Life Scale-Revision 4 (SQLS-R4).

3. The method of claim 1, wherein the experiential negative symptoms of schizophrenia comprise one or more of blunted affect, alogia, avolition, asociality, and anhedonia.

4. The method of claim 1, wherein the user is an adult or a late adolescent.

5. The method of claim 1, wherein the user has experienced at least moderate to severe negative symptom severity prior to the first activity.

6. The method of claim 1, wherein the user has a score of less than or equal to 30 on a Motivation and Pleasure Scale (MAPS) prior to the activity.

7. The method of claim 1, wherein the user has been receiving a stable dose of an antipsychotic medication for at least 12 weeks prior to the activity.

8. The method of claim 7, wherein the antipsychotic medication comprises risperidone, quetiapine, olanzapine, ziprasidone, paliperidone, aripiprazole, or iclepertin.

9. The method of claim 1, wherein the user is male, female, or non-binary.

10. The method of claim 1, wherein at least one of the plurality of configuration files identifies a criterion defining a measure to select another of the plurality of configuration files, the measure identifying at least one of (i) a likelihood that the user is to perform the activity or (ii) a predicted efficacy of the activity on the user towards achieving the at least one endpoint.

11. The method of claim 1, wherein the at least one endpoint for a first time instance of the plurality of time instances is based on a baseline assessment of the user for the experiential negative symptoms of schizophrenia.

12. The method of claim 1, wherein the at least one endpoint is based on a personal value indicated by the user.

13. The method of claim 1, wherein the at least one endpoint is identified or updated responsive to a transition from the first stage to the subsequent stage, wherein the transition is determined based on the response data.

14. The method of claim 1, wherein the at least one endpoint is based on a first mood indicated by the user.

15. The method of claim 1, wherein the at least one endpoint is associated with at least one of a plurality of domains, the plurality of domains comprising a social domain, a recreation domain, and a productivity domain.

16. The method of claim 1, wherein the user is presented with a comparison of a first rating performed prior to performance of a respective second activity and a second rating performed after performance of the respective second activity.

17. The method of claim 1, further comprising determining, by the one or more processors, to continue the repeating through the plurality of time instances based on an amount of time from the obtaining of the first metric, and wherein repeating for each time instance of the plurality of time instances further comprises repeating the time instance responsive to determination to continue.

18. The method of claim 1, wherein repeating for each time instance of the plurality of time instances further comprises updating, for a time instance, the at least one endpoint based on the response data identifying the one or more interactions by the user with the set of content items presented via an application from a previous time instance.

19. The method of claim 1, wherein repeating for each time instance of the plurality of time instances further comprises converting human-readable instructions of the configuration file to generate a package including machine executable format instructions.

20. The method of claim 1, wherein obtaining the first metric and the second metric further comprises obtaining the first metric and the second metric from a source separate from the application.
